(19) United States
(12) Patent Application Publication
(10) Pub. No.: US 2026/0273054 A1
(43) Pub. Date: Sep. 17, 2026

(54) IDENTIFICATION OF NEOANTIGEN-REACTIVE T CELL RECEPTORS

(71) Applicant: Alaunos Therapeutics, Inc., Fort Lauderdale, FL (US)

(72) Inventors: Matthew R. COLLINSON-PAUTZ, Fort Lauderdale, FL (US); Drew C. DENIGER, Fort Lauderdale, FL (US); Guowei GU, Fort Lauderdale, FL (US); Donghyun JOO, Fort Lauderdale, FL (US); Charles HO, Fort Lauderdale, FL (US)

(73) Assignee: Alaunos Therapeutics, Inc., Fort Lauderdale, FL (US)

(21) Appl. No.: 19/166,924

(22) PCT Filed: Mar. 20, 2024
(86) PCT No.: PCT/US2024/020782

§ 371 Date: Sep. 19, 2025

Related U.S. Application Data
(60) Provisional application No. 63/453,676, filed on Mar. 21, 2023.

(51) Int. Cl. A61K 40/11; A61K 40/32; A61K 40/42; C12N 15/85

(52) U.S. Cl. CPC A61K 40/11; A61K 40/32; A61K 40/4201; C12N 15/85

(57) ABSTRACT

The present disclosure provides methods for identifying novel neoantigen-reactive T cell receptors (TCRs) by co-culturing a reporter T cell comprising a TCR expression cassette and an antigen presenting cell expressing a target neoantigen sequence and a matched human lymphocyte antigen (HLA) sequence. The present disclosure also provides novel neoantigen-reactive TCRs and the use thereof.

### CROSS-REFERENCE TO RELATED APPLICATION

This application claims the benefit of U.S. Provisional Application No. 63/453,676, filed Mar. 21, 2023, which is incorporated by reference in its entirety herein.

### INCORPORATION OF SEQUENCE LISTING

A sequence listing contained in the file named “P35358WO00_ST26.xml,” which is 417,107 bytes (measured in operating system MS-Windows) created on Mar. 20, 2024, is filed electronically herewith and incorporated by reference in its entirety.

### FIELD

The present disclosure relates to the identification of T cell receptors with defined antigen and HLA specificity and methods of using the same.

### BACKGROUND

Rapid and accurate identification of T cell receptors (TCRs) with defined antigen and HLA specificity has the potential to enable the discovery of TCRs with therapeutic applications. Individual T cell receptors (TCRs) can generally be defined with three key pieces of information; 1) Full-length paired (e.g., a and 3) TCR sequence, 2) Antigenic specificity and 3) HLA-restriction. Obtaining this information from a highly polyclonal population of T cells, such as those from peripheral blood or within tissues (e.g., tumor specimens) is challenging to do in an accurate and efficient manner.

At the intersection of cutting-edge technologies and robust immunological assay systems, a platform for overcoming this challenge has been developed and is provided in the present disclosure.

### SUMMARY

The present disclosure provides a neoantigen-reactive TCR, or an antigen-binding portion thereof, where the neoantigen-reactive TCR or the antigen-binding portion thereof can bind to a neoantigen/HLA complex, where the neoantigen comprises one of the EGFR L858R sequences provided in Table 7, and where the HLA is HLA-A*11:01.

The present disclosure provides a method for identifying a neoantigen-reactive TCR, comprising: i) co-culturing a) a reporter T cell comprising a TCR expression cassette, and b) an antigen presenting cell (APC) that expresses a target neoantigen sequence and a matched human leukocyte antigen (HLA) sequence; and ii) identifying a positive reporter signal in the reporter T cell to identify a neoantigen-reactive TCR. In one aspect, the methods disclosed herein comprises identifying TCR sequences from tumor infiltrating lymphocytes (TILs) isolated from a tumor sample. In another aspect, the methods further comprise identifying somatic mutations in the tumor sample and determining the germline HLA typing of the tumor sample.

The present disclosure provides a method of identifying a neoantigen-reactive T cell receptor (TCR), comprising: i) obtaining TCR α and β chain sequences from TILs isolated from a tumor sample; ii) obtaining neoantigen sequences comprising somatic mutations present in the tumor sample, and the germline HLA typing of the tumor sample; iii) co-culturing a) a reporter T cell expressing a TCR sequence reconstructed from the TCR α and β chain sequences obtained in step i), and b) an antigen presenting cell (APC) that expresses a neoantigen sequence and a matched human leukocyte antigen (HLA) sequence obtained in step ii); and iv) evaluating the reporter activity in the reporter T cell to identify a neoantigen-reactive TCR.

In one aspect, the present disclosure provides a method for identifying a neoantigen-reactive TCR, comprising: i) obtaining single-cell gene expression profiles from a population of tumor infiltrating lymphocytes (TIL) isolated from a patient sample, ii) performing bioinformatics analysis on the single cell gene expression data to identify TCR clonotypes, clustering the TCR clonotypes and to select a clonotype of interest, iii) creating recombinant alpha and beta TCR sequences in silico and preparing a reporter T cell comprising a TCR expression cassette encoding a TCR sequence reconstructed from paired TCR α and β chain sequences identified from the clonotype of interest in step ii), iv) preparing a tandem minigene (TMG) expression vector comprising nucleic acid sequences for the expression of concatenated amino acid sequences of non-synonymous single nucleotide variants (SNVs); v) analyzing the patient sequencing data to identify class I and class II HLA alleles and preparing HLA expression vectors comprising the class I HLA and class II HLA allele sequences; vi) preparing an APC comprising transfecting said TMG expression vector and up to four HLA expression vectors into a cell wherein each transfection condition comprises a TMG and one or two HLA types; vii) co-culturing the reporter T cell in step iii) with the APC of step vi), and viii) identifying a positive reporter activity in the reporter T cell to identify a neoantigen-reactive TCR. In certain aspect, the clustering comprises grouping the TCT clonotypes by CD8 or CD4 expression, gene function of differentially expressed genes, and the level of expression of each TCR.

In one aspect, the present disclosure provides a method for identifying a neoantigen-reactive TCR, comprising: i) obtaining single-cell gene expression profiles from a population of tumor infiltrating lymphocytes (TIL) isolated from a patient sample, ii) performing bioinformatics analyses on the single cell gene expression data to identify TCR clonotypes, clustering the TCR clonotypes and to select a clonotype of interest, iii) creating recombinant alpha and beta TCR sequences in silico and preparing a reporter T cell comprising a TCR expression cassette encoding a TCR sequence reconstructed from paired TCR α and β chain sequences identified from the clonotype of interest in step ii), iv) preparing a tandem minigene (TMG) expression vector comprising nucleic acid sequences for the expression of concatenated amino acid sequences of non-synonymous single nucleotide variants (SNVs); v) analyzing the patient sequencing data to identify class I and class II HLA alleles and preparing HLA expression vectors comprising the class I HLA and class II HLA allele sequences; vi) preparing an APC comprising transfecting said TMG expression vector and one or more HLA expression vectors into a cell wherein each transfection condition comprises a TMG and one or two HLA types; vii) co-culturing the reporter T cell in step iii) with the APC of step vi), and viii) identifying a positive reporter activity in the reporter T cell to identify a neoantigen-reactive TCR. In certain aspect, the clustering comprises grouping the TCT clonotypes by CD8 or CD4 expression, gene function of differentially expressed genes, and the level of expression of each TCR. In some aspects, the method comprises preparing an APC comprising transfecting the TMG expression vector and up to four, up to five, up to six, up to seven, up to eight, up to nine, up to ten, up to eleven, up to twelve, up to thirteen, up to fourteen, up to fifteen, up to sixteen, up to seventeen, up to eighteen, up to nineteen, or up to twenty HLA expression vectors into a cell. In one aspect, up to eight HLA expression vectors are transfected into a cell in the TCR screening protocol disclosed herein. In another aspect, up to seventeen HLA expression vectors are transfected into a cell in the TIL screening protocol disclosed herein. In some aspects, the method comprises pulsing neoantigen peptides into a cell instead of transfecting the cell with a TMG expression vector.

In a further aspect, the present disclosure provides a method for identifying a neoantigen-reactive TCR, comprising: i) obtaining single-cell gene expression profiles from a population of tumor infiltrating lymphocytes (TIL) isolated from a patient sample and whole exome sequence (WES) data from the patient sample, ii) performing bioinformatics analysis on the single cell gene expression data to identify TCR clonotypes of interest, iii) creating recombinant TCR sequences, iv) preparing a reporter T cell comprising a TCR expression cassette encoding a TCR sequence reconstructed from paired TCR α and β chain sequences identified from the clonotype of interest in step ii), v) preparing a tandem minigene (TMG) expression vector; vi) identifying class I and class II HLA alleles and preparing HLA expression vectors comprising the class I HLA and class II HLA allele sequences; vii) preparing an APC comprising transfecting said TMG expression vector and up to four HLA expression vectors into a cell wherein each transfection condition comprises a TMG and one or two HLA types; viii) co-culturing the reporter T cell in step iii) with the APC of step vi), and ix) identifying a positive reporter activity in the reporter T cell to identify a neoantigen-reactive TCR. In certain aspect, the clustering comprises grouping the TCT clonotypes by CD8 or CD4 expression, gene function of differentially expressed genes, and the level of expression of each TCR.

The present disclosure also provides a co-culture reporter system for identifying a T cell receptor (TCR) that recognizes a target neoantigen, comprising: i) a reporter T cell comprising a TCR expression cassette, co-cultured with ii) an antigen presenting cell (APC) that expresses a target neoantigen sequence and a matched human leukocyte antigen (HLA) sequence.

In one aspect, the TCR expression cassette as disclosed herein comprises a TCR sequence reconstructed from TCR α and β chain sequences identified from TILs isolated from a tumor sample, and wherein the target neoantigen sequence and the matched HLA sequence are identified from the same tumor sample. Methods of identifying TCR sequences, antigen or neoantigen sequences, or the HLA sequences from a tumor sample or a normal reference sample are known in the art. Some of the commonly used methods are also described herein.

In one aspect, the isolated TILs are first expanded ex vivo and then co-cultured with APCs modified to express relevant HLA alleles and antigens obtained from the tumor sample. In a further aspect, a gene signature for identifying neoantigen reactive TCRs from ex vivo expanded TILs includes one or more gene(s) selected from the group consisting of XCL2, XCL1, IL2, CSF2, IFNG, CCL4, CCL4L2, TNF, CCL3, RGCC, TNFSF9, DUSP2, NFKBID, MIR155HG, NR4A3, EVI2A, CRTAM, ZBED2, FABP5, PIM3, NR4A1, IL10, TNFSF14, NR4A2, LINC00892, ZFP36L1, GZMB, MYC, SPRY1, KDM6B, EGR2, PHLDA1, PPP1R2, VSIR, REL, PRDX1, SLA, CYTOR, DDX21, IER3, PGAM1, NAMPT, HSP90AB1, IL23A, FAM107B, BCL2A1, ZEB2, ZBTB32, BTG2, GADD45B, RILPL2, SEMA7A, TGIF1, SRGN, RAN, CFLAR, MAT2A, SIAH2, PRNP, RNF19A, FASLG, NME1, EVI2B, HSPH1, NOP16, CSRNP1, and TAGAP.

In one aspect, the reporter T cell disclosed herein is a primary T cell. In another aspect, the reporter T cell disclosed herein is from an immortalized T cell line. In a certain aspect, the reporter T cell disclosed herein is not a primary T cell. In certain aspects, the immortalized cell is a Jurkat cell or a SUP-T1 cell. In some aspects, the Jurkat cell is Jurkat NFAT. In one aspect, the endogenous T cell receptor of the cells is downregulated or knocked out, such as using routine methods in the art.

In one aspect, the reporter T cell disclosed herein expresses any or all protein components of the TCR signaling complex or downstream signaling components. In a certain aspect, the reporter T cell expresses one or more components selected from the group consisting of CD3, CD4, CD8a, and CD8b. In further aspects, these protein components are modified, such as by mutation of one or more amino acids, to enhance their activities.

In one aspect, the antigen presenting cell (APC) disclosed herein is a classical professional APC. In another aspect, the APCs disclosed herein are artificial APCs. In a certain aspect, the APC disclosed herein is not a professional APC. In certain aspects, the APC used in the methods or cell systems disclosed herein is a COS cell. In one aspect, the COS cell is a COS-7 cell. In one aspect, the APC is a 293-HEK cell. In another aspect, the APC is not a 293-HEK cell. In one aspect, the APC endogenously expresses an HLA allele. In another aspect, the APC does not express any endogenous HLA. In one aspect, the APC comprises one or more HLA expression plasmids. In one aspect, the APC expresses multiple HLA alleles in a single cell.

In one aspect, the APC expresses a co-stimulatory molecule. Examples of the co-stimulatory molecules include, but not limited to, 4-1BBL, CD40, CD80, CD86, or OX40L.

In some aspects, the reporter T cell disclosed herein comprises a reporter system that is activated by the binding of a TCR to an antigen. Examples of the reporter systems are known in the art and include, but are not limited to, systems based on luciferase activity, fluorescence, or cytokine production.

In one aspect of the present disclosure, the reporter T cells and the APCs are co-cultured at a ratio from about 16:1 to about 1:16. In one aspect, the reporter T cells and the APCs are co-cultured at a ratio of about 4:1. In another aspect, the reporter T cells and the APCs are co-cultured at a ratio of about 8:1. In certain aspect, the reporter T cells and the APCs are co-cultured at a ratio of about 1:16, 1:8, 1:4, 1:2, 1:1, 2:1, 4:1, 8:1, or 16:1.

In one aspect, the reporter T cells and the APCs are co-cultured for 1-48 hours. In another aspect, the reporter T cells and the APCs are co-cultured for about one hour, about 2 hours, about 3 hours, about hours, at least 5 hours, about 6 hours, about 7 hours, about 8 hours, about 9 hours, or about 10 hours.

The present disclosure provides TCR sequences, or an antigen-binding portion thereof, that are identified or obtained by any of the methods disclosed herein. In one aspect, a TCR sequence comprises one or more of the sequences selected from the group consisting of SEQ ID NOs:1-36 (the sequences provided in Tables 1-3). In another aspect, a TCR sequence comprises a sequence that is at least 80%, at least 85%, at least 90%, or at least 95% identical to a sequence selected from the group consisting of SEQ ID NOs:1-36 (the sequences provided in Tables 1-3).

The present disclosure provides a polynucleotide encoding an amino acid sequence that is at least 90%, 95%, 96%, 97%, 98%, 99%, or 100% identical to an amino acid sequence selected from the group consisting of SEQ ID NOs: 1-36 (the sequences provided in Tables 1-3).

The present disclosure also provides a neoantigen/HLA complex, where the neoantigen comprises a sequence selected from the group consisting of SEQ ID NOs: 67 to 292 and wherein the HLA comprises a sequence selected from a group consisting of SEQ ID NOs: 58 to 66. In one aspect, the neoantigen comprises a sequence selected from the sequences disclosed in Table 7.

The present disclosure also provides recombinant vectors expressing a TCR, or an antigen-binding portion thereof, that are disclosed herein. Production of recombinant vectors is well-known in the art, and a variety of vectors may be utilized, including viral or non-viral vectors.

In some aspects, the recombinant vector comprises a polycistronic expression cassette, wherein the polycistronic expression cassette comprises a transcriptional regulatory element operably linked to a polycistronic polynucleotide that comprises: a) a first polynucleotide sequence that encodes a T cell receptor (TCR) alpha chain comprising an alpha chain variable (Vα) region and an alpha chain constant (Cα) region; b) a second polynucleotide sequence that comprises a first 2A element; c) a third polynucleotide sequence that encodes a TCR beta chain comprising a beta chain variable (Vβ) region and a beta chain constant (Cβ) region; d) a fourth polynucleotide sequence that comprises a second 2A element; and e) a fifth polynucleotide sequence that encodes a fusion protein that comprises IL-15, or a functional fragment or functional variant thereof, and IL-15Rα, or a functional fragment or functional variant thereof. In one aspect, the recombinant vector o comprises the first, the second, the third, the fourth, and the fifth polynucleotide sequence in any order from 5′ to 3′. In some aspects, the TCR alpha chain comprises an amino acid sequence at least 90%, 91%, 92%, 93%, 94%, 95%, 96%, 97%, 98%, 99%, or 100% identical to the amino acid sequence of TCR alpha chain sequences disclosed in Tables 1-3, and the TCR beta chain comprises an amino acid sequence at least 90%, 91%, 92%, 93%, 94%, 95%, 96%, 97%, 98%, 99%, or 100% identical to the amino acid sequence of TCR beta chain sequences disclosed in Tables 1-3.

The present disclosure further provides a population of cells that comprise the recombinant vectors disclosed herein. In one aspect, the recombinant vector or the polynucleotide is integrated into the genome of the population of cells. In one aspect, the cells are immune effector cells. In certain aspects, the immune effector cells are selected from the group consisting of T cells, natural killer (NK) cells, B cells, mast cells, and myeloid-derived phagocytes.

The present disclosure provides a pharmaceutical composition comprising a population of cells as disclosed herein. In one aspect, the pharmaceutical composition comprises a pharmaceutically acceptable carrier.

The present disclosure further provides a method to treat or to prevent a medical condition, comprising administering a pharmaceutical composition described herein to a patient in need. In one aspect, the medical condition is a cancer.

### BRIEF DESCRIPTION OF THE DRAWINGS

FIG. 1 is a schematic of the TCR identification and screening platform.

FIG. 2 is a schematic showing the process for screening of TCRs obtained from TILs.

FIG. 3 is a schematic showing the comparison of TCR-based screening and TILs-based screening methods.

FIG. 4 presents the results of lentivirus infection of Jurkat NFAT cells to form CD8Lenti cells subsequently infected with CD4 lentivirus, as described in the Examples. Cells are harvested and stained with CD3, CD4, CD8A and CD8B. Jurkat NFAT parental cells are negative for CD8 (99.16% CD8 negative) on CD3+ cells. The results in FIG. 2 show that Jurkat LentiCD8 cells line have 43.57% CD8a expression and 43.56% CD8a and CD8b double positive expression.

FIG. 5 presents the results of single clones with high CD8 and luciferase activity signal to noise ratio. PBMCs from 3 different donors are irradiated and seeded in 96 multiwell plates at 100 k/well. Puromycin-selected Jurkat NFAT CD8Lenti stable pool cells are seeded at 0.5 cell/well on top of irradiated PBMCs (96 multiwells) to generate single clones. Single clones are cultured for 1 week with IL-2 (50IU/ml) and PHA (0.25 μg/ml). Second week cell medium is replaced with 100 IU/ml of IL-2. Grown back clones are evaluated for CD8a and CD8b expression and luciferase signal/noise ratio (PMA/Ionomycin vs untreated). FIG. 5 shows that clones #2, 15, 19, 41 (>95% CD8 expression and >150 signal to noise ratio) are the best clones with higher CD8 expression and higher luciferase activity signal to noise ratio.

FIG. 6 presents the results of flowcytometry analysis showing that cells from the Jurkat NFAT_CD8Lenti pool have 46.74% CD8A and CD8B double positive cells. Cells from the #41 clone show 95.74% CD8A and CD8B double positive cells.

FIG. 7 presents the results of flow analysis showing that cells from the Jurkat NFAT_CD8Lenti clone #41 infected with pGenLenti-CD4_IRES_Puro lentivirus have a CD4 positive population of 97.8%, compared to cells not infected.

FIG. 8 presents the results of single clones with high CD8 and luciferase activity signal to noise ratio. Jurkat cells are seeded in RPMI complete medium at 200 k cells/well in 96 multiwells. Cells are treated with PMA 50 ng/ml and Ionomycin 1 μg/ml for 2.5H, 3.5H, 4.5H and 5.5H. Cells are harvested and lysed with passive lysis buffer (Promega) at room temperature for 15 minutes. 50 μL of cell lysis are mixed with 100 ul of luciferase substrate (Promega). Luciferase signal intensities are detected with Luminometer. Luciferase activity folds changes are calculated by dividing PMA/Ionomycin treated condition to vehicle control treated conditions. FIG. 8 provides that 4-5 hours is the best time period to harvest cells since signals start to drop from CD8Lenti_CD4Lenti pool.

FIG. 9A presents the results of co-culturing Jurkat NFAT CD8Lenti cells with COS-7 cells transfected with TMG1 or TMG2 with 75 ng of HLA A*11:01 and 75 ng of HLA A*02:01. FIG. 9B presents TCR-mediated reporter activity in Jurkat NFAT cells expressing CD8 co-receptor.

FIG. 10 presents Jurkat NFAT cells with or without CD8 co-receptor electroporated with Curie, McClintock cells and stained with CD3, CD4, CD8a, CD8b and mTCR antibodies. Cells are analyzed using flowcytometry to detect the percentage of cells with mTCR expression. Cells are stained with CD3, CD4, CD8a, CD8b and mTCR antibodies. As shown, cells express similar level of mTCR in Jurkat NFAT CD8Lenti cells compared with Jurkat NFAT parental cells. Over 90% of cells are viable in all six cell lines on the next day after electroporation suggesting NEON electroporation system provides highly viable T cells with sufficient percentage of mTCR expression (~20%). This allows coculture experiments to be performed next day without wasting time to recover cells.

FIG. 11 presents flowcytometry analysis results for mTCR expression level in 11 TCRs for cells stained with CD3, CD4, CD8a, CD8b antibodies and mTCR antibodies. FIG. 11 shows that mTCR expression varied from 8-35% (9 of 11 TCRs expressed above 15%) when cells are gated on CD3+.

FIG. 12 presents the results of a luciferase activity, indicating the specificity of TMGs matched to 9 of 11 TCRs.

FIG. 13 presents the results of an experiment designed to troubleshoot samples with low TCR reactivity of FIG. 12. COS-7 cells are transfected with 75 ng of plasmids compared to the COS-7 cells transfected with 25 ng of plasmids in FIG. 9.

FIG. 14 presents results for peptide pulsing with long and short peptides and library TCRs.

FIG. 15 presents the results of the development of an anti-TCR positive control using Jurkat cells electroporated with various TCRs and H57 anti-TCR antibody coated multiwell plate.

FIG. 16 presents a scatter plot showing luciferase activity from anti-TCR positive control (FIG. 15, “Anti-TCR (Pos. Ctrl)”) vs. the percent expression of the electroporated TCR as measured by flow cytometry. A trend line (linear regression) is shown as a dotted line. The linear regression model and R² values are shown on the plot.

FIG. 17 shows expansion of TIL from Patient 0013 tumor fragments. Patient 0013 surgically resected tumor specimen is prepared to grow TILs. 24 fragments are harvested after 4 weeks. TILS are counted with NC3000 and total cell numbers are shown. T cell yield range from 1 million to 18 million.

FIG. 18 shows HLA allele plasmids and multiplex transfection for Patient 0013 TIL screening. Transfection is performed to express HLA plasmids in NeoAPCs. 4 groups of HLA are transfected in NeoAPCs. The HLA alleles transfected are specific to Patient 0013 germline HLA typing results.

FIG. 19 shows the mutations of the TMGs used in the screening process. TMG plasmids are co-transfected in NeoAPCs to express mutations identified in Patient 0013. There are total of 7 TMGs used in this screening process including the negative control plasmid (control TMG). Mutations in each TMG is shown in the table. Hotspot mutations including EGFR L858R and PIK3CA E545K are assembled in TMG-1.

FIG. 20 shows the combination of HLA and TMG used in the screening. For each of Patient 0013 TIL fragments, 24 different combination of HLA and TMG or peptide conditions are generated for screening including some controls such as HLA only or TIL only as negative control, and PMA/Ion as positive control.

FIG. 21 shows the IFN-7 ELISpot of co-cultured Patient 0013 TIL fragments with neoAPCs transfected with HLA Class I and TMG plasmids. ELISpot assay is performed to identify specific interaction between NeoAPCs being transfected HLA and mutations found in the Patient 0013 tumor and TILs grown from Patient 0013's tumor. The number of spots from ELISpot plates is counted and data is shown for class I HLAs. Fragments 6, 8, and 20 show higher number of SFUs against TMG-1 and topspot (TS) peptides.

FIG. 22 shows the IFN-7 ELISpot of co-cultured Patient 0013 TIL fragments with neoAPCs transfected with HLA Class I/II and TMG1 plasmid or TS peptides. ELISpot assay is performed to identify specific interaction between NeoAPCs transfected with HLA Class I and HLA Class II and mutations found in the Patient 0013 tumor and TILs grown from Patient 0013's tumor. Number of spots from ELISpot plates is counted and data are shown for both class I and class II HLAs. Fragments 1, 3, 6, 8, 9, 11, 14, 15, 18, 19, 20, 22, and 24 show higher SFUs against TMG-1 and/or topspot (TS) peptides.

FIG. 23 shows the IFN-7 ELISpot of co-cultured Patient 0013 TIL Fragments with neoAPCs Transfected with HLA Class II and Pulsed with TMG Peptide PoolsELISpot assay is performed to identify specific interaction between NeoAPCs being transfected HLA Class II and mutations found in the Patient 0013 tumor and TILs grown from Patient 0013's tumor. Number of spots from ELISpot plates is counted and data are shown for class II HLAs. No fragment is observed with substantial increase of spot numbers.

FIG. 24 shows 4-1BB expression on co-cultured Patient 0013 TIL fragments with neoAPCs transfected with HLA Class I and TMG plasmids. Cells in the ELISpot plate are collected and stained with antibodies against CD3, CD4, CD8 and 4-1BB. Percentage of 4-1BB positive cells in HLA class I transfected conditions are analyzed and shown on the figure. 4-1BB upregulation is consistently observed in fragment 6 and 18 against TMG-1 and topspot (TS) peptides.

FIG. 25 shows 4-1BB expression on co-cultured Patient 0013 TIL fragments with neoAPCs transfected with HLA Class I/II and TMG Plasmids. Cells in the ELISpot plate are collected and stained with antibodies against CD3, CD4, CD8 and 4-1BB. Percentage of 4-1BB positive cells in both HLA class I and II transfected conditions are analyzed. Fragments 4, 5, 6, 8, 13, 14, 17, 18, 19 and 21 show higher SFUs against TMG-1 and/or topspot (TS) peptides.

FIG. 26 shows 4-1BB expression on co-cultured Patient 0013 TIL fragments with neoAPCs transfected with HLA Class II and pulsed with TMG peptide pools. Cells in the ELISpot plate are collected and stained with antibodies against CD3, CD4, CD8 and 4-1BB. Percentage of 4-1BB positive cells in HLA class II transfected conditions are analyzed and shown on the figure. No fragment is observed with substantial increase of 4-1BB upregulation.

FIG. 27 shows flow cytometry scatter plots of CD8 (x-axis) and 4-1BB (y-axis) expression on Fragment 18 TIL after co-culture. Data demonstrates that the TIL reactivity is within the CD8+ TIL population.

FIG. 28 shows determination of antigen and HLA-allele reactivity of Patient 0013 TIL fragment 6 by 4-1BB expression. Patient 0013 TILs from fragment 6 are co-cultured with neoAPCs modified to express either HLA-A*11:01, HLA-C*03:04, or HLA-A*02:01 (negative control) and then pulsed either with control peptides, PIK3CA p.E545K or EGFR p.L858R peptides. After overnight co-culture TILs are harvested and 4-1BB expression is quantified in the CD8+ T cell gate.

FIG. 29 shows determination of antigen and HLA-allele reactivity of Patient 0013 TIL fragment 8 by 4-1BB expression. Patient 0013 TILs from fragment 8 are co-cultured with neoAPCs modified to express either HLA-A*11:01, HLA-C*03:04, or HLA-A*02:01 (negative control) and then pulsed either with control peptides, PIK3CA p.E545K or EGFR p.L858R peptides. After overnight co-culture TILs are harvested and 4-1BB expression is quantified in the CD8+ T cell gate.

FIG. 30 shows clustering of Patient 0013 TIL co-cultured with EGFRL858R expressing neoAPCs identifies activated T cells within Cluster 2. A UMAP of all cells display the relative proximity of the gene expression profile of cells in cluster 2 compared to the other clusters

FIG. 31 is another graph showing clustering of Patient 0013 TIL co-cultured with EGFRL858R expressing neoAPCs identifies activated T cells within Cluster 2. A heatmap of top differentially expressed genes between clusters show that cells in cluster 2 (FIG. 14) upregulate genes associated with T-Cell activation (CLL4L2, INFG, CCL4, XCL1, XCL2, RGCC, ZBED2, TNF, and CSF2) when compared with other clusters.

FIG. 32 shows Patient 0013 TCR clonotype 1 distribution in UMAP. Cells with Neoantigen reactive TCRs are identified after masking the dominant clonotype in Plotly interactive maps. The majority of expanded Tils are classified as clonotype 1.

FIG. 33 shows that Patient 0013 TCR Clonotypes 35, 102, and 319 are present in the activated T Cell Cluster 2. Cells with Neoantigen reactive TCRs are identified after masking the dominant clonotype in Plotly interactive maps. After hiding clonotype 1, three minor clonotypes C35, C102, and C319 found to have elevated expression of genes in our neoantigen reactive TIL signature and are also located in cluster 2.

FIG. 34 shows expression of Patient 0013 TCR1, TCR35, TCR102, and TCR319 in TCR-Reporter Cell Line. TCR Reporter cells are electroporated with TCR transposon expression vectors encoding TCR1, TCR 35, TCR102 or TCR319. The following day, transgenic TCR expression is measured by flow cytometry. The scatter plots show the mTCR expression (x-axis) for each of the TCRs. Control cells are not electroporated with any TCR vector. mTCR expression levels are shown as % gated on CD3-positive cells.

FIG. 35 shows that TCR-mediated reporter activity identifies EGFRL858R HLA-A*11:01 specific TCRs and minimal reactive epitope. NeoAPCs are transfected in each well with TMG HLA A*11:01. TCR reporter cells are electroporated with each of the TCR plasmid. Both wide type (WT) and mutant (MUT) peptide for EGFR L858R are pulsed on NeoAPCs. Three out of four TCRs are specific for EGFR L858R mutant peptide 9mer.

FIG. 36 presents flow cytometry plots showing expression of engineered TCRs in primary human T Cells in a representative donor. Primary human T cells are gene modified to stably express transgenic TCRs using Sleeping Beauty transposon/transposase gene transfer. After 25 days of ex vivo culture, cells are harvested and mTCR expression is measured. The contour plots show the data for a single representative donor. Cells are gated on total live lymphocytes.

FIG. 37 shows expression of engineers TCRs in primary human T cells from four independent donors. Primary human T cells from four independent donors are gene modified to stably express transgenic TCRs using Sleeping Beauty transposon/transposase gene transfer. After 25 days of ex vivo culture, cells are harvested and mTCR expression is measured. The mean and SEM of each TCR across the four donors is summarized in the plot.

FIG. 38 shows 4-1BB expression in CD4+ T Cells expressing engineered TCRs specific for EGFRL858R and HLA-A*11:01. TCR-T cells expressing TCR1, TCR319 or non-transposed T cells are co-cultured with monocyte-derived dendritic cells generated from an HLA-A*11:01 donor pulsed with EGFR L858 wild type or EGFR L858R mutant peptides. After overnight incubation, the cells are harvested and analyzed by flow cytometry for 4-1BB expression within the mTCR+CD4+ cell gate.

FIG. 39 shows 4-1BB expression in CD8+ T Cells expressing engineered TCRs specific for EGFRL858R and HLA-A*11:01.TCR-T cells expressing TCR1, TCR319 or non-transposed T cells are co-cultured with monocyte-derived dendritic cells generated from an HLA-A*11:01 donor pulsed with EGFR L858 wild type or EGFR L858R mutant peptides. After overnight incubation, the cells are harvested and analyzed by flow cytometry for 4-1BB expression within the mTCR+CD8+ cell gate.

FIG. 40 shows that TCR-T cell mediated cytolysis of tumor cells presenting either wild type EGFR or EGFRL858R antigen in the context of HLA-A*11:01. TCR-T cells expressing TCR1, TCR319 or non-transposed T cells are co-cultured with Saos2 tumor cells which have been modified to express HLA*A11:01 and are pulsed with EGFR L858 wild type or EGFR L858R mutant peptides. After overnight co-culture, target cell lysis is quantified using the CellTiter Glo assay. N=4 donors, statistical significance determined by 2-way ANOVA with Sidak correction for multiple comparisons, ****=p<0.0001.

### DETAILED DESCRIPTION

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the value or range remain within the intended meaning of the recited value or range.

As used herein, the terms “T cell receptor” and “TCR” are used interchangeably and refer to molecules comprising CDRs or variable regions from α3 T cell receptors. Examples of TCRs include, but are not limited to, full-length TCRs, antigen-binding fragments of TCRs, soluble TCRs lacking transmembrane and cytoplasmic regions, single-chain TCRs containing variable regions of TCRs attached by a flexible linker, TCR chains linked by an engineered disulfide bond, single TCR variable domains, single peptide-HLA-specific TCRs, multi-specific TCRs (including bispecific TCRs), TCR fusions, TCRs comprising co-stimulatory regions, human TCRs, humanized TCRs, chimeric TCRs, recombinantly produced TCRs, and synthetic TCRs. In certain aspects, the TCR is a full-length TCR comprising a full-length α chain and a full-length 3 chain. In certain aspects, the TCR is a soluble TCR lacking transmembrane and/or cytoplasmic region(s). In certain aspects, the TCR is a single-chain TCR (scTCR) comprising Vα and V3 linked by a peptide linker, such as a scTCR having a structure as described in PCT Publication No.: WO 2003/020763, WO 2004/033685, or WO 2011/044186, each of which is incorporated by reference herein in its entirety. In certain aspects, the TCR comprises a transmembrane region. In certain aspects, the TCR comprises a co-stimulatory signaling region.

As used herein, the term “full-length TCR” refers to a TCR comprising a dimer of a first and a second polypeptide chain, each of which comprises a TCR variable region and a TCR constant region comprising a TCR transmembrane region and a TCR cytoplasmic region. In certain aspects, the full-length TCR comprises one or two unmodified TCR chains, e.g., unmodified a or 3TCR chains. In certain aspects, the full-length TCR comprises one or two altered TCR chains, such as chimeric TCR chains and/or TCR chains comprising one or more amino acid substitutions, insertions, or deletions relative to an unmodified TCR chain. In certain aspects, the full-length TCR comprises a mature, full-length TCR α chain and a mature, full-length TCR β chain.

The “antigen-binding portion” of the TCR, as used herein, refers to any portion comprising contiguous amino acids of the TCR of which it is a part, provided that the antigen-binding portion specifically binds to the target neoantigen as described herein with respect to other aspects of the disclosure. The term “antigen-binding portion” refers to any part or fragment of the TCR of the disclosure, which part or fragment retains the biological activity of the TCR of which it is a part (the parent TCR). Antigen-binding portions encompass, for example, those parts of a TCR that retain the ability to specifically bind to the target antigen, or detect, treat, or prevent a condition, to a similar extent, the same extent, or to a higher extent, as compared to the parent TCR.

As used herein, the term “TCR variable region” refers to the portion of a mature TCR polypeptide chain (e.g., a TCR α chain or β chain) which is not encoded by the TRAC gene for TCR α chains, either the TRBC1 or TRBC2 genes for TCR β chains, or the TRDC gene for TCR δ chains. In some aspects, the TCR variable region of a TCR α chain encompasses all amino acids of a mature TCR α chain polypeptide which are encoded by a TRAV and/or TRAJ gene, and the TCR variable region of a TCR β chain encompasses all amino acids of a mature TCR β chain polypeptide which are encoded by a TRBV, TRBD, and/or TRBJ gene (see, e.g., Lefranc and Lefranc, (2001) “T cell receptor FactsBook.” Academic Press, ISBN 0-12-441352-8, which is incorporated by reference herein in its entirety). TCR variable regions generally comprise framework regions (FR) 1, 2, 3, and 4 and complementarity determining regions (CDR) 1, 2, and 3.

As used herein, the terms “α chain variable region” and “Vα” are used interchangeably and refer to the variable region of a TCR α chain.

As used herein, the terms “β chain variable region” and “Vβ” are used interchangeably and refer to the variable region of a TCR β chain.

As used herein in the context of a TCR, the term “CDR” or “complementarity determining region” means the noncontiguous antigen combining sites found within the variable regions of a TCR chain (e.g., an α chain or a β chain). These regions have been described in Lefranc, (1999) The Immunologist 7: 132-136; Lefranc et al., (1999) Nucleic Acids Res 27: 209¬212; Lefranc (2001) “T cell receptor FactsBook.” Academic Press, ISBN 0-12-441352-8; Lefranc et al., (2003) Dev Comp Immunol. 27(1):55-77; and in Kabat et al., (1991) “Sequences of protein of immunological interest,” each of which is herein incorporated by reference in its entirety. In certain aspects, CDRs are determined according to the IMGT numbering system described in Lefranc (1999) supra. In certain aspects, CDRs are defined according to the Kabat numbering system described in Kabat supra. In certain aspects, CDRs are defined empirically, e.g., based upon a structural analysis of the interaction of a TCR with a cognate antigen (e.g., a peptide or a peptide-HLA complex). In certain aspects, the α chain and β chain CDRs of a TCR are defined according to different conventions (e.g., according to the Kabat or IMGT numbering systems, or empirically based upon structural analysis).

As used herein, the term “constant region” with respect to a TCR refers to the portion of a TCR that is encoded by the TRAC gene (for TCR α chains) or either the TRBC1 or TRBC2 gene (for TCR 3 chains), optionally lacking all or a portion of a transmembrane region and/or all or a portion of a cytoplasmic region. In certain aspects, a TCR constant region lacks a transmembrane region and a cytoplasmic region. A TCR constant region does not include amino acids encoded by a TRAV, TRAJ, TRBV, TRBD, TRBJ, TRDV, TRDD, TRDJ, TRGV, or TRGJ gene (see, e.g., “T cell receptor Facts Book,” supra).

As used herein, the terms “major histocompatibility complex” and “MHC” are used interchangeably and refer to an MHC class I molecule and/or an MHC class II molecule.

As used herein, the term “MHC class I” refers to a dimer of an MHC class I α chain and a Beta-2 microglobulin chain and the term “MHC class II” refers to a dimer of an MHC class II α chain and an MHC class II β chain.

As used herein, the terms “human leukocyte antigen” and “HLA” are used interchangeably and can also refer to the proteins encoded by the MHC genes. HLA-A, HLA-B, HLA-C, HLA-E, HLA-F, and HLA-G refer to major and minor gene products of MHC class I genes. HLA-DP, HLA-DQ, and HLA-DR refer to gene products of MHC class I genes, which are expressed on antigen-presenting cells, B cells, and T cells.

As used herein, the term “peptide-HLA complex” refers to an HLA molecule (HLA class I, II or III) with a peptide bound in the art-recognized peptide binding pocket of the HLA. In some aspects, the HLA molecule is a membrane-bound protein expressed on the cell surface. In some aspects, the HLA molecule is a soluble protein lacking transmembrane or cytoplasmic regions.

Neoantigens are a class of cancer antigens which arise from cancer-specific mutations in expressed protein. As used herein, the term “neoantigen” relates to a peptide or protein expressed by a cancer cell that includes one or more amino acid modifications compared to the corresponding wild-type (non-mutated) peptide or protein that is expressed by a normal (non-cancerous) cell. A neoantigen may be patient specific. A “cancer-specific mutation” is a somatic mutation that is present in the nucleic acid of a tumor or cancer cell but absent in the nucleic acid of a corresponding normal, i.e., non-tumorous or non-cancerous, cell.

As used herein, the terms “T cell” and “T lymphocyte” are used interchangeably. In one aspect, the T cell is a primary T cell. In another aspect, the T cell is an immortalized T cell line. T cells can be obtained from numerous sources in a patient, including but not limited to tumor, blood, bone marrow, lymph node, the thymus, or other tissues or fluids. The T cells can include any type of T cell and can be of any developmental stage, including but not limited to, CD4+/CD8+ double positive T cells, CD4+ helper T cells, e.g., Th1 and Th2 cells, CD8+ T cells (e.g., cytotoxic T cells), tumor infiltrating cells (e.g., TILs), peripheral blood T cells, memory T cells, naive T cells, and the like. The T cells may be CD8+ T cells, CD4+ T cells, or both CD4+ and CD8+ T cells.

As used herein, the term “reporter T cell” refers to a T cell that comprises a TCR-mediated reporter system. Non-limiting examples of TCR-mediated reporter system include fluorescence-based systems, and those based on luciferase activity or cytokine production. See, e.g., Zong et al., 2020 PLoS ONE, and the references cited therein. A reporter system based on cytokine production may measure the production of one or more cytokines, the secretion of which by a T cell is characteristic of T cell activation (e.g., a TCR expressed by the T cells specifically binding to and immunologically recognizing the mutated amino acid sequence). Non-limiting examples of cytokines, the secretion of which is characteristic of T cell activation, include IFN-7, IL-2, granzyme B, and tumor necrosis factor α (TNF-α), granulocyte/monocyte colony stimulating factor (GM-CSF), IL-4, IL-5, IL-9, IL-10, IL-17, and IL-22. In certain aspect, a “positive” reporter signal in a reporter T cell is a signal from a reporter gene that is at least 1.5× higher than the average of all of the samples when measured in a 96 well plate having a single TCR, up to 6 TMG sequences in duplicate and five different HLA clusters. In aspects, the reporter signal is luciferase activity. A positive reporter signal is detected when the TCR in the reporter T cell is paired with a matching APC comprising a TMG and matched HLA cluster.

The phrase “neoantigen-reactive,” as used herein, means that a TCR, or an antigen-binding portion thereof, can bind to and immunologically recognize the mutated amino acid sequence encoded by the cancer-specific mutation.

As used herein, the terms “treat,” “treating,” and “treatment” refer to therapeutic or preventative measures described herein. In some aspects, the methods of “treatment” employ administration of a TCR or a cell expressing a TCR to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “subject” includes any human or non-human animal. In one aspect, the subject is a human or non-human mammal. In one aspect, the subject is a human. As used herein, the term “polycistronic vector” refers to a polynucleotide vector that comprises a polycistronic expression cassette.

As used herein, the term “polycistronic expression cassette” refers to a polynucleotide sequence wherein the expression of three or more transgenes is regulated by common transcriptional regulatory elements (e.g., a common promoter) and can simultaneously express three or more separate proteins from the same mRNA. Exemplary polycistronic vectors, without limitation, include tricistronic vectors (containing three cistrons) and tetracistronic vectors (containing four cistrons).

As used herein, the term “polycistronic polynucleotide” refers to a polynucleotide that comprises three or more cistrons.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87: 2264-2268, modified as in Karlin S & Altschul S F, (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package.

The percent identity between two sequences can be determined using techniques similar to those described above, with or without allowing gaps. In calculating percent identity, typically only exact matches are counted.

The present disclosure provides a method for identifying a TCR that recognize a target neoantigen, comprising: i) co-culturing a) a reporter T cell comprising a TCR expression cassette, and b) an antigen presenting cell (APC) that expresses a target neoantigen sequence and a matched human leukocyte antigen (HLA) sequence; and ii) evaluating the reporter activity in the reporter T cell to identify a TCR that recognizes the target neoantigen. In one aspect, the methods disclosed herein comprises identifying TCR sequences from tumor infiltrating lymphocytes TILs isolated from a tumor sample. In another aspect, the methods further comprise identifying somatic mutations in the tumor sample and determining the germline HLA typing of the tumor sample.

The present disclosure provides a method of identifying a neoantigen-reactive T cell receptor (TCR), comprising: i) obtaining TCR α and β chain sequences from TILs isolated from a tumor sample; ii) obtaining neoantigen sequences comprising somatic mutations present in the tumor sample, and the germline HLA typing of the tumor sample; iii) co-culturing a) a reporter T cell expressing a TCR sequence reconstructed from the TCR α and β chain sequences obtained in step i), and b) an antigen presenting cell (APC) that expresses a neoantigen sequence and a matched human leukocyte antigen (HLA) sequence obtained in step ii); and iv) evaluating the reporter activity in the reporter T cell to identify a neoantigen-reactive TCR.

The present disclosure also provides a co-culture reporter system for identifying a T cell receptor (TCR) that recognizes a target neoantigen, comprising: i) a reporter T cell comprising a TCR expression cassette, co-cultured with ii) an antigen presenting cell (APC) that expresses a target neoantigen sequence and a matched human leukocyte antigen (HLA) sequence.

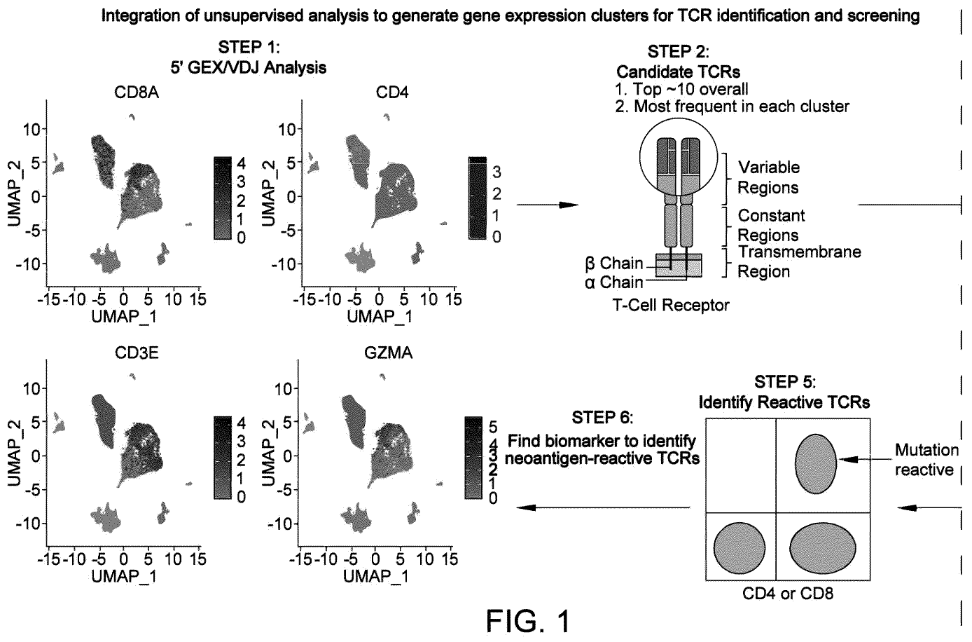

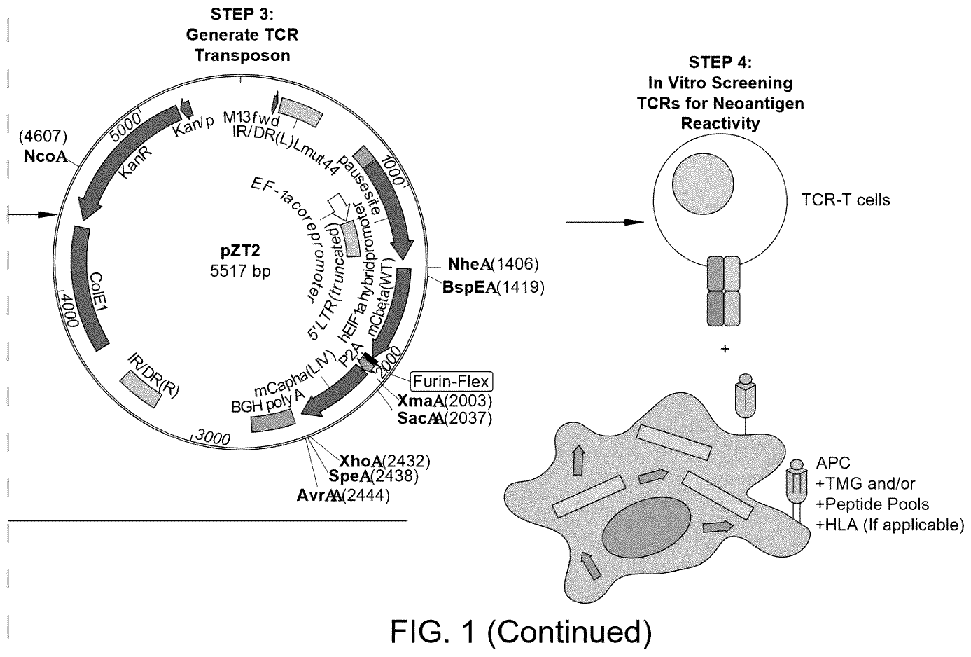

The present disclosure provides a TCR identification and screening platform as illustrated in FIG. 1. Initially, single-cell gene expression data (e.g., 5′ GEX Analysis) from T cells are utilized to perform unsupervised clustering analysis by employing dimensionality reduction methods such as principal component analysis (PCA), t-distributed Stochastic Neighbor Embedding (tSNE), or Uniform Manifold Approximation and Projection (UMAP) (FIG. 1, STEP 1). Merging the clustered single-cell gene expression analysis with paired, full-length TCR sequences then enables the identification of TCR clonotypes present in each of the distinct clusters. TCR sequences are then selected from the overall single-cell dataset based on frequency, cluster attributes, specific-gene expression signatures, or other criteria employed to increase the likelihood of obtaining TCRs with desired reactivity (i.e., antigen/HLA specificity) (FIG. 1, STEP 2). Selected paired, full-length TCR sequences are then reconstructed in silico, from which expression plasmids encoding the TCR α and R chains are synthesized (FIG. 1, STEP 3). These TCR expression cassettes are then cloned into transposon or other non-viral gene transfer vectors to enable quick translation into process development, manufacturing, and clinical applications. TCR-expression plasmids are then transiently expressed in a cell line (e.g., Jurkat or SUP-T1) or primary cell (e.g., human ex vivo expanded T cells) that will signal upon TCR recognition of cognate antigen:HLA complexes on the surface of antigen presenting cells (APCs) (FIG. 1, STEP 4). Antigen presenting cells (APCs) are classical professional APCs such as dendritic cells (DCs) or an artificial antigen presenting cell (e.g., COS-7 or 293-HEK). APCs either endogenously express the requisite HLA allele(s) or are transfected with HLA expression plasmids. Antigens are introduced to the APCs either by genetic transfer to antigen encoding plasmids (e.g., Tandem Minigene (TMG) plasmids) or by the pulsing of peptide pools. One aspect of the APC system used is that multiple HLA alleles and antigens are screened within the same set of APCs, thus enable high-throughput assessment of hundreds to thousands of antigen:HLA combinations. Co-culture of the TCR modified cells and APCs is then performed to identify reactive TCRs (FIG. 1, STEP 5). Reactive TCRs are those that are found to recognize one of the antigen:HLA conditions tested. These reactive TCRs are then further evaluated in vitro to confirm the findings and deconvolute the multiplexed HLA/antigen. Once all reactive TCRs are identified from a specimen, that binary outcome (reactive vs non-reactive) for each TCR can be mapped back to the initial gene-expression cluster analysis (FIG. 1, STEP 6). By mapping the reactive TCRs back to the gene-expression data, gene signatures or biomarkers which are enriched in the reactive TCR cell population are elucidated and used to further improve and refine the initial selection of TCRs for screening. In one aspect, this process is used to identify TCR sequences and their associated antigen and HLA specificity with a high level of confidence and accuracy from complex starting materials such as tumor tissues or blood samples.

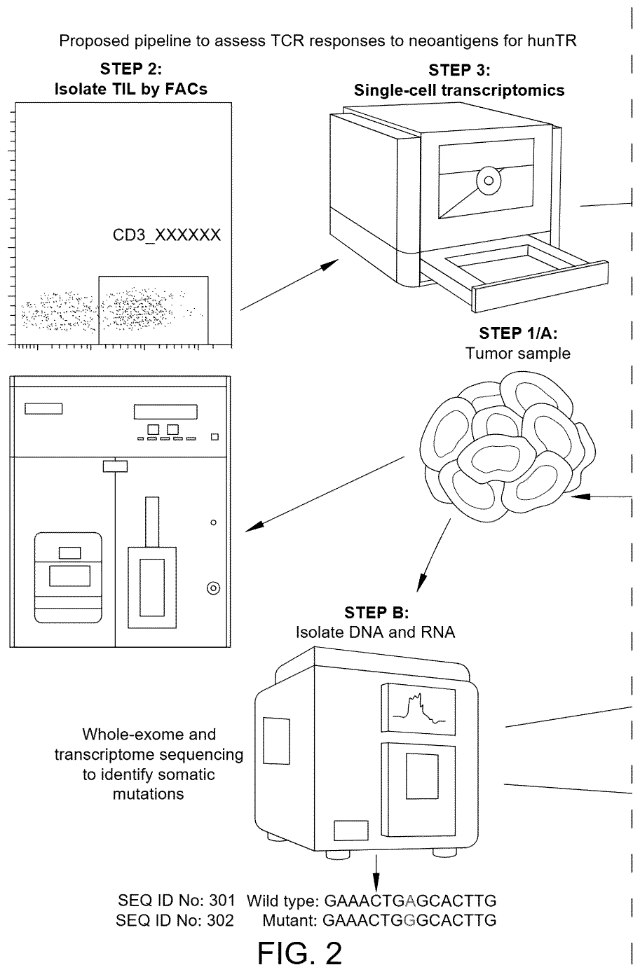

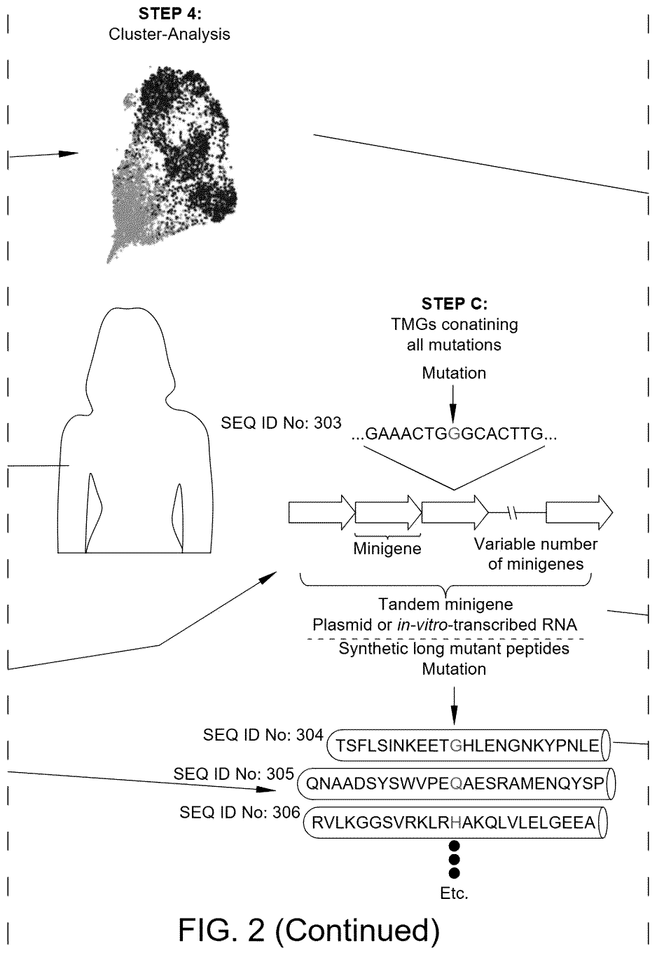

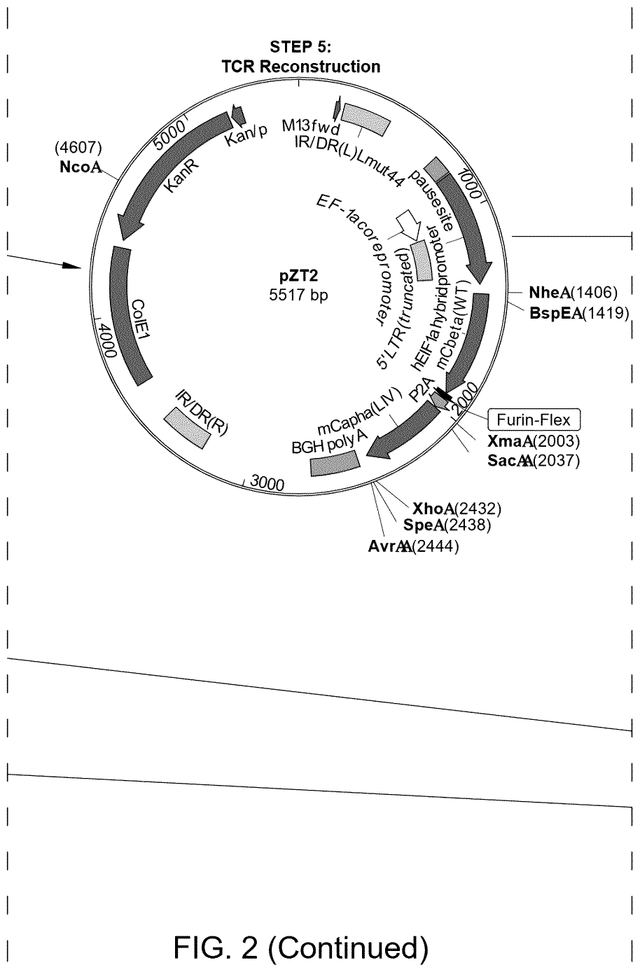

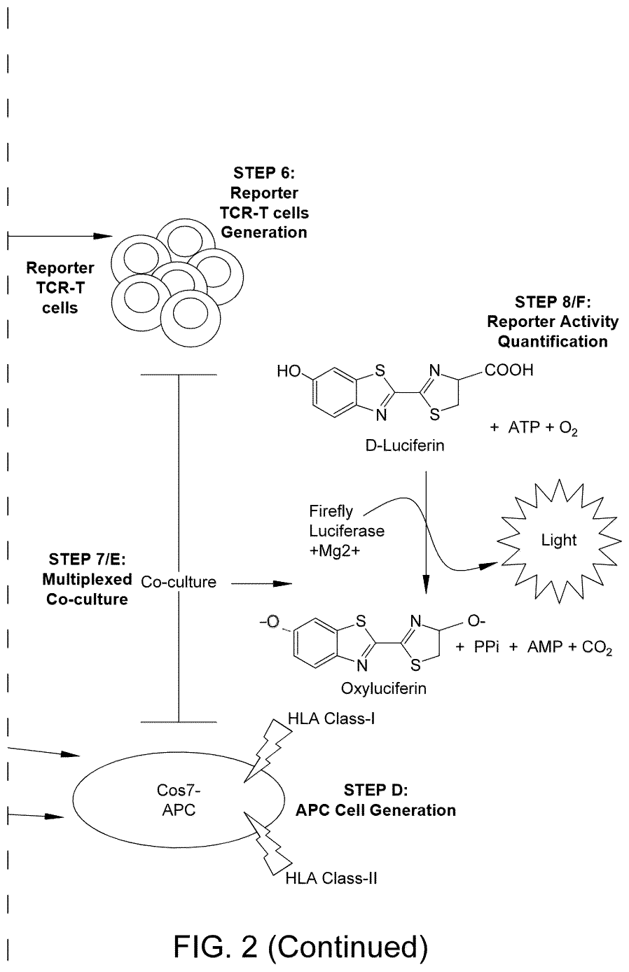

In one aspect, the steps of the above-described workflow (FIG. 1) comprise the processes as shown in FIG. 2 for screening of TCRs obtained from TILs. The process illustrated in FIG. 2 correspond to FIG. 1 STEPs 1-5. The workflow illustrated in FIG. 2 further comprises two parallel processes (indicated with either Alpha [i.e., A, B, C, etc.] or Numeric [i.e., 1, 2, 3, etc.] STEP designators) that diverge from a common starting point (STEP 1/A) and converge at a common finishing point (STEP 8/F). STEP 1/A to STEP 6 illustrate the workflow from TILs isolation to generation of cells expressing TILs-derived TCRs. STEP 1/A to STEP D illustrate the workflow from patient mutation and HLA calling to the generation of APCs expressing the patient matched HLA and mutation-derived antigens (e.g., neoantigens).

In one aspect, a tumor sample is obtained from a cancer patient (FIG. 2, STEP 1/A). This tumor sample is dissociated into a single-cell suspension and TILs are isolated by fluorescent activated cell sorting (FACS) by staining dissociated tumor samples for lymphocyte, T cell, and live cell markers (FIG. 2, STEP 2). Single-cell transcriptomics is then performed on the sorted TILs to obtain gene expression and TCR V(D)J sequences (FIG. 2, STEP 3). Bioinformatic analysis of the gene-expression data is used to cluster cells based on transcriptional similarities to aid in the selection of TCR sequences for in vitro evaluation (FIG. 2, STEP 4). Once selected, TCRs are reconstructed in silico and synthesized in expression vectors (FIG. 2, STEP 5) to enable transgenic expression of the TCRs in cells capable of forming a functional TCR complex with CD3 subunits and CD4/CD8 co-receptors. These cells are engineered to express any or all necessary protein components of the TCR signaling complex or downstream signaling components. Moreover, these components are modified to further enhance their function in the platform (e.g., CD4 with amino acid substitutions at Q40Y, T45W, P48L, S60R, and/or D63R to enhance affinity to MHC-Class II). Wang et al. 2011 PNAS, 108(38): 15960-15965. TCR expression vectors are transferred into the reporter cells to generate reporter TCR-T cells (FIG. 2 STEP 6).

In another aspect, in parallel to STEPs 1-6 described above, nucleic acids (DNA and RNA) are extracted from the tumor sample (FIG. 2, STEP 1/A). Using Whole Exome Sequencing (WES) and RNA Sequencing (RNAseq) to generate genomic and transcriptional datasets, a bioinformatics pipeline is employed to determine somatic mutations present in the tumor as well as the patient's germline HLA typing (FIG. 2, STEP B). Somatic mutations are ranked and concatenated so that TMGs and peptide pools can be synthesized (FIG. 2, STEP C). These reagents provide the antigen component of the screening assay. Similarly, sequences of the called HLA alleles are synthesized in expression vectors to provide the HLAs necessary for the screening assay. Antigen presenting cells, such as COS-7, are then modified either by stable or transient transfection to express the requisite Class I or Class II HLA alleles either in single-plex or multiplexed within the same cells (FIG. 2, STEP D). Antigen is provided to the APCs either by transfection of relevant TMGs (either as plasmid DNA or in vitro transcribed RNA) and/or peptide pools containing antigens derived from the tumor's somatic mutations identified. With both the HLA and antigen provided to the APCs, they are able to present peptide:HLA complexes to T cells in vitro.

In a further aspect, reporter cells expressing transgenic TCRs (FIG. 2, STEP 6) and antigen/HLA-modified APCs (FIG. 2, STEP D) are co-cultured together at a pre-determined ratio of Reporter cells (E) to APCs (T), typically approximately 4:1 to 8:1 (FIG. 2, STEP 7/E). Positive control wells containing PMA/Ionomycin or coated with H57-597 antibody (anti-transgenic TCR) with the TCR-modified Reporter cells are also set up. Negative control wells of Reporter cells alone or co-cultured with APCs modified with HLA-only, irrelevant antigens, or non-transfected are also set up. All conditions are typically evaluated in duplicate. After the co-culture period, reporter activity (i.e., luciferase activity) is quantified in each co-culture and control well (FIG. 2, STEP 8/F). For a given TCR, the reporter activity is compared across all antigen:HLA conditions evaluated to determine if there is a condition with increased reporter activity which indicates that the transgenic TCR recognized an antigen:HLA combination present in that well. Because initial screening multiplexes multiple HLA alleles and antigens, when there is specific TCR activity observed, STEP 7/E and 8/F are repeated using APCs modified with single HLA and antigens to elucidate the exact specificity of the TCR. Moreover, minimal epitopes can be determined using this co-culture method. Overall, this workflow enables the identification of TCR sequences and the empirical determination of specificity to selected antigens and HLA alleles.

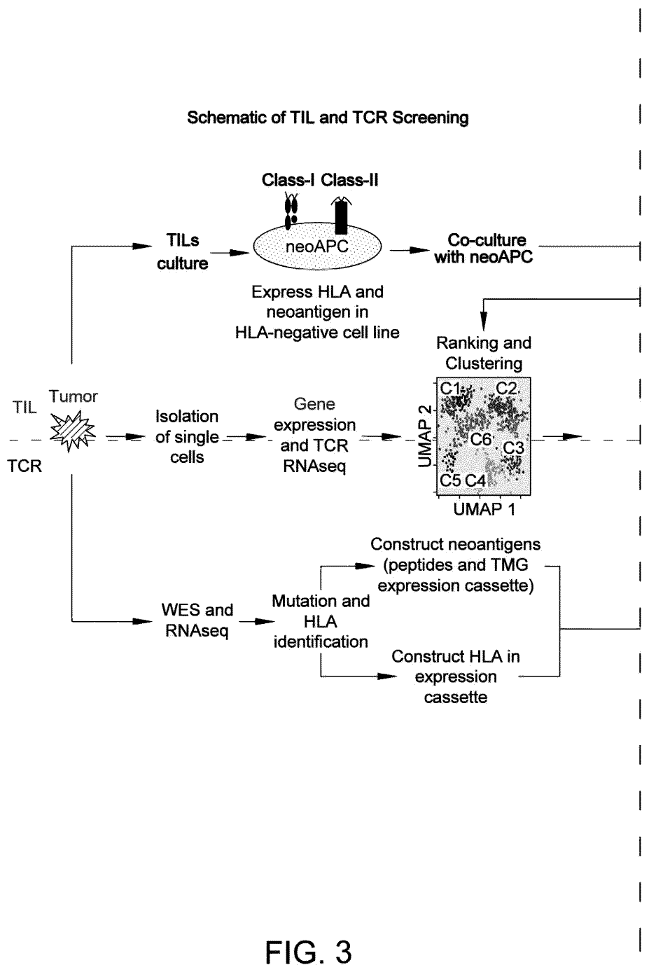

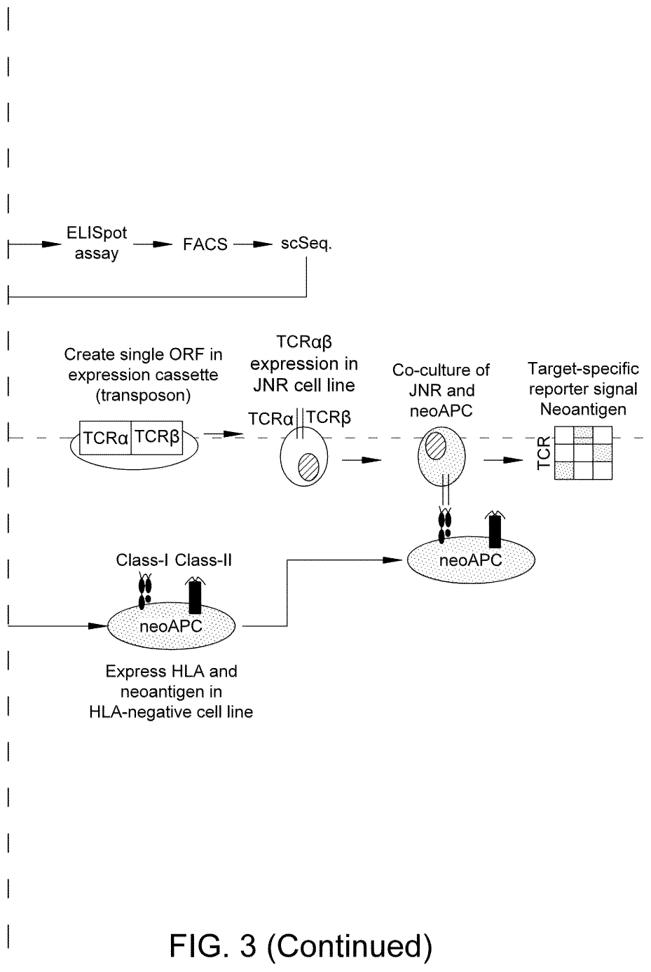

The present disclosure provides both a TCR-based screening method (below dotted line) and a TILs-based screening method (above dotted line), as illustrated in FIG. 3. The TCR-based screening method is as described above in the description of FIG. 2 wherein TCR sequences, somatic mutations, and HLA-typing is obtained from primary tumor samples and utilized to screen selected TCRs for reactivity to tumor neoantigens using a co-culture reporter system. Similarly, TILs screening starts with a primary tumor sample obtained from a cancer patient. TILs are expanded from the tumor using standard TILs expansion methods (high-concentration IL-2, feeder cells, muromonab-CD3 (OKT3)). Expanded TILs are then co-cultured in an IFN-γ ELISpot with APCs modified to express the relevant HLA alleles and antigens identified from WES and RNAseq data from the tumor. This is performed in a similar plate layout to TCR screening where multiple HLA alleles and antigens are multiplexed in the same wells, thus increasing the throughput of the assay. Positive controls include PMA/Ionomycin. Negative controls include TILs alone, APCs alone, TILs+APCs without HLA and/or antigen, and no cells. After the overnight co-culture, cells are harvested from the IFN-γ ELISpot and the plate is developed to measure the number of spot-forming colonies (SFCs) of each well. The harvested TILs are also stained and evaluated for upregulation of 4-1BB or other activation molecules (e.g., OX40). TILs from co-culture conditions which produce increased numbers of SFCs and/or activation marker expression are then sorted for either total live T cells or for T cells expressing the activation marker. Single cell gene expression and TCR V(D)J sequencing is then performed on the sorted cells. T cells from a negative control co-culture (typically APCs modified with HLA alone or with HLA and irrelevant antigen) are similarly sorted and analyzed by single-cell transcriptomics. Using the single-cell gene expression data, clusters of activated TILs can be identified. Paired, full-length TCR sequences from these activation clusters are then reconstructed into TCR expression plasmids and screened using the TCR screening methods described in FIG. 2. Overall, FIG. 3 illustrates parallel workflows with either ex vivo expanded TILs or sorted TILs are utilized to identify tumor-reactive TCRs with potential therapeutic applications in oncology. These general methods are applied to identify therapeutically useful TCRs in other disease indications (e.g., inflammation, auto-immune, etc.) with the appropriate starting material (e.g., a biopsy of inflamed colon from Crohn's disease patient or a plaque of a patient with psoriasis).

In one aspect, the cells in the methods or systems described herein are mammal cells, such as human cell, mouse cell, or monkey cells. In another aspect, the cells in the methods or systems described herein are non-human primate cells. In one aspect, the reporter T cells and the APCs are from different species.

In one aspect, the TCR expression cassette as disclosed herein comprises a TCR sequence reconstructed from TCR α and β chain sequences identified from TILs isolated from a tumor sample, and wherein the target neoantigen sequence and the matched HLA sequence are identified from the same tumor sample. Methods of identifying TCR sequences, antigen or neoantigen sequences, or the HLA sequences from a tumor sample or a normal reference sample are known in the art. Non-limiting examples of some commonly used methods are also disclosed herein. In one aspect, the TCR expression cassette is cloned into a non-viral gene transfer vector. In another aspect, the TCR expression cassette is cloned into a viral gene transfer vector. In a particular aspect, the non-viral gene transfer vector is a transposon.

In one aspect, the isolated TILs are first expanded ex vivo and then co-cultured with APCs modified to express relevant HLA alleles and antigens obtained from the tumor sample. In a further aspect, a gene signature for identifying neoantigen reactive TCRs from ex vivo expanded TILs includes one or more gene(s) selected from the group consisting of XCL2, XCL1, IL2, CSF2, IFNG, CCL4, CCL4L2, TNF, CCL3, RGCC, TNFSF9, DUSP2, NFKBID, MIR155HG, NR4A3, EVI2A, CRTAM, ZBED2, FABP5, PIM3, NR4A1, IL10, TNFSF14, NR4A2, LINC00892, ZFP36L1, GZMB, MYC, SPRY1, KDM6B, EGR2, PHLDA1, PPP1R2, VSIR, REL, PRDX1, SLA, CYTOR, DDX21, IER3, PGAM1, NAMPT, HSP90AB1, IL23A, FAM107B, BCL2A1, ZEB2, ZBTB32, BTG2, GADD45B, RILPL2, SEMA7A, TGIF1, SRGN, RAN, CFLAR, MAT2A, SIAH2, PRNP, RNF19A, FASLG, NME1, EVI2B, HSPH1, NOP16, CSRNP1, and TAGAP.

In one aspect, the reporter T cell disclosed herein is a primary T cell. In another aspect, the reporter T cell disclosed herein is from an immortalized T cell line. In a certain aspect, the reporter T cell disclosed herein is not a primary T cell. In certain aspects, the immortalized cell is a Jurkat cell or a SUP-T1 cell. In some aspects, the Jurkat cell is Jurkat NFAT. In one aspect, the endogenous T cell receptor of the cells is downregulated or knocked out, such as using routine methods in the art.

In one aspect, the reporter T cell disclosed herein expresses any or all protein components of the TCR signaling complex or downstream signaling components. In a certain aspect, the reporter T cell expresses one or more components selected from the group consisting of CD3, CD4, CD8a, and CD8b. In further aspects, these protein components are modified, such as by mutation of one or more amino acids, to enhance their activities.

In one aspect, the antigen presenting cell (APC) disclosed herein is a classical professional APC. In another aspect, the APCs disclosed herein are artificial APCs. In one aspect, the APC described herein does not express an endogenous human HLA. An endogenous human HLA may be knocked out from an APC by methods known in the art, e.g., CRISPR. In a further aspect, the APC comprises the machinery for antigen presentation still and be amenable to modification by transient or stable transgene expression of HLAs. In another aspect, the APC is modified with human beta-2-microglobulin, human CLIP, human TAP1 or TAP2, or any other human-derived molecular components of antigen processing and presentation. In a certain aspect, the APC disclosed herein is not a professional APC. In certain aspects, the APC used in the methods or cell systems disclosed herein is a COS cell. In one aspect, the COS cell is a COS-7 cell. In one aspect, the APC is a 293-HEK cell. In another aspect, the APC is not a 293-HEK cell. In one aspect, the APC endogenously expresses an HLA allele. In another aspect, the APC does not express any endogenous HLA. In one aspect, the APC comprises one or more HLA expression plasmids. In one aspect, the APC expresses multiple HLA alleles in a single cell.

In one aspect, the APC expresses a co-stimulatory molecule. Examples of the co-stimulatory molecules include, but not limited to, 4-1BBL, CD40, CD80, CD86, or OX40L.

In one aspect, antigen or neoantigen sequences are introduced to the APCs either by genetic transfer to antigen encoding plasmids (e.g., Tandem Minigene (TMG) plasmids) or by the pulsing of peptide pools. A Tandem Minigene is an open reading frame comprising concatenated minigenes which encode about 25 aa each. The minigenes encode the mutated region of the gene as identified from sequencing (typically 12 aa upstream and downstream of the substituted aa residue). These minigenes are flanked at the 5′ end with a LAMP1 signal peptide and 3′ end DC-LAMP localization signal. One aspect of the APC system used is that multiple HLA alleles and antigens are screened within the same set of APCs, thus enable high-throughput assessment of hundreds to thousands of antigen:HLA combinations. In one aspect, a “matched” HLA sequence of a neoantigen sequence refers to an HLA sequence that is identified from tissue, blood, or tumor samples of the same patient as the TCR sequence and neoantigen sequence. In certain aspect, “matched” HLA sequence may also be used to indicate the HLA sequence of the HLA allele for which a particular TCR is restricted.

In some aspects, the reporter T cell disclosed herein comprises a reporter system that is activated by the binding of a TCR to an antigen. Examples of the reporter systems are known in the art and include, but are not limited to, systems based on luciferase activity, fluorescence, or cytokine production.

In one aspect of the present disclosure, the reporter T cells and the APCs are co-cultured at a ratio from about 16:1 to about 1:16. In one aspect, the reporter T cells and the APCs are co-cultured at a ratio of about 4:1. In another aspect, the reporter T cells and the APCs are co-cultured at a ratio of about 8:1.

In one aspect, the reporter T cells and the APCs are co-cultured for 1 to 48 hours. In one aspect, the reporter T cells and the APCs are co-cultured for at least one hour, at least 2 hours, at least 3 hours, at least 4 hours, at least 5 hours, at least 6 hours, at least 7 hours, at least 8 hours, at least 9 hours, or at least 10 hours. In another aspect, the reporter T cells and the APCs are co-cultured for about one hour, about 2 hours, about 3 hours, about hours, at least 5 hours, about 6 hours, about 7 hours, about 8 hours, about 9 hours, or about 10 hours.

In one aspect, the TCRs disclosed herein interacts with and/or is specific for a peptide from a gene selected from a group comprising KRAS, RHPN2, GFRA2, NUP205, PCSK9, CEP85, HNRNPF, KDM1A, USP9X, LLGL1, ACO2, POLDIP3, EMC8, LCK, RCC1, VARS, LCK, ATP1A1, and CRYBG3.

The present disclosure provides TCR sequences, or an antigen-binding portion thereof, that are identified or obtained by any of the methods disclosed herein. In one aspect, a TCR sequence comprises one or more of the sequences selected from the group consisting of SEQ ID NOs: 1-36 (the sequences provided in Tables 1-3). In another aspect, a TCR sequence comprises a sequence that is at least 80%, at least 85%, at least 90%, or at least 95% identical to a sequence selected from the group consisting of SEQ ID NOs: 1-36 (the sequences provided in Tables 1-3).

In Tables 1 to 3, all of the sequences are fully human except for the “α chain with WT signal peptide and constant Cα” and “β chain with WT signal peptide and constant Cβ.” The sequences in these two sections are chimeric, containing the variable region sequences of the human TCRs combined with the constant region sequences of murine α and β chains.

TABLE 1

| SEQ ID NO. | Description | 0013-TCR1 |
|---|---|---|
| 1 | CDR1α | DRGSQS |
| 2 | CDR2α | IYSNGD |
| 3 | CDR3α | AVYNNNDMR |
| 4 | Vα without signal | QQKEVEQNSGPLSVPEGAIASLNCTYSDRGSQSFF |
| | peptide (SignalP) | WYRQYSGKSPELIMSIYSNGDKEDGRFTAQLNKAS |
| | | QYVSLLIRDSQPSDSATYLCAVYNNNDMRFGAGTR |
| | | LTVKPNIQNPEPAV |
| 5 | Vα only (without | MMKSLRVLLVILWLQLSWVWSQQKEVEQNSGPLS |
| | the Constant) | VPEGAIASLNCTYSDRGSQSFFWYRQYSGKSPELIM |
| | | SIYSNGDKEDGRFTAQLNKASQYVSLLIRDSQPSDS |
| | | ATYLCAVYNNNDMRFGAGTRLTVKPNIQNPEPAV |
| 6 | α chain with WT | MMKSLRVLLVILWLQLSWVWSQQKEVEQNSGPLS |
| | signal peptide and | VPEGAIASLNCTYSDRGSQSFFWYRQYSGKSPELIM |
| | constant Cα | SIYSNGDKEDGRFTAQLNKASQYVSLLIRDSQPSDS |
| | | ATYLCAVYNNNDMRFGAGTRLTVKPNIQNPEPAV |
| | | NIQNPEPAVYQLKDPRSQDSTLCLFTDFDSQINVPK |
| | | TMESGTFITDKTVLDMKAMDSKSNGAIAWSNQTSF |
| | | TCQDIFKETNATYPSSDVPCDATLTEKSFETDMNLN |
| | | FQNLLVIVLRILLLKVAGENLLMTLRLWSS |
| 7 | CDR1β | SGHTA |
| 8 | CDR2β | FQGTGA |
| 9 | CDR3β | ASSFLAGHLYTGELF |
| 10 | Vβ without signal | GVSQTPSNKVTEKGKYVELRCDPISGHTALYWYRQ |
| | peptide (SignalP) | SLGQGPEFLIYFQGTGAADDSGLPNDRFFAVRPEGS |
| | | VSTLKIQRTERGDSAVYLCASSFLAGHLYTGELFFG |
| | | EGSRLTVLEDLRN |
| 11 | Vβ (without the | MGTRLLCWAALCLLGADHTGAGVSQTPSNKVTEK |
| | Constant) | GKYVELRCDPISGHTALYWYRQSLGQGPEFLIYFQ |
| | | GTGAADDSGLPNDRFFAVRPEGSVSTLKIQRTERG |
| | | DSAVYLCASSFLAGHLYTGELFFGEGSRLTVLEDLR |
| | | N |
| 12 | β chain with WT | MGTRLLCWAALCLLGADHTGAGVSQTPSNKVTEK |
| | signal peptide and | GKYVELRCDPISGHTALYWYRQSLGQGPEFLIYFQ |
| | constant Cβ | GTGAADDSGLPNDRFFAVRPEGSVSTLKIQRTERG |
| | | DSAVYLCASSFLAGHLYTGELFFGEGSRLTVLEDLR |
| | | NEDLRNVTPPKVSLFEPSKAEIANKQKATLVCLAR |
| | | GFFPDHVELSWWVNGKEVHSGVSTDPQAYKESNY |
| | | SYCLSSRLRVSATFWHNPRNHFRCQVQFHGLSEED |
| | | KWPEGSPKPVTQNISAEAWGRADCGITSASYQQGV |
| | | LSATILYEILLGKATLYAVLVSTLVVMAMVKRKNS |

In some aspects, 0013-TCR1 interacts with and/or is specific for a peptide from gene EGFR. In some aspects, the peptide is from a neoantigen of EGFR and has the amino acid change L858R (in which position 858 of the EGFR protein is mutated from Len to Arg). In some aspects, 0013-TCR1 interacts with and/or is specific for the neoantigen in the context of HLA-A*11:01.

TABLE 2

| SEQ ID NO. | Description | 0013-TCR102 |
|---|---|---|
| 13 | CDR1α | NSASDY |
| 14 | CDR2α | IRSNMDK |
| 15 | CDR3α | AETPDKLI |
| 16 | Vα without signal | VGLHLPTLSVQEGDNSIINCAYSNSASDYFIWYKQESGKGPQFII |
| | peptide (SignalP) | DIRSNMDKRQGQRVTVLLNKTVKHLSLQIAATQPGDSAVYFCA |
| | | ETPDKLIFGTGTRLQVFPNIQNPEPAV |
| 17 | Vα only (without | MAGIRALFMYLWLQLDWVSRGESVGLHLPTLSVQEGDNSIIN |
| | the Constant) | CAYSNSASDYFIWYKQESGKGPQFIIDIRSNMDKRQGQRVTVL |
| | | LNKTVKHLSLQIAATQPGDSAVYFCAETPDKLIFGTGTRLQVFP |
| | | NIQNPEPAV |
| 18 | α chain with WT | MAGIRALFMYLWLQLDWVSRGESVGLHLPTLSVQEGDNSIIN |
| | signal peptide and | CAYSNSASDYFIWYKQESGKGPQFIIDIRSNMDKRQGQRVTVL |
| | constant Cα | LNKTVKHLSLQIAATQPGDSAVYFCAETPDKLIFGTGTRLQVFP |
| | | NIQNPEPAVNIQNPEPAVYQLKDPRSQDSTLCLFTDFDSQINVP |
| | | KTMESGTFITDKTVLDMKAMDSKSNGAIAWSNQTSFTCQDIF |
| | | KETNATYPSSDVPCDATLTEKSFETDMNLNFQNLLVIVLRILLLK |
| | | VAGFNLLMTLRLWSS |
| 19 | CDR1β | SEHNR |
| 20 | CDR2β | FQNEAQ |
| 21 | CDR3β | ASSGGGDSPLH |
| 22 | Vβ without signal | DTGVSQDPRHKITKRGQNVTFRCDPISEHNRLYWYRQTLGQG |
| | peptide (SignalP) | PEFLTYFQNEAQLEKSRLLSDRFSAERPKGSFSTLEIQRTEQGDS |
| | | AMYLCASSGGGDSPLHFGNGTRLTVTEDLRN |
| 23 | Vβ (without the | MGTSLLCWMALCLLGADHADTGVSQDPRHKITKRGQNVTFR |
| | Constant) | CDPISEHNRLYWYRQTLGQGPEFLTYFQNEAQLEKSRLLSDRFS |
| | | AERPKGSFSTLEIQRTEQGDSAMYLCASSGGGDSPLHFGNGTR |
| | | LTVTEDLRN |
| 24 | β chain with WT | MGTSLLCWMALCLLGADHADTGVSQDPRHKITKRGQNVTFR |
| | signal peptide and | CDPISEHNRLYWYRQTLGQGPEFLTYFQNEAQLEKSRLLSDRFS |
| | constant Cβ | AERPKGSFSTLEIQRTEQGDSAMYLCASSGGGDSPLHFGNGTR |
| | | LTVTEDLRNEDLRNVTPPKVSLFEPSKAEIANKQKATLVCLARGF |
| | | FPDHVELSWWVNGKEVHSGVSTDPQAYKESNYSYCLSSRLRV |
| | | SATFWHNPRNHFRCQVQFHGLSEEDKWPEGSPKPVTQNISAE |
| | | AWGRADCGITSASYQQGVLSATILYEILLGKATLYAVLVSTLVV |
| | | MAMVKRKNS |

In some aspects, 0013-TCR102 interacts with and/or is specific for a peptide from gene EGFR. In some aspects, the peptide is from a neoantigen of EGFR and has the amino acid change L858R (in which position 858 of the EGFR protein is mutated from Leu to Arg). In some aspects, 0013-TCR102 interacts with and/or is specific for the neoantigen in the context of HLA-A*11:01.

TABLE 3

| SEQ ID NO. | Description | 0013-TCR319 |
|---|---|---|
| 25 | CDR1α | YGATPY |
| 26 | CDR2α | YFSGDTLV |
| 27 | CDR3α | AVGGGGADGLT |
| 28 | Vα without signal | QSVTQPDIHITVSEGASLELRCNYSYGATPYLFWYVQSPGQGL |
| | peptide (SignalP) | QLLLKYFSGDTLVQGIKGFEAEFKRSQSSFNLRKPSVHWSDAAE |
| | | YFCAVGGGGADGLTFGKGTHLIIQPNIQNPEPAV |
| 29 | Vα only (without | MLLELIPLLGIHFVLRTARAQSVTQPDIHITVSEGASLELRCNYSY |
| | the Constant) | GATPYLFWYVQSPGQGLQLLLKYFSGDTLVQGIKGFEAEFKRS |
| | | QSSFNLRKPSVHWSDAAEYFCAVGGGGADGLTFGKGTHLIIQP |
| | | NIQNPEPAV |
| 30 | α chain with WT | MLLELIPLLGIHFVLRTARAQSVTQPDIHITVSEGASLELRCNYSY |
| | signal peptide and | GATPYLFWYVQSPGQGLQLLLKYFSGDTLVQGIKGFEAEFKRS |
| | constant Cα | QSSFNLRKPSVHWSDAAEYFCAVGGGGADGLTFGKGTHLIIQP |
| | | NIQNPEPAVNIQNPEPAVYQLKDPRSQDSTLCLFTDFDSQINVP |
| | | KTMESGTFITDKTVLDMKAMDSKSNGAIAWSNQTSFTCQDIF |
| | | KETNATYPSSDVPCDATLTEKSFETDMNLNFQNLLVIVLRILLLK |
| | | VAGFNLLMTLRLWSS |
| 31 | CDR1β | MDHEN |
| 32 | CDR2β | SYDVKM |
| 33 | CDR3β | ATLWYRQGADGYT |
| 34 | Vβ without signal | SRYLVKRTGEKVFLECVQDMDHENMFWYRQDPGLGLRLIYFS |
| | peptide (SignalP) | YDVKMKEKGDIPEGYSVSREKKERFSLILESASTNQTSMYLCATL |
| | | WYRQGADGYTFGSGTRLTVVEDLRN |
| 35 | Vβ (without the | MGIRLLCRVAFCFLAVGLVDVKVTQSSRYLVKRTGEKVFLECVQ |
| | Constant) | DMDHENMFWYRQDPGLGLRLIYFSYDVKMKEKGDIPEGYSVS |
| | | REKKERFSLILESASTNQTSMYLCATLWYRQGADGYTFGSGTRL |
| | | TVVEDLRN |
| 36 | β chain with WT | MGIRLLCRVAFCFLAVGLVDVKVTQSSRYLVKRTGEKVFLECVQ |
| | signal peptide and | DMDHENMFWYRQDPGLGLRLIYFSYDVKMKEKGDIPEGYSVS |
| | constant Cβ | REKKERFSLILESASTNQTSMYLCATLWYRQGADGYTFGSGTRL |
| | | TVVEDLRNEDLRNVTPPKVSLFEPSKAEIANKQKATLVCLARGF |
| | | FPDHVELSWWVNGKEVHSGVSTDPQAYKESNYSYCLSSRLRV |
| | | SATFWHNPRNHFRCQVQFHGLSEEDKWPEGSPKPVTQNISAE |
| | | AWGRADCGITSASYQQGVLSATILYEILLGKATLYAVLVSTLVV |
| | | MAMVKRKNS |

In some aspects, 0013-TCR319 interacts with and/or is specific for a peptide from gene EGFR. In some aspects, the peptide is from a neoantigen of EGFR and has the amino acid change L858R (in which position 858 of the EGFR protein is mutated from Leu to Arg). In some aspects, 0013-TCR319 interacts with and/or is specific for the neoantigen in the context of HLA-A*11:01.

The present disclosure provides a polynucleotide encoding an amino acid sequence that is at least 90%, 95%, 96%, 97%, 98%, 99%, or 100% identical to an amino acid sequence selected from the group consisting of SEQ ID NOs: 1-36 (the sequences provided in Tables 1-3).

In one aspect, the TCR used herein comprises a sequence selected from the TCR Cα or TCR Cβ provided in Tables 4 and 5.

TABLE 4
Amino acid sequences of TCR Cα regions.

| | | SEQ |
|---|---|---|
| | | ID |
| Description | Sequence | NO: |
| Cα (murine, | XIQNPEPAVYQLKDPRSQDSTLCLFTDFDSQINVPKTMESGTFITDKXVLDM | 37 |
| degenerate) | KAMDSKSNGAIAWSNQTSFTCQDIFKETNATYPSSDVPCDATLTEKSFETDM | |
| | NLNFQNLXVXXLRILLLKVAGENLLMTLRLWSS | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cα (murine, | NNNATCCAGAATCCCGAGCCTGCGGTGTACCAGCTGAAGGACCCCCGCTCTC | 38 |
| degenerate) | AGGATAGCACACTGTGCCTGTTCACCGACTTTGATAGCCAGATCAACGTGCC | |
| (exemplary | TAAAACAATGGAGTCCGGCACCTTCATCACCGACAAGNNNGTGCTGGATATG | |
| nucleotide | AAAGCGATGGACTCCAAGTCTAACGGCGCGATCGCGTGGTCCAATCAGACAT | |
| sequence) | CTTTCACCTGCCAGGATATCTTCAAGGAGACAAACGCGACCTATCCTTCCTC | |
| | TGACGTGCCATGTGATGCGACACTGACCGAGAAGAGCTTCGAGACAGACATG | |
| | AACCTGAATTTTCAGAATCTGNNNGTCNNNNNNCTGAGAATCCTGCTGCTGA | |
| | AGGTGGCGGGCTTTAATCTGCTGATGACACTGCGGCTGTGGAGTTCC | |
| | NNN at positions 1-3 make up a codon that encodes Asn, Asp, His, or | |
| | Tyr; | |
| | NNN at positions 142-144 make up a codon that encodes Thr or Cys; | |
| | NNN at positions 334-336 make up a codon that encodes Ser, Ala, Val, | |
| | Leu, Ile, Pro, Phe, Met, or Trp; | |
| | NNN at positions 340-342 make up a codon that encodes Met, Ala, Val, | |
| | Leu, Ile, Pro, Phe, or Trp; | |
| | NNN at positions 343-345 make up a codon that encodes Gly, Ala, Val, | |
| | Leu, Ile, Pro, Phe, Met, or Trp | |
| Cα (murine, | NIQNPEPAVYQLKDPRSQDSTLCLFTDFDSQINVPKTMESGTFITDKCVLDM | 39 |
| cysteine- | KAMDSKSNGAIAWSNQTSFTCQDIFKETNATYPSSDVPCDATLTEKSFETDM | |
| and LIV- | NLNFQNLLVIVLRILLLKVAGENLLMTLRLWSS | |
| substituted) | | |
| Cα (murine, | AACATCCAGAATCCCGAGCCTGCGGTGTACCAGCTGAAGGACCCCCGCTCTC | 40 |
| cysteine- | AGGATAGCACACTGTGCCTGTTCACCGACTTTGATAGCCAGATCAACGTGCC | |
| and LIV- | TAAAACAATGGAGTCCGGCACCTTCATCACCGACAAGTGCGTGCTGGATATG | |
| substituted) | AAAGCGATGGACTCCAAGTCTAACGGCGCGATCGCGTGGTCCAATCAGACAT | |
| (exemplary | CTTTCACCTGCCAGGATATCTTCAAGGAGACAAACGCGACCTATCCTTCCTC | |
| nucleotide | TGACGTGCCATGTGATGCGACACTGACCGAGAAGAGCTTCGAGACAGACATG | |
| sequence) | AACCTGAATTTTCAGAATCTGCTGGTCATCGTGCTGAGAATCCTGCTGCTGA | |
| | AGGTGGCGGGCTTTAATCTGCTGATGACACTGCGGCTGTGGAGTTCC | |
| Cα (murine, LIV | NIQNPEPAVYQLKDPRSQDSTLCLFTDFDSQINVPKTMESGTFITDKTVLDM | 41 |
| substituted) | KAMDSKSNGAIAWSNQTSFTCQDIFKETNATYPSSDVPCDATLTEKSFETDM | |
| | NLNFQNLLVIVLRILLLKVAGENLLMTLRLWSS | |
| Cα (murine, LIV | AACATCCAGAATCCCGAGCCTGCGGTGTACCAGCTGAAGGACCCCCGCTCTC | 42 |
| substituted) | AGGATAGCACACTGTGCCTGTTCACCGACTTTGATAGCCAGATCAACGTGCC | |
| (exemplary | TAAAACAATGGAGTCCGGCACCTTCATCACCGACAAGACCGTGCTGGATATG | |
| nucleotide | AAAGCGATGGACTCCAAGTCTAACGGCGCGATCGCGTGGTCCAATCAGACAT | |
| sequence) | CTTTCACCTGCCAGGATATCTTCAAGGAGACAAACGCGACCTATCCTTCCTC | |
| | TGACGTGCCATGTGATGCGACACTGACCGAGAAGAGCTTCGAGACAGACATG | |
| | AACCTGAATTTTCAGAATCTGCTGGTCATCGTGCTGAGAATCCTGCTGCTGA | |
| | AGGTGGCGGGCTTTAATCTGCTGATGACACTGCGGCTGTGGAGTTCC | |
| Cα (murine, | NIQNPEPAVYQLKDPRSQDSTLCLFTDFDSQINVPKTMESGTFITDKCVLDM | 43 |
| cysteine- | KAMDSKSNGAIAWSNQTSFTCQDIFKETNATYPSSDVPCDATLTEKSFETDM | |
| substituted) | NLNFQNLSVMGLRILLLKVAGENLLMTLRLWSS | |
| Cα (murine, | NIQNPEPAVYQLKDPRSQDSTLCLFTDFDSQINVPKTMESGTFITDKTVLDM | 44 |
| wild type) | KAMDSKSNGAIAWSNQTSFTCQDIFKETNATYPSSDVPCDATLTEKSFETDM | |
| | NLNFQNLSVMGLRILLLKVAGENLLMTLRLWSS | |
| Cα (human, | XIQNPDPAVYQLRDSKSSDKSVCLFTDFDSQTNVSQSKDSDVYITDKXVLDM | 45 |
| degenerate) | RSMDFKSNSAVAWSNKSDFACANAFNNSIIPEDTFFPSPESSCDVKLVEKSF | |
| | ETDTNLNFQNLXVXXFRILLLKVAGENLLMTLRLWSS | |
| | X at position 1 is Asn, Asp, His, or Tyr | |
| | X at position 48 is Thr or Cys; | |
| | X at position 116 is Ser, Ala, Val, Leu, Ile, Pro, Phe, Met, or Trp; | |
| | X at position 118 is Met, Ala, Val, Leu, Ile, Pro, Phe, or Trp; | |
| | X at position 119 is Gly, Ala, Val, Leu, Ile, Pro, Phe, Met, or Trp | |
| Cα (human, | XIQNPDPAVYQLRDSKSSDKSVCLFTDFDSQTNVSQSKDSDVYITDKCVLDM | 46 |
| cysteine- | RSMDFKSNSAVAWSNKSDFACANAFNNSIIPEDTFFPSPESSCDVKLVEKSF | |
| and LIV- | ETDTNLNFQNLLVIVFRILLLKVAGENLLMTLRLWSS | |
| substituted; | X at position 1 is Asn, Asp, His, or Tyr | |
| degenerate at | | |
| position 1) | | |
| Cα (human, LIV- | XIQNPDPAVYQLRDSKSSDKSVCLFTDFDSQTNVSQSKDSDVYITDKTVLDM | 47 |
| substituted; | RSMDFKSNSAVAWSNKSDFACANAFNNSIIPEDTFFPSPESSCDVKLVEKSF | |
| degenerate | ETDTNLNFQNLLVIVFRILLLKVAGENLLMTLRLWSS | |
| at position 1) | X at position 1 is Asn, Asp, His, or Tyr | |
| Cα (human, | XIQNPDPAVYQLRDSKSSDKSVCLFTDFDSQTNVSQSKDSDVYITDKCVLDM | 48 |
| cysteine- | RSMDFKSNSAVAWSNKSDFACANAFNNSIIPEDTFFPSPESSCDVKLVEKSF | |
| substituted; | ETDTNLNFQNLSVIGFRILLLKVAGENLLMTLRLWSS | |
| degenerate | X at position 1 is Asn, Asp, His, or Tyr | |
| at position 1) | | |
| Cα (human, | XIQNPDPAVYQLRDSKSSDKSVCLFTDFDSQTNVSQSKDSDVYITDKTVLDM | 49 |
| wild type; | RSMDFKSNSAVAWSNKSDFACANAFNNSIIPEDTFFPSPESSCDVKLVEKSF | |
| degenerate at | ETDTNLNFQNLSVIGFRILLLKVAGENLLMTLRLWSS | |
| position 1) | X at position 1 is Asn, Asp, His, or Tyr | |

TABLE 5
Amino acid sequences of TCR Cβ regions.

| | | SEQ |
|---|---|---|
| | | ID |
| Description | Sequence | NO: |
| Cβ (murine, | EDLRNVTPPKVSLFEPSKAEIANKQKATLVCLARGFFPDHVELSWWVNGKEV | 50 |
| degenerate) | HSGVXTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| | X at position 57 is Ser or Cys | |
| Cβ (murine, | GAGGACCTGAGGAACGTGACCCCACCTAAAGTGAGCCTGTTCGAGCCATCCA | 51 |
| degenerate) | AGGCGGAGATCGCGAATAAGCAGAAAGCGACCCTGGTGTGCCTGGCGAGGGG | |
| (exemplary | CTTCTTTCCCGATCACGTGGAGCTGTCCTGGTGGGTGAACGGCAAAGAGGTG | |
| nucleotide | CACTCTGGCGTGNNNACAGACCCTCAGGCGTACAAGGAGAGCAATTACTCCT | |
| sequence) | ATTGTCTGTCTAGCAGACTGAGGGTGAGCGCGACCTTTTGGCACAACCCCCG | |
| | GAATCACTTCCGCTGCCAGGTGCAGTTTCACGGCCTGTCCGAGGAGGATAAA | |
| | TGGCCTGAGGGCTCTCCAAAGCCCGTGACACAGAATATCAGCGCGGAGGCGT | |
| | GGGGAAGAGCGGACTGTGGCATTACAAGCGCGTCCTATCAGCAGGGCGTGCT | |
| | GTCCGCGACCATCCTGTACGAGATTCTGCTGGGCAAGGCGACACTGTATGCG | |
| | GTGCTGGTGTCCACCCTGGTGGTCATGGCGATGGTGAAGAGGAAAAACTCT | |
| | NNN at positions 169-171 make up a codon that encodes | |
| | Ser or Cys | |
| Cβ (murine, | EDLRNVTPPKVSLFEPSKAEIANKQKATLVCLARGFFPDHVELSWWVNGKEV | 52 |
| cysteine- | HSGVCTDPQAYKESNYSYCLSSRLRVSATFWHNPRNHFRCQVQFHGLSEEDK | |
| substituted) | WPEGSPKPVTQNISAEAWGRADCGITSASYQQGVLSATILYEILLGKATLYA | |
| | VLVSTLVVMAMVKRKNS | |
| Cβ (murine, | GAGGACCTGAGGAACGTGACCCCACCTAAAGTGAGCCTGTTCGAGCCATCCA | 53 |
| cysteine- | AGGCGGAGATCGCGAATAAGCAGAAAGCGACCCTGGTGTGCCTGGCGAGGGG | |
| substituted) | CTTCTTTCCCGATCACGTGGAGCTGTCCTGGTGGGTGAACGGCAAAGAGGTG | |
| (exemplary | CACTCTGGCGTGTGCACAGACCCTCAGGCGTACAAGGAGAGCAATTACTCCT | |
| nucleotide | ATTGTCTGTCTAGCAGACTGAGGGTGAGCGCGACCTTTTGGCACAACCCCCG | |
| sequence) | GAATCACTTCCGCTGCCAGGTGCAGTTTCACGGCCTGTCCGAGGAGGATAAA | |
| | TGGCCTGAGGGCTCTCCAAAGCCCGTGACACAGAATATCAGCGCGGAGGCGT | |
| | GGGGAAGAGCGGACTGTGGCATTACAAGCGCGTCCTATCAGCAGGGCGTGCT | |
| | GTCCGCGACCATCCTGTACGAGATTCTGCTGGGCAAGGCGACACTGTATGCG | |
| | GTGCTGGTGTCCACCCTGGTGGTCATGGCGATGGTGAAGAGGAAAAACTCT | |
| Cβ (murine, | EDLRNVTPPKVSLFEPSKAEIANKQKATLVCLARGFFPDHVELSWWVNGKEV | 54 |
| wild type) | HSGVSTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| Cβ (human, | EDLKNVFPPEVAVFEPSEAEISHTQKATLVCLATGFYPDHVELSWWVNGKEV | 55 |
| degenerate) | HSGVXTDPQPLKEQPALNDSRYCLSSRLRVSATFWQNPRNHFRCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |
| | X at position 57 is Ser or Cys | |
| Cβ (human, | EDLKNVEPPEVAVFEPSEAEISHTQKATLVCLATGFYPDHVELSWWVNGKEV | 56 |
| cysteine- | HSGVCTDPQPLKEQPALNDSRYCLSSRLRVSATFWQNPRNHERCQVQFYGLS | |
| substituted) | ENDEWTQDRAKPVTQIVSAEAWGRADCGFTSESYQQGVLSATILYEILLGKA | |
| | TLYAVLVSALVLMAMVKRKDSRG | |
| Cβ (human, | EDLKNVFPPEVAVFEPSEAEISHTQKATLVCLATGFYPDHVELSWWVNGKEV | 57 |
| wild type) | HSGVSTDPQPLKEQPALNDSRYCLSSRLRVSATFWQNPRNHFRCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |

Non-limiting examples of HLA sequences and neoantigen peptide sequences are provided in Table 6 below. All the sequences are human.

TABLE 6

| SEQ ID | | | |
|---|---|---|---|
| NO: | Description | Sequence type | Sequence |
| 58 | HLA-A*03:01 | Restricting | MAVMAPRTLLLLLSGALALTQTWAGSHSMRYF |
| | | HLA | FTSVSRPGRGEPRFIAVGYVDDTQFVRFDSDAAS |
| | | | QRMEPRAPWIEQEGPEYWDQETRNVKAQSQT |
| | | | DRVDLGTLRGYYNQSEAGSHTIQIMYGCDVGSD |
| | | | GRFLRGYRQDAYDGKDYIALNEDLRSWTAADM |
| | | | AAQITKRKWEAAHEAEQLRAYLDGTCVEWLRRY |
| | | | LENGKETLQRTDPPKTHMTHHPISDHEATLRCW |
| | | | ALGFYPAEITLTWQRDGEDQTQDTELVETRPAG |
| | | | DGTFQKWAAVVVPSGEEQRYTCHVQHEGLPKP |
| | | | LTLRWELSSQPTIPIVGIIAGLVLLGAVITGAVVAA |
| | | | VMWRRKSSDRKGGSYTQAASSDSAQGSDVSLT |
| | | | ACKV |
| 59 | HLA-B*35:02 | Restricting | MRVTAPRTVLLLLWGAVALTETWAGSHSMRYF |
| | | HLA | YTAMSRPGRGEPRFIAVGYVDDTQFVRFDSDAA |
| | | | SPRTEPRAPWIEQEGPEYWDRNTQIFKTNTQTY |
| | | | RESLRNLRGYYNQSEAGSHIIQRMYGCDLGPDG |
| | | | RFLRGHNQYAYDGKDYIALNEDLSSWTAADTAA |
| | | | QITQRKWEAARVAEQLRAYLEGLCVEWLRRYLE |
| | | | NGKETLQRADPPKTHVTHHPVSDHEATLRCWA |
| | | | LGFYPAEITLTWQRDGEDQTQDTELVETRPAGD |
| | | | RTFQKWAAVVVPSGEEQRYTCHVQHEGLPKPL |
| | | | TLRWEPSSQSTIPIVGIVAGLAVLAVVVIGAVVAT |
| | | | VMCRRKSSGGKGGSYSQAASSDSAQGSDVSLT |
| | | | A |
| 60 | HLA- | Restricting | MRPEDRMFHIRAVILRALSLAFLLSLRGAGAIKA |
| | DPA1*01:03 | HLA | DHVSTYAAFVQTHRPTGEFMFEFDEDEMFYVD |
| | | | LDKKETVWHLEEFGQAFSFEAQGGLANIAILNN |
| | | | NLNTLIQRSNHTQATNDPPEVTVFPKEPVELGQ |
| | | | PNTLICHIDKFFPPVLNVTWLCNGELVTEGVAES |
| | | | LFLPRTDYSFHKFHYLTFVPSAEDFYDCRVEHWG |
| | | | LDQPLLKHWEAQEPIQMPETTETVLCALGLVLGL |
| | | | VGIIVGTVLIIKSLRSGHDPRAQGTL |
| 61 | HLA- | Restricting | MRPEDRMFHIRAVILRALSLAFLLSLRGAGAIKA |
| | DPA1*03:01 | HLA | DHVSTYAMFVQTHRPTGEFMFEFDEDEMFYVD |
| | | | LDKKETVWHLEEFGQAFSFEAQGGLANIAISNN |
| | | | NLNTLIQRSNHTQATNDPPEVTVFPKEPVELGQ |
| | | | PNTLICHIDKFFPPVLNVTWLCNGELVTEGVAES |
| | | | LFLPRTDYSFHKFHYLTFVPSAEDFYDCRVEHWG |
| | | | LDQPLLKHWEAQEPIQMPETTETVLCALGLVLGL |
| | | | VGIIVGTVLIIKSLRSGHDPRAQGTL |
| 62 | HLA- | Restricting | MMVLQVSAAPRTVALTALLMVLLTSVVQGRAT |
| | DPB1*104:01 | HLA | PENYVYQLRQECYAFNGTQRFLERYIYNREEFVR |
| | | | FDSDVGEFRAVTELGRPDEDYWNSQKDLLEEKR |
| | | | AVPDRVCRHNYELDEAVTLQRRVQPKVNVSPSK |
| | | | KGPLQHHNLLVCHVTDFYPGSIQVRWFLNGQEE |
| | | | TAGVVSTNLIRNGDWTFQILVMLEMTPQQGDV |
| | | | YICQVEHTSLDSPVTVEWKAQSDSARSKTLTGA |
| | | | GGFMLGLIICGVGIFMHRRSKKVQRGSA |
| 63 | HLA- | Restricting | MAISGVPVLGFFIIAVLMSAQESWAIKEEHVIIQA |
| | DRA*01:01 | HLA | EFYLNPDQSGEFMFDFDGDEIFHVDMAKKETV |
| | | | WRLEEFGRFASFEAQGALANIAVDKANLEIMTK |
| | | | RSNYTPITNVPPEVTVLTNSPVELREPNVLICFIDK |
| | | | FTPPVVNVTWLRNGKPVTTGVSETVFLPREDHL |
| | | | FRKFHYLPFLPSTEDVYDCRVEHWGLDEPLLKH |
| | | | WEFDAPSPLPETTENVVCALGLTVGLVGIIIGTIFII |
| | | | KGVRKSNAAERRGPL |
| 64 | HLA- | Restricting | MVCLKLPGGSCMTALTVTLMVLSSPLALAGDTR |
| | DRB1*01:01 | HLA | PRFLWQLKFECHFFNGTERVRLLERCIYNQEESV |
| | | | RFDSDVGEYRAVTELGRPDAEYWNSQKDLLEQR |
| | | | RAAVDTYCRHNYGVGESFTVQRRVEPKVTVYPS |
| | | | KTQPLQHHNLLVCSVSGFYPGSIEVRWFRNGQE |
| | | | EKAGVVSTGLIQNGDWTFQTLVMLETVPRSGEV |
| | | | YTCQVEHPSVTSPLTVEWRARSESAQSKMLSGV |
| | | | GGFVLGLLFLGAGLFIYFRNQKGHSGLQPTGFLS |
| 65 | HLA- | Restricting | MVCLRLPGGSCMAVLTVTLMVLSSPLALAGDTR |
| | DRB1*11:01 | HLA | PRFLEYSTSECHFFNGTERVRFLDRYFYNQEEYVR |
| | | | FDSDVGEFRAVTELGRPDEEYWNSQKDFLEDRR |
| | | | AAVDTYCRHNYGVGESFTVQRRVHPKVTVYPSK |
| | | | TQPLQHHNLLVCSVSGFYPGSIEVRWFRNGQEE |
| | | | KTGVVSTGLIHNGDWTFQTLVMLETVPRSGEVY |
| | | | TCQVEHPSVTSPLTVEWRARSESAQSKMLSGVG |
| | | | GFVLGLLFLGAGLFIYFRNQKGHSGLQPRGFLS |
| 66 | HLA- | Restricting | MVCLKLPGGSCMAALTVTLTVLSSPLALAGDTQ |
| | DRB4*01:03 | HLA | PRFLEQAKCECHFLNGTERVWNLIRYIYNQEEYA |
| | | | RYNSDLGEYQAVTELGRPDAEYWNSQKDLLERR |
| | | | RAEVDTYCRYNYGVVESFTVQRRVQPKVTVYPS |
| | | | KTQPLQHHNLLVCSVNGFYPGSIEVRWFRNGQE |
| | | | EKAGVVSTGLIQNGDWTFQTLVMLETVPRSGEV |
| | | | YTCQVEHPSMMSPLTVQWSARSESAQSKMLSG |
| | | | VGGFVLGLLFLGTGLFIYFRNQKGHSGLQPTGLL |
| | | | S |
| 67 | 2599_TWNK_ | Mutation/ | RRRYRKETLQALYMPVLPVTATEIR |
| | p.D49Y | Peptide | |
| 68 | 2599_SOX6_ | Mutation/ | TSREKEEGSDQHEASHLPLHPIMHN |
| | p.V37E | Peptide | |
| 69 | 2599_NAA40_ | Mutation/ | VLYCYEVQLESKLRRKGLGKFLIQI |
| | p.V146L | Peptide | |
| 70 | 2599_ATG2A_ | Mutation/ | TTNLGGPECCLRISLMPLRLNVDQD |
| | p.V1580I | Peptide | |
| 71 | 2599_SLCO1B3_ | Mutation/ | SHMWIYVFMGNMRRGIGETPIVPLG |
| | p.L180R | Peptide | |
| 72 | 2599_ERGIC2_ | Mutation/ | SQSPNACRIHGHPYVNKVAGNFHIT |
| | p.L176P | Peptide | |
| 73 | 2599_CSAD_ | Mutation/ | PSARRSYLCTLPLALLSREILMADS |
| | p.P88L | Peptide | |
| 74 | 2599_CSAD_ | Mutation/ | SSFLLSYLCTLPLALLSREILMADS |
| | p.P19L | Peptide | |
| 75 | 2599_NAV3_ | Mutation/ | EEKAHSEQIHKLWRELVASQEKVAT |
| | p.R1575W | Peptide | |
| 76 | 2599_ABHD13_ | Mutation/ | LIALASWSWALCCISLLPLIVTFHL |
| | p.R27C | Peptide | |
| 77 | 2599_NBEA_ | Mutation/ | VNDVDLPPWAKKTEDFVRINRMALE |
| | p.P2474T | Peptide | |
| 78 | 2599_THSD1_ | Mutation/ | HVGSRGGPSERSRARNAHFRRTASF |
| | p.H644R | Peptide | |
| 79 | 2599_GPC6_ | Mutation/ | FENLVEETSHFVCTTFVSRHKKFDE |
| | p.R95C | Peptide | |
| 80 | 2599_CERS3_ | Mutation/ | EMSFYWSLLFRLVFDVKRKDFLAHI |
| | p.G197V | Peptide | |
| 81 | 2599_GLDN_ | Mutation/ | GEKGDKGDVSNDMLLAGAKGDQGPP |
| | p.V226M | Peptide | |
| 82 | 2599_FAM219B_ | Mutation/ | DEDLDLIPPKPMVSSTCSCCWCCLG |
| | p.A178V | Peptide | |
| 83 | 2599_BAIAP3_ | Mutation/ | ELSTPAATILCLLGAQSNLSPLQLA |
| | p.H425L | Peptide | |
| 84 | 2599_CLEC19A_ | Mutation/ | LFWMEFKGHCYRLFPLNKTWAEADL |
| | p.F54L | Peptide | |
| 85 | 2599_ZPBP2_ | Mutation/ | RSCIGRYNDVFFGVLKKILDSLISD |
| | p.R165G | Peptide | |
| 86 | 2599_ASB16_ | Mutation/ | APLAIATARGYTGCARHLIRQGAEL |
| | p.D125G | Peptide | |
| 87 | 2599_ARHGAP27_ | Mutation/ | SESSRVDFGSSEHLGSWQEKEEDAR |
| | p.R635H | Peptide | |
| 88 | 2599_TACO1_ | Mutation/ | RHIKGPKDVERSPIFSKLCLNIRLA |
| | p.R79P | Peptide | |
| 89 | 2599_TP53_ | Mutation/ | SGNLLGRNSFEVCVCACPGRDRRTE |
| | p.R273C | Peptide | |
| 90 | 2599_PLEKHG2_ | Mutation/ | GSWSSAPTSRASLPPPQPQPPPPPA |
| | p.S1318L | Peptide | |
| 91 | 2599_ZNF229_ | Mutation/ | HQKTHTGERPYQWDKCGKGFSHNSY |
| | p.C491W | Peptide | |
| 92 | 2599_TRIM33_ | Mutation/ | HSQHYQIPDDFVPDVRLIFKNCERF |
| | p.A1026P | Peptide | |
| 93 | 2599_MLLT11_ | Mutation/ | LFWRMPIPELDLLELEGLGLSDTAT |
| | p.S25L | Peptide | |
| 94 | 2599_SMCP_ | Mutation/ | QNQCCQSKGNQCYPPKQNQCCQPKG |
| | p.C35Y | Peptide | |
| 95 | 2599_LRRN2_ | Mutation/ | ITNNPRLSFIHPHAFHHLPQMETLM |
| | p.R329H | Peptide | |
| 96 | 2599_HHIPL2_ | Mutation/ | RGLQESHGRDGTHFCHLLDLPDKDY |
| | p.R167H | Peptide | |
| 97 | 2599_RYR2_ | Mutation/ | VLNYFQPFLGRIKIMGSAKRIERVY |
| | p.E4156K | Peptide | |
| 98 | 2599_OR2M4_ | Mutation/ | TSAFERLLVICCAVMLIFPVSVIIL |
| | p.V204A | Peptide | |
| 99 | 2599_OR2T3_ | Mutation/ | ILHLIHRMNSAASHRKALATCSSHM |
| | p.G238S | Peptide | |
| 100 | 2599_LEPROT_ | Mutation/ | LAGNAVIFLTIQRFFLIFGRGDDFS |
| | p.G124R | Peptide | |
| 101 | 2599_FPGT_ | Mutation/ | LTKAALPAHSFVGSLSLKMNRCLKY |
| | p.C467G | Peptide | |
| 102 | 2599_NAPB_ | Mutation/ | EMFPAFTDSRECQLLKKLLEAHEEQ |
| | p.K246Q | Peptide | |
| 103 | 2599_ITCH_ | Mutation/ | QRSQLQGAMQQFKQRFIYGNQDLFA |
| | p.N415K | Peptide | |
| 104 | 2599_LONRF2_ | Mutation/ | CPRCRRLLHKPVMLPCGLTVCKRCV |
| | p.T155M | Peptide | |
| 105 | 2599_IL1RL1_ | Mutation/ | TRNDGKLYDAYVFYPRNYKSSTDGA |
| | p.V382F | Peptide | |
| 106 | 2599_AMER3_ | Mutation/ | ASAPECRCSLLACEGLLCGQPEVGA |
| | p.R839C | Peptide | |
| 107 | 2599_LCT_ | Mutation/ | IEGAWRADGKGLIIWDTFSHTPLRV |
| | p.S1404I | Peptide | |
| 108 | 2599_DPP4_ | Mutation/ | NIIVASFDGRGSVYQGDKIMHAINR |
| | p.G584V | Peptide | |
| 109 | 2599_TLK1_ | Mutation/ | EDIERQRKLLAKCKPPTANNSQAPS |
| | p.R370C | Peptide | |
| 110 | 2599_FSIP2_ | Mutation/ | QMFSVSEISTVAHEITDSVLNILHK |
| | p.Q1257H | Peptide | |
| 111 | 2599_CFAP44_ | Mutation/ | KQLIREKREMTKNIHKMEETVRQLM |
| | p.T1734N | Peptide | |
| 112 | 2599_CLSTN2_ | Mutation/ | AGLLVDSSEMIFNFDGRQGAKVPDG |
| | p.K357N | Peptide | |
| 113 | 2599_FGD5_ | Mutation/ | SAQRWIEAMEDATVL |
| | p.S1503T | Peptide | |
| 114 | 2599_LRRC2_ | Mutation/ | MESERDRQHFDKVVMKAYIEDLKER |
| | p.E343V | Peptide | |
| 115 | 2599_PDZRN3_ | Mutation/ | AQLELQMTALRYRKKFTEYSARLDS |
| | p.Q217R | Peptide | |
| 116 | 2599_CRYBG3_ | Mutation/ | FQEHFGIYTGKIFIDFPTAAQFDNL |
| | p.S1137F | Peptide | |
| 117 | 2599_FAT4_ | Mutation/ | PAIVGSCATVLAFLVLSLILCNQCR |
| | p.L4520F | Peptide | |
| 118 | 2599_CCDC149_ | Mutation/ | KGIPEGGGMRSTMKT |
| | p.V527M | Peptide | |
| 119 | 2599_EVC_ | Mutation/ | LSRTFLRVNAFPGVLACESVDVDLC |
| | p.E200G | Peptide | |
| 120 | 2599_NPFFR2_ | Mutation/ | VMEELKETTNSSKI |
| | p.E521K | Peptide | |
| 121 | 2599_KLHL3_ | Mutation/ | DQWTSIASMQERQSTLGAAVLNDLL |
| | p.R352Q | Peptide | |
| 122 | 2599_PCDHA9_ | Mutation/ | QLTIKTLSVPVKKDAQLGTVIALIS |
| | p.E361K | Peptide | |
| 123 | 2599_PCDHA9_ | Mutation/ | ESVSAYELVVTAQDRGSPSLWATAR |
| | p.R428Q | Peptide | |
| 124 | 2599_GRIA1_ | Mutation/ | VNLAVLKLSEQGILDKLKSKWWYDK |
| | p.V782I | Peptide | |
| 125 | 2599_RXFP3_ | Mutation/ | GVVVYSGGRYDLMPSSSAY |
| | p.L463M | Peptide | |
| 126 | 2599_GDNF_ | Mutation/ | AANMPEDYPDQFHDVMDFIQATIKR |
| | p.D53H | Peptide | |
| 127 | 2599_GDNF_ | Mutation/ | DSNMPEDYPDQFHDVMDFIQATIKR |
| | p.D79H | Peptide | |
| 128 | 2599_STX11_ | Mutation/ | QHGPHSAVARISWAQYNALTLTFQR |
| | p.R129W | Peptide | |
| 129 | 2599_OR2B3_ | Mutation/ | YFFLTNLSILDLYYTTTTVPHMLVN |
| | p.C72Y | Peptide | |
| 130 | 2599_THSD7A_ | Mutation/ | GSSRTVWCQRSDCINVTGGCLVMSQ |
| | p.G1498C | Peptide | |
| 131 | 2599_FLNC_ | Mutation/ | KHTIIISWGGVNMPKSPFRVNVGEG |
| | p.V749M | Peptide | |
| 132 | 2599_SVOPL_ | Mutation/ | ERDLVCGSKSDSGVVVTGGDSGESQ |
| | p.A319G | Peptide | |
| 133 | 2599_SLC37A3_ | Mutation/ | AKETGSHIEGVTSARETERTMSATS |
| | p.G396S | Peptide | |
| 134 | 2599_DNAH11_ | Mutation/ | YALRNFVEEKLGVKYVERTRLDLVK |
| | p.A3906V | Peptide | |
| 135 | 2599_CRHR2_ | Mutation/ | ILMTKLRASTTSKTIQYRKAVKATL |
| | p.E301K | Peptide | |
| 136 | 2599_TECPR1_ | Mutation/ | PSPQAIWSITCKEDIFVSEPSPDLE |
| | p.G738E | Peptide | |
| 137 | 2599_DCAF13_ | Mutation/ | SRNPDNYVRETKFDLQRVPRNYDPA |
| | p.L171F | Peptide | |
| 138 | 2599_TMEM2_ | Mutation/ | LGVLEQFIPLQLHEYGCPRATTVRR |
| | p.D1358H | Peptide | |
| 139 | 2599_MAGEC1_ | Mutation/ | PHYFPQSPPQGEGSLSPHYFPQSPQ |
| | p.D573G | Peptide | |
| 140 | 2599_PHEX_ | Mutation/ | LGYIKKVIDTRLFPHLKDISPSENV |
| | p.Y327F | Peptide | |
| 141 | 2599_PCDH19_ | Mutation/ | NPMPIRSKSPEHMRNIIALSIEATA |
| | p.V987M | Peptide | |
| 142 | 2599_ERGIC2_ | Mutation/ | RIHGHPYVNK |
| | p.L176P* | Peptide | |
| 143 | 8434_ANXA9_ | Mutation/ | ALLGLASVIKNTSLYFADKLHQALQ |
| | p.P272S | Peptide | |
| 144 | 8434_ANXA9_ | Mutation/ | GSARPSFGDQEHIAVLC |
| | p.T268I | Peptide | |
| 145 | 8434_DCHS2_ | Mutation/ | LIPGNVSSLFTIESTTGIIYLTLPL |
| | p.D822-1 | Peptide | |
| 146 | 8434_PRRC2C_ | Mutation/ | LASAPLPPSTLASVSASASVSASVP |
| | p.P1763S | Peptide | |
| 147 | 8434_CRB1_ | Mutation/ | AEKEPEFLNISIHDSRLFFQLQSGN |
| | p.Q980H | Peptide | |
| 148 | 8434_TRIM67_ | Mutation/ | PEHEMENYSMYCMSCRTPVCYLCLE |
| | p.V316M | Peptide | |
| 149 | 8434_RYR2_ | Mutation/ | HKNPVPQCPPRLRVQFLSHVLWSRM |
| | p.H1587R | Peptide | |
| 150 | 8434_SPOCD1_ | Mutation/ | SCRLVQALPTVICSAGCIPSNIVWD |
| | p.R902C | Peptide | |
| 151 | 8434_ARHGEF16_ | Mutation/ | DKDPGGMLRRNLWNQSYRAAMKGLG |
| | p.R150W | Peptide | |
| 152 | 8434_CSMD2_ | Mutation/ | HCVWLILARPESHIHLAFNDIDVEP |
| | p.R655H | Peptide | |
| 153 | 8434_ZCCHC11_ | Mutation/ | SLPPPSPAHLAAFSVAVIELAKEHG |
| | p.L355F | Peptide | |
| 154 | 8434_SGMS1_ | Mutation/ | FCIVGTLYLYRCLTMYVTTLPVPGM |
| | p.I228L | Peptide | |
| 155 | 8434_DYNC2H1_ | Mutation/ | HKEWIVIGQVDMAALVEKHLFTVHD |
| | p.E869A | Peptide | |
| 156 | 8434_CWF19L2_ | Mutation/ | TITQIPKKSGVEYEDQQEVILVRTD |
| | p.N540Y | Peptide | |
| 157 | 8434_ESAM_ | Mutation/ | VSLVYSMPSRNLYLRLEGLQEKDSG |
| | p.S110Y | Peptide | |
| 158 | 8434_METTL15_ | Mutation/ | WLESGIPNLGVWAKRIHTTAEKYRE |
| | p.P30A | Peptide | |
| 159 | 8434_LRP4_ | Mutation/ | ETVIGRGLKTTDRLAVDWVARNLYW |
| | p.G1432R | Peptide | |
| 160 | 8434_FOLH1_ | Mutation/ | VQAAAETLSEVAQ |
| | p.L720Q | Peptide | |
| 161 | 8434_UTP20_ | Mutation/ | VRGYQVHVLTFTIHMLLQGLTNKLQ |
| | p.V1905I | Peptide | |
| 162 | 8434_KRAS_ | Mutation/ | ETCLLDILDTAGHEEYSAMRDQYMR |
| | p.Q61H | Peptide | |
| 163 | 8434_PARP4_ | Mutation/ | FSDSLSTSIKYSQPGETDGTRLLLI |
| | p.H490Q | Peptide | |
| 164 | 8434_NPAS3_ | Mutation/ | KVERYVESESDLLLQNCESLTSDSA |
| | p.R568L | Peptide | |
| 165 | 8434_BAZ1A_ | Mutation/ | TKDLTEALDEDANPTKSALSAVASL |
| | p.D514N | Peptide | |
| 166 | 8434_FANCM_ | Mutation/ | LNSKSESLPVSDNTAISETPLVSQF |
| | p.K1138N | Peptide | |
| 167 | 8434_ASB2_ | Mutation/ | PPAPQPSSRFNDVPAADKEPSVVQF |
| | p.A528V | Peptide | |
| 168 | 8434_RYR3_ | Mutation/ | VYLYTVVAFNFFCKFYNKSEDDDEP |
| | p.R4693C | Peptide | |
| 169 | 8434_C15orf41_ | Mutation/ | SIFSQEYQKHIKTTHAKHHTSEAIE |
| | p.R54T | Peptide | |
| 170 | 8434_FEM1B_ | Mutation/ | NRVKNISDADVHTAMDNYECNLYTF |
| | p.N435T | Peptide | |
| 171 | 8434_PRR35_ | Mutation/ | PKASPSLTRFCSQSSLPTGSSVMLW |
| | p.R359Q | Peptide | |
| 172 | 8434_BEAN1_ | Mutation/ | SFKRPCPLARYNHTSYFYPTFSESS |
| | p.R14H | Peptide | |
| 173 | 8434_CES2_ | Mutation/ | QFWKKALPQKIQKLEEPEERHTEL |
| | p.E612K | Peptide | |
| 174 | 8434_DHX38_ | Mutation/ | DILFSKTPQEDYMEAAVKQSLQVHL |
| | p.V724M | Peptide | |
| 175 | 8434_STUB1_ | Mutation/ | ERRIHQESELHSCLSRLIAAERERE |
| | p.Y164C | Peptide | |
| 176 | 8434_GRIN2A | Mutation/ | TCVRNTVPCRKFIKINNSTNEGMNV |
| | p. V440I | Peptide | |
| 177 | 8434_HS3ST3B1_ | Mutation/ | GTRALLEFLRVHSDVRAVGAEPHFF |
| | p.P161S | Peptide | |
| 178 | 8434_TMEM132E_ | Mutation/ | VFLINCIVFVLRCRHKRIPPEGQTS |
| | p.Y916C | Peptide | |
| 179 | 8434_TP53_ | Mutation/ | YMCNSSCMGGMNQRPILTIITLEDS |
| | p.R248Q | Peptide | |
| 180 | 8434_SALL3_ | Mutation/ | AATDPAKPLLSYEGSCPPSPPSVIS |
| | p.A826E | Peptide | |
| 181 | 8434_ZNF443_ | Mutation/ | CKECGKSFSSLGILQRHMAVQRGDG |
| | .pN185I | Peptide | |
| 182 | 8434_CACNA1A_ | Mutation/ | NGYYPAHGLARPHGPGSRKGLHEPY |
| | p.R2486H | Peptide | |
| 183 | 8434_BABAM1_ | Mutation/ | ALELHNCMAKLLSHPLQRPCQSHAS |
| | p.A300S | Peptide | |
| 184 | 8434_NPHS1_ | Mutation/ | ESRRVHLGSVEKYGSTFSRELVLVT |
| | p.S505Y | Peptide | |
| 185 | 8434_PNMAL2_ | Mutation/ | QPDLPPQAKKAGCGLFGGWSEHRED |
| | p.R432C | Peptide | |
| 186 | 8434_SBK2_ | Mutation/ | FLYEFCVGLSLGTHSAIVTAYGIGI |
| | p.A115T | Peptide | |
| 187 | 8434_ZNF865_ | Mutation/ | KSFNRRESLKRHAKTHSADLLRLPC |
| | p.V397A | Peptide | |
| 188 | 8434_REV1_ | Mutation/ | KNPLLHLKAAVKGKKRNKKKKTIGS |
| | p.E1069G | Peptide | |
| 189 | 8434_OLA1_ | Mutation/ | QGRNYIVEDGDINFFKFNTPQQPKK |
| | p.I225N | Peptide | |
| 190 | 8434_ABCA12_ | Mutation/ | SQTTLEEVFINFSKDQKSYETADTS |
| | p.A2248S | Peptide | |
| 191 | 8434_EPAS1_ | Mutation/ | RFPPQCYATQYQNYSLSSAHKVSGM |
| | p.D810N | Peptide | |
| 192 | 8434_DOPEY2_ | Mutation/ | LYLPLIQERLTDILRVGQTSIVAAQ |
| | p.N2059I | Peptide | |
| 193 | 8434_TXNRD2_ | Mutation/ | ACLPTTVGHAGKKQRRD |
| | p.N334K | Peptide | |
| 194 | 8434_CELSR1_ | Mutation/ | PSEDLQEQIYLNWTLLTTISTQRVL |
| | p.R1288W | Peptide | |
| 195 | 8434_MYLK_ | Mutation/ | CASDIRSSSLTLTWYGSSYDGGSAV |
| | p.S1175T | Peptide | |
| 196 | 8434_KCNAB1_ | Mutation/ | ISEENTKLRRQSSFSVAGKDKSPKK |
| | p.G25S | Peptide | |
| 197 | 8434_SI_ | Mutation/ | MARKKFCGLEISLIVLFVI |
| | p.S7C | Peptide | |
| 198 | 8434_RAB5A_ | Mutation/ | GNKICQFKLVLLEESAVGKSSLVLR |
| | p.G27E | Peptide | |
| 199 | 8434_CSPG5_ | Mutation/ | SAALVLLLLFMMMVFFAKKLYLLKT |
| | p.T440M | Peptide | |
| 200 | 8434_BSN_ | Mutation/ | RPLKSAEEAYEELMRKAELLQRQQG |
| | p.M1191L | Peptide | |
| 201 | 8434_ITIH3_ | Mutation/ | SQKDYRKDASIGMKVVCWFVHNNGE |
| | p.T862M | Peptide | |
| 202 | 8434_OR5H14_ | Mutation/ | LYPAIMTNGLCIQLLILSYVGGLLH |
| | p.R143Q | Peptide | |
| 203 | 8434_TRPC3_ | Mutation/ | NSKSRLNLFTQSISRVFESHSENSI |
| | p.N829I | Peptide | |
| 204 | 8434_FBXW7_ | Mutation/ | VETGNCIHTLTGQQSLTSGMELKDN |
| | p.H580Q | Peptide | |
| 205 | 8434_NSD2_ | Mutation/ | PPPEPGKPKGKRWRRRGWRRVTEGK |
| | p.R1353W | Peptide | |
| 206 | 8434_MYO10_ | Mutation/ | SRFDTDDELSYRHDSVYSCVTLPYF |
| | p.R1166H | Peptide | |
| 207 | 8434_ROS1_ | Mutation/ | ERMHFIHRDLAASNCLVSVKDYTSP |
| | p.R2083S | Peptide | |
| 208 | 8434_MCM9_ | Mutation/ | AHLTCEGDKKEEASGSNKSGKVHAC |
| | p.V1041A | Peptide | |
| 209 | 8434_FNDC1_ | Mutation/ | ATLRAPRRLSWAVLLLLAALLPVAS |
| | p.A19V | Peptide | |
| 210 | 8434_RGL2_ | Mutation/ | GSPLSGGAEEASEGTGYGGEGSGPG |
| | p.G551E | Peptide | |
| 211 | 8434_TOP1MT_ | Mutation/ | FIDKLALRAGNEEEDGEAADTVGCC |
| | p.K231E | Peptide | |
| 212 | 8434_TMEM55A_ | Mutation/ | KCTVCNEATPIKTPPTGKKYVRCPC |
| | p.N89T | Peptide | |
| 213 | 8434_COL4A5_ | Mutation/ | GDQGLPGDRGPPEPPGIRGPPGPPG |
| | p.G270E | Peptide | |
| 214 | 8434_ATP11C_ | Mutation/ | SARNPNLELPMLFSYKHTDSGYS |
| | p.L1109F | Peptide | |
| 215 | 8434_DCHS2_ | Mutation/ | LIPGNVSSLFTIESTTGLYSPEVEI |
| | p.D228E-2 | Peptide | |
| 216 | 8434_PRRC2C_ | Mutation/ | STSAPVPASPLASVSASASVSASVP |
| | p.P1808S | Peptide | |
| 217 | 6932_KDELC2_ | Mutation/ | NHVYRRSLGKYTGFKMFSDEILLSL |
| | p.D213G | Peptide | |
| 218 | 6932_SLC5A12_ | Mutation/ | QENLENGSARKQEAESVLQNGLRRE |
| | p.G581E | Peptide | |
| 219 | 6932_CMKLR1_ | Mutation/ | KISCFNNFSLSTSGSSSWPTHSQMD |
| | p.P198S | Peptide | |
| 220 | 6932_RIMBP2_ | Mutation/ | ARCRSESDMENEQNSNTSKQRYSGK |
| | p.R173Q | Peptide | |
| 221 | 6932_AKAP3_ | Mutation/ | LAQGGRRDARSFIEAAGTTNFPANE |
| | p.V551I | Peptide | |
| 222 | 6932_ATP11A_ | Mutation/ | VLKRDPTLYRDVTKNALLRWRVFIY |
| | p.A955T | Peptide | |
| 223 | 6932_NDFIP2_ | Mutation/ | GGRGPAATTSSTAVAVGAEHGEDSL |
| | p.G72A | Peptide | |
| 224 | 6932_CSK_ | Mutation/ | LFLVRESTNYPGYYTLCVSCDGKVE |
| | p.D115Y | Peptide | |
| 225 | 6932_IGF1R_ | Mutation/ | RCQKMCPSTCGKQACTENNECCHPE |
| | p.R222Q | Peptide | |
| 226 | 6932_RHBDL1_ | Mutation/ | KRAIANGQRALPWDGPLDEPGLGVY |
| | p.R154W | Peptide | |
| 227 | 6932_HGS_ | Mutation/ | QIMKVEGHVFPEIKESDAMFAAERA |
| | p.F145I | Peptide | |
| 228 | 6932_MAPK4_ | Mutation/ | VDGGASPQFDLDEFISRALKLCTKP |
| | p.V541E | Peptide | |
| 229 | 6932_SERPINB4_ | Mutation/ | VEAAAATAVVVVKLSSPSTNEEFCC |
| | p.E353K | Peptide | |
| 230 | 6932_BTBD2_ | Mutation/ | VFDAMFNGGMATKSTEIELPDVEPA |
| | p.T157K | Peptide | |
| 231 | 6932_ZNF114_ | Mutation/ | AFREDGSLRAHNAHGREKMYDFTQC |
| | p.T241A | Peptide | |
| 232 | 6932_MCOLN1_ | Mutation/ | ESELQAYIAQCQHSPTSGKFRRGSG |
| | p.D546H | Peptide | |
| 233 | 6932_RYR2_ | Mutation/ | NPVEGERYLDFLSFAVFCNGESVEE |
| | p.R2303S | Peptide | |
| 234 | 6932_OR2T10_ | Mutation/ | VGSVDGFMLTPISMSFPFCRSHEIQ |
| | p.A163S | Peptide | |
| 235 | 6932_CC2D1B_ | Mutation/ | KLQYQRAALQAKHSQDLEQAKAYLR |
| | p.R550H | Peptide | |
| 236 | 6932_ZZZ3_ | Mutation/ | AHPEEISSNSQVLSRSPKKRPEPVP |
| | p.R46L | Peptide | |
| 237 | 6932_THBD_ | Mutation/ | LVVALLALLCHLCKKQGAARAKMEY |
| | p.R540C | Peptide | |
| 238 | 6932_HELZ2_ | Mutation/ | NPIHARGKVPPHARHYPLMFCHVAG |
| | p.P775A | Peptide | |
| 239 | 6932_EVX2_ | Mutation/ | GAAQLKENNGKGFAESGSAAGTTTS |
| | p.Y144F | Peptide | |
| 240 | 6932_D2HGDH_ | Mutation/ | SVSGILVCQAGCILEELSRYVEERD |
| | p.V173I | Peptide | |
| 241 | 6932_CD207_ | Mutation/ | GPSLVPGKTPTVCAALICLTLVLVA |
| | p.R43C | Peptide | |
| 242 | 6932_PIK3CA_ | Mutation/ | ALEYFMKQMNDARHGGWTTKMDWIF |
| | p.H1047R | Peptide | |
| 243 | 6932_MTMR14_ | Mutation/ | GAIGGLLEQFARVVGLRSISSNAL |
| | p.G639V | Peptide | |
| 244 | 6932_GABRB1_ | Mutation/ | TLDNRVADQLWVQDTYFLNDKKSFV |
| | p.P119Q | Peptide | |
| 245 | 6932_PCDHAC2_ | Mutation/ | RERQLFSIDASTWEVRVIGGLDYEE |
| | p.G315W | Peptide | |
| 246 | 6932_ZFPM2_ | Mutation/ | LDVTWQGVEDNKKNCIVYSKEDIFP |
| | p.N170K | Peptide | |
| 247 | 6932_ZFPM2_ | Mutation/ | LDVTWQGVEDNKKNCIVYSKGGQLW |
| | p.N170K* | Peptide | |
| 248 | 6932_BCORL1_ | Mutation/ | ANIYPRCSVNGKLTSTQVLPVGWSP |
| | p.P823L | Peptide | |
| 249 | 6932_ZNRF3_ | Mutation/ | GEPWPGPASPSGDAAWR |
| | p.D556fs | Peptide | |
| 250 | 0025_ANO5_ | Mutation/ | SGATVTLWMSLVITSMVAVIVYRLS |
| | p.V634I | Peptide | |
| 251 | 0025_CHST1_ | Mutation/ | RRVMLGASRDLLWSLYDCDLYFLEN |
| | p.R125W | Peptide | |
| 252 | 0025_CCDC88B_ | Mutation/ | KQKLVEKIMDQYHVLEPVPLPRTKK |
| | p.R1300H | Peptide | |
| 253 | 0025_TENM4_ | Mutation/ | TTDIISVANEDGQRVAAILNHAHYL |
| | p.R2592Q | Peptide | |
| 254 | 0025_KRAS_ | Mutation/ | MTEYKLVVVGACGVGKSALTIQLI |
| | p.G12C | Peptide | |
| 255 | 0025_SCAF11_ | Mutation/ | RRQSQSRSPKRDSTRESRRSESLSP |
| | p.T945S | Peptide | |
| 256 | 0025_AMDHD1_ | Mutation/ | AGGGIHFTVERTCQATEEELFRSLQ |
| | p.R127C | Peptide | |
| 257 | 0025_SLC25A29_ | Mutation/ | RGVNRGMVSTLLCETPSFGVYFLTY |
| | p.R94C | Peptide | |
| 258 | 0025_ZFYVE19_ | Mutation/ | SLELDYHTSSCFQGTMVKADCPVPI |
| | p.R60Q | Peptide | |
| 259 | 0025_FGF7_ | Mutation/ | RGKKTKKEQKTAYFLPMAIT |
| | p.H187Y | Peptide | |
| 260 | 0025_MFGE8_ | Mutation/ | SYARLDKQGNFNDWVAGSYGNDQWL |
| | p_A277D | Peptide | |
| 261 | 0025_ZNF276_ | Mutation/ | SMVHPLTQTQDKVLPLEAEPPPGPP |
| | p.A372V | Peptide | |
| 262 | 0025_FBF1_ | Mutation/ | RRENEELSARYLLQCQEAEQARAEL |
| | p.S674L | Peptide | |
| 263 | 0025_RNF157_ | Mutation/ | GTFCVKPLKQKQIVDGVSYLLQEIY |
| | p.V240I | Peptide | |
| 264 | 0025_RNF213_ | Mutation/ | APHKKVGFVGISDWALDPAKMNRGI |
| | p_N2935D | Peptide | |
| 265 | 0025_DDX39A_ | Mutation/ | PSEVQHECIPQAFLGMDVLCQAKSG |
| | p.I79F | Peptide | |
| 266 | 0025_RHPN2_ | Mutation/ | TRQMGLLFTWYDCLTGVPVSQQNLL |
| | p.S201C | Peptide | |
| 267 | 0025_NLRP9_ | Mutation/ | LQRRGDCFAFMHQCIQEFCAAMFYL |
| | p.L446Q | Peptide | |
| 268 | 0025_MTOR_ | Mutation/ | LLANDPTSLRKNFSIQRYAVIPLST |
| | p.L2220F | Peptide | |
| 269 | 0025_LAX1_ | Mutation/ | HATEYAVGIYDNSMVPQMCGNLTPS |
| | p.A158S | Peptide | |
| 270 | 0025_SOX13_ | Mutation/ | PARASQDSADPQTPAQGNFRGSWDC |
| | p.A63T | Peptide | |
| 271 | 0025_MN1_ | Mutation/ | LFGQSCLAALSTGCQNMIASLGAPN |
| | p.A831G | Peptide | |
| 272 | 0025_RPS19BP1_ | Mutation/ | GLELLAASEAPRYPPGQAKPRGAPV |
| | p.D21Y | Peptide | |
| 273 | 0025_KIAA0930_ | Mutation/ | NTFQGVIFQGSICYEALKKVYDNRV |
| | p.R208C | Peptide | |
| 274 | 0025_CMBL_ | Mutation/ | DKPYIDEARRNLTEWLNKYM |
| | p.I238T | Peptide | |
| 275 | 0025_GCNT2_ | Mutation/ | TKYVHQELLNHKKSYVIKTTKLKTP |
| | p.N241K | Peptide | |
| 276 | 0025_ECI2_ | Mutation/ | KDPGNEVKLKLYGLYKQATEGPCNM |
| | p.A37G | Peptide | |
| 277 | 0025_GRM8_ | Mutation/ | CFSYAALLTKTNHIHRIFEQGKKSV |
| | p.R672H | Peptide | |
| 278 | 0025_KLRG2_ | Mutation/ | AGAGLEPSSKKKLPSPRPGSPRVPP |
| | p.P70L | Peptide | |
| 279 | 0025_RPL8_ | Mutation/ | RFKKRTELFIAAKGIHTGQFVYCGK |
| | p.E80K | Peptide | |
| 280 | 0025_GFRA2_ | Mutation/ | LFCSCQDQACAEHRRQTILPSCSYE |
| | p.R246H | Peptide | |
| 281 | 0025_PTCH1_ | Mutation/ | RPHRPEWVHDKAYYMPETRLRIPAA |
| | p.D803Y | Peptide | |
| 282 | 0025_NYX_ | Mutation/ | LRTLNLGGNALDHVARAWFADLAEL |
| | p.R268H | Peptide | |
| 283 | 0025_ARID1A_ | Mutation/ | VKIVQKNDPFVVEISLGVCRSLTVA |
| | p.D1825fs-1 | Peptide | |
| 284 | 0025_ARID1A_ | Mutation/ | KNDPFVVEISLGVCRSLTVACCTGG |
| | p.D1825fs-2 | Peptide | |
| 285 | 0025_ARID1A_ | Mutation/ | VVEISLGVCRSLTVACCTGGLVGGT |
| | p.D1825fs-3 | Peptide | |
| 286 | 0025_ARID1A_ | Mutation/ | LGVCRSLTVACCTGGLVGGTPLSIS |
| | p.D1825fs-4 | Peptide | |
| 287 | 0025_ARID1A_ | Mutation/ | SLTVACCTGGLVGGTPLSISRPTSR |
| | p.D1825fs-5 | Peptide | |
| 288 | 0025_ARID1A_ | Mutation/ | CCTGGLVGGTPLSISRPTSRARQSC |
| | p.D1825fs-6 | Peptide | |
| 289 | 0025_ARID1A_ | Mutation/ | LVGGTPLSISRPTSRARQSCCLPGL |
| | p.D1825fs-7 | Peptide | |
| 290 | 0025_ARID1A_ | Mutation/ | PLSISRPTSRARQSCCLPGLTHPAH |
| | p.D1825fs-8 | Peptide | |
| 291 | 0025_ARID1A_ | Mutation/ | ISRPTSRARQSCCLPGLTHPAHQPL |
| | p.D1825fs-9 | Peptide | |
| 292 | 0025_ARID1A_ | Mutation/ | PTSRARQSCCLPGLTHPAHQPLGSM |
| | p.D1825fs-10 | Peptide | |

The present disclosure also provides recombinant vectors expressing a TCR, or an antigen-binding portion thereof, that are disclosed herein. Production of recombinant vectors is well-known in the art, and a variety of vectors may be utilized, including viral or non-viral vectors.

The present disclosure also provides recombinant vectors comprising a polycistronic expression cassette comprising a transcriptional regulatory element operably linked to a polycistronic polynucleotide. The present disclosure provides recombinant polycistronic nucleic acid vectors comprising at least three cistrons, wherein the first cistron encodes an α chain of an artificial T-cell receptor (TCR), the second cistron encodes a β chain of an artificial TCR, and the third cistron encodes a fusion protein that comprises IL-15 and IL-15Rα (e.g., mbIL15), or a functional fragment or functional variant thereof. In some aspects, the polycistronic nucleic acid further comprises a fourth cistron that encodes a marker protein (e.g., HER1t). In some aspects, the cistrons are separated by polynucleotide sequence that comprise 2A elements. Any of the TCR alpha or beta chain sequences disclosed herein may be used in the recombinant vectors. Non-limiting examples of the 2A element sequences, the IL-15 sequences, and the sequences are known in the art, e.g., as provided in PCT publication WO 2022/183167, which is incorporated by reference herein in its entirety.

In some aspects, the recombinant vector comprises a polycistronic expression cassette, where the polycistronic expression cassette comprises a transcriptional regulatory element operably linked to a polycistronic polynucleotide that comprises: a first polynucleotide sequence that encodes a T cell receptor (TCR) alpha chain comprising an alpha chain variable (Vα) region and an alpha chain constant (Cα) region; a second polynucleotide sequence that comprises a first 2A element; a third polynucleotide sequence that encodes a TCR beta chain comprising a beta chain variable (Vβ) region and a beta chain constant (Cβ) region; a fourth polynucleotide sequence that comprises a second 2A element; and a fifth polynucleotide sequence that encodes a fusion protein that comprises IL-15, or a functional fragment or functional variant thereof, and IL-15Rα, or a functional fragment or functional variant thereof. As provided in PCT publication WO 2022/183167, the recombinant vector may comprise the five polynucleotide sequence in any order from 5′ to 3′.

In some aspects, transgenes of the recombinant vector or any vectors used in the present disclosure are introduced into an immune effector cell via synthetic DNA transposable elements, e.g., a DNA transposon/transposase system, e.g., Sleeping Beauty (SB). SB belongs to the Tc1/mariner superfamily of DNA transposons. DNA transposons translocate from one DNA site to another in a simple, cut-and-paste manner. Transposition is a precise process in which a defined DNA segment is excised from one DNA molecule and moved to another site in the same or different DNA molecule or genome.

Exemplary DNA transposon/transposase systems include, but are not limited to, Sleeping Beauty (see, e.g., U.S. Pat. Nos. 6,489,458, 8,227,432, the contents of each of which are incorporated by reference in their entirety herein), piggyBac transposon system (see e.g., U.S. Pat. No. 9,228,180, Wilson et al, “PiggyBac Transposon-mediated Gene Transfer in Human Cells,” Molecular Therapy, 15:139-145 (2007), the contents of each of which are incorporated by reference in their entirety herein), piggyBac transposon system (see e.g., Mitra et al., “Functional characterization of piggyBac from the bat Myotis lucifugus unveils an active mammalian DNA transposon,” Proc. Natl. Acad. Sci USA 110:234-239 (2013), the contents of which are incorporated by reference in their entirety herein), TcBuster (see e.g., Woodard et al. “Comparative Analysis of the Recently Discovered hAT Transposon TcBuster in Human Cells,” PLOS ONE, 7(11): e42666 (November 2012), the contents of which are incorporated by reference in their entirety herein), and the Tol2 transposon system (see e.g., Kawakami, “Tol2: a versatile gene transfer vector in vertebrates,” Genome Biol. 2007; 8(Suppl 1): S7, the contents of each of which are incorporated by reference in their entirety herein). Additional exemplary transposon/transposase systems are provided in U.S. Pat. Nos. 7,148,203; 8,227,432; US20110117072; Mates et al., Nat Genet, 41(6):753-61 (2009); and Ivies et al., Cell, 91(4):501-10, (1997), the contents of each of which are incorporated by reference in their entirety herein).

In some aspects, the transgenes described herein are introduced into an immune effector cell via the SB transposon/transposase system. The SB transposon system comprises a SB a transposase and SB transposon(s). The SB transposon system can comprise a naturally occurring SB transposase or a derivative, variant, and/or fragment that retains activity, and a naturally occurring SB transposon, or a derivative, variant, and/or fragment that retains activity. An exemplary SB system is described in, Hackett et al., “A Transposon and Transposase System for Human Application,” Mol Ther 18:674-83, (2010), the entire contents of which are incorporated by reference herein.

In some aspects, the recombinant vector comprises a Left inverted terminal repeat (ITR), i.e., an ITR that is 5′ to an expression cassette, and a Right ITR, i.e., an ITR that is 3′ to an expression cassette. The Left ITR and Right ITR flank the polycistronic expression cassette of the vector. In some aspects, the Left ITR is in reverse orientation relative to the polycistronic expression cassette, and the Right ITR is in the same orientation relative to the polycistronic expression cassette. In some aspects, the Right ITR is in reverse orientation relative to the polycistronic expression cassette, and the Left ITR is in the same orientation relative to the polycistronic expression cassette.

In some aspects, the Left ITR and the Right ITR are ITRs of a DNA transposon selected from the group consisting of a Sleeping Beauty transposon, a piggyBac transposon, TcBuster transposon, and a Tol2 transposon. In some aspects, the Left ITR and the Right ITR are ITRs of the Sleeping Beauty DNA transposon.

The present disclosure further provides a population of cells that comprise the recombinant vectors disclosed herein. In one aspect, the recombinant vector or the polynucleotide is integrated into the genome of the population of cells. In one aspect, the cells are immune effector cells. In certain aspects, the immune effector cells are selected from the group consisting of T cells, natural killer (NK) cells, B cells, mast cells, and myeloid-derived phagocytes.

The present disclosure also provides a population of cells comprising a polycistronic expression cassette comprising: a. a first cistron comprising a polynucleotide sequence that encodes a fusion protein that comprises IL-15, or a functional fragment or functional variant thereof, and IL-15Rα, or a functional fragment or functional variant thereof; b. a second cistron comprising a polynucleotide sequence that encodes a TCR beta chain comprising a V3 region and a Cp region; and c. a third cistron comprising a polynucleotide sequence that encodes a TCR alpha chain comprising a Vα region and a Cα region.

In some aspects, the recombinant vectors disclosed herein comprise a polynucleotide sequence that encodes an amino acid sequence at least 90%, 91%, 92%, 93%, 94%, 95%, 96%, 97%, 98%, 99%, or 100% identical to any of the TCR alpha or beta chain sequences provided in Tables 1-3 herein.

The present disclosure provides a pharmaceutical composition comprising a population of cells as disclosed herein. In one aspect, the pharmaceutical composition comprises a pharmaceutically acceptable carrier.

It is contemplated that the TCRs identified by the methods disclosed herein, the antigen-binding portions thereof, populations of cells, and pharmaceutical compositions can be used in methods of treating or preventing medical conditions, such as cancer. Without being bound to a particular theory or mechanism, the TCRs, or the antigen-binding portions thereof, are believed to bind specifically to a mutated amino acid sequence encoded by a cancer-specific mutation, such that the TCR, or the antigen-binding portion thereof, when expressed by a cell, is able to mediate an immune response against a target cell expressing the mutated amino acid sequence. In this regard, an aspect of the disclosure provides a method of treating or preventing cancer in a mammal, comprising administering to the mammal any of the pharmaceutical compositions, isolated pairs of TCR α and β chain sequences, antigen-binding portions thereof, or populations of cells described herein, in an amount effective to treat or prevent cancer in the mammal.

Aspects of the disclosure include a cell or cells encompassed by the disclosure for use in the treatment of a medical condition, such as cancer or a premalignant condition, in a subject. The cells may be used for any type of cancer, including neuroblastoma, breast cancer, cervical cancer, ovary cancer, endometrial cancer, melanoma, bladder cancer, lung cancer, pancreatic cancer, colon cancer, prostate cancer, hematopoietic tumors of lymphoid lineage, leukemia, acute lymphocytic leukemia, chronic lymphocytic leukemia, B-cell lymphoma, Burkitt's lymphoma, multiple myeloma, Hodgkin's lymphoma, Non-Hodgkin's lymphoma, myeloid leukemia, acute myelogenous leukemia (AML), chronic myelogenous leukemia, thyroid cancer, thyroid follicular cancer, tumors of mesenchymal origin, fibrosarcoma, rhabdomyosarcomas, melanoma, uveal melanoma, teratocarcinoma, neuroblastoma, glioma, glioblastoma, benign tumor of the skin, renal cancer, anaplastic large-cell lymphoma, esophageal squamous cells carcinoma, hepatocellular carcinoma, follicular dendritic cell carcinoma, intestinal cancer, muscle-invasive cancer, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, prostatic hypertrophy, myelodysplasia, Waldenstrom's macroglobinaemia, nasopharyngeal, neuroendocrine cancer myelodysplastic syndrome, mesothelioma, angiosarcoma, Kaposi's sarcoma, carcinoid, oesophagogastric, fallopian tube cancer, peritoneal cancer, papillary serous mullerian cancer, malignant ascites, gastrointestinal stromal tumor (GIST), or a hereditary cancer syndrome selected from Li-Fraumeni syndrome and Von Hippel-Lindau syndrome (VHL).

The examples of the present disclosure are offered by way of illustration and explanation, and are not intended to limit the scope of the present disclosure.

### EXAMPLES

### Example 1: Workflow to Identify Tumor Specific TCRs from Patient Derived Til and Dissociated Primary Tumors

### 1.1 TCR Identification and Screening Platform

The fundamental basis for this unbiased TCR identification and screening platform is illustrated in FIG. 1. Initially, single-cell gene expression data (e.g., 5′ GEX Analysis) from T cells is utilized to perform unsupervised clustering analysis by employing dimensionality reduction methods such as principal component analysis (PCA), t-distributed Stochastic Neighbor Embedding (tSNE), or Uniform Manifold Approximation and Projection (UMAP) (FIG. 1, STEP 1). Merging the clustered single-cell gene expression analysis with paired, full-length TCR sequences then enables the identification of TCR clonotypes present in each of the distinct clusters. TCR sequences are then selected from the overall single-cell dataset based on frequency, cluster attributes, specific-gene expression signatures, or other criteria employed to increase the likelihood of obtaining TCRs with desired reactivity (i.e., antigen/HLA specificity) (FIG. 1, STEP 2). Selected paired, full-length TCR sequences are then reconstructed in silico, from which, expression plasmids encoding the TCR α and β chains synthesized (FIG. 1, STEP 3). These TCR expression cassettes are then cloned into transposon or other non-viral gene transfer vector to enable quick translation into process development, manufacturing, and clinical applications. TCR-expression plasmids are then transiently expressed in a cell line (e.g., Jurkat or SUP-T1) or primary cell (e.g., human ex vivo expanded T cells) that will signal upon TCR recognition of cognate antigen:HLA complexes on the surface of antigen presenting cells (APCs) (FIG. 1, STEP 4). Antigen presenting cells (APCs) are classical professional APCs such as dendritic cells (DCs) or an artificial antigen presenting cell (e.g., COS-7 or 293-HEK). APCs either endogenously express the requisite HLA allele(s) or are transfected with HLA expression plasmids. Antigens are introduced to the APCs either by genetic transfer to antigen encoding plasmids (e.g., Tandem Minigene (TMG) plasmids) or by the pulsing of peptide pools. One aspect of the APC system used is that multiple HLA alleles and antigens are screened within the same set of APCs, thus enable high-throughput assessment of hundreds to thousands of antigen:HLA combinations. Co-culture of the TCR modified cells and APCs is then performed to identify reactive TCRs (FIG. 1, STEP 5). Reactive TCRs are those that are found to recognize one of the antigen:HLA conditions tested. These reactive TCRs are then further evaluated in vitro to confirm the findings and deconvolute the multiplexed HLA/antigen. Once all reactive TCRs are identified from a specimen, that binary outcome (reactive vs non-reactive) for each TCR can be mapped back to the initial gene-expression cluster analysis (FIG. 1, STEP 6). By mapping the reactive TCRs back to the gene-expression data, gene signatures or biomarkers which are enriched in the reactive TCR cell population are elucidated and used to further improve and refine the initial selection of TCRs for screening. Overall, this fundamental process is used to identify TCR sequences and their associated antigen and HLA specificity with a high level of confidence and accuracy from complex starting materials such as tumor tissues or blood samples.

1.2 Screening of TCRs from TILs

In practice, the steps of the above-described workflow (FIG. 1) can be further broken down into critical processes as shown in FIG. 2 for screening of TCRs obtained from TILs. The process illustrated in FIG. 2 correspond to FIG. 1 STEPs 1-5. The workflow illustrated in FIG. 2 is further broken into two parallel processes (indicated with either Alpha [i.e., A, B, C, etc.] or Numeric [i.e., 1, 2, 3, etc.] STEP designators) that diverge from a common starting point (STEP 1/A) and converge at a common finishing point (STEP 8/F). STEP 1/A to STEP 6 illustrate the workflow from TILs isolation to generation of cells expressing TILs-derived TCRs. STEP 1/A to STEP D illustrate the workflow from patient mutation and HLA calling to the generation of APCs expressing the patient matched HLA and mutation-derived antigens (e.g., neoantigens).

STEP 1-6 (TCR): Initially, in STEP 1/A, a tumor sample is obtained from a cancer patient (FIG. 2). This tumor sample is dissociated into a single-cell suspension and TILs are isolated by fluorescent activated cell sorting (FACS) by staining dissociated tumor samples for lymphocyte, T cell, and live cell markers (FIG. 2, STEP 2). Single-cell transcriptomics is then performed on the sorted TILs to obtain gene expression and TCR V(D)J sequences (FIG. 2, STEP 3). Bioinformatic analysis of the gene-expression data is used to cluster cells based on transcriptional similarities to aid in the selection of TCR sequences for in vitro evaluation (FIG. 2, STEP 4). Once selected, TCRs are reconstructed in silico and synthesized in expression vectors (FIG. 2, STEP 5) to enable transgenic expression of the TCRs in cells capable of forming a functional TCR complex with CD3 subunits and CD4/CD8 co-receptors. These cells are engineered to express any or all necessary protein components of the TCR signaling complex or downstream signaling components. Moreover, these components are modified to further enhance their function in the platform (e.g., CD4 with amino acid substitutions at Q40Y, T45W, P48L, S60R, and/or D63R to enhance affinity to MHC-Class II). Wang et al. 2011 PNAS, 108(38): 15960-15965. TCR expression vectors are transferred into the Reporter cells to generate Reporter TCR-T cells (FIG. 2 STEP 6).

STEP A-D (Antigen/HLA): In parallel to STEPs 1-6, nucleic acids (DNA and RNA) can be extracted from the tumor sample (FIG. 2, STEP 1/A). Using Whole Exome Sequencing (WES) and RNA Sequencing (RNAseq) to generate genomic and transcriptional datasets, a bioinformatics pipeline is employed to determine somatic mutations present in the tumor as well as the patient's germline HLA typing (FIG. 2, STEP B). Somatic mutations are ranked and concatenated so that TMGs and peptide pools can be synthesized (FIG. 2, STEP C). These reagents provide the antigen component of the screening assay. Similarly, sequences of the called HLA alleles are synthesized in expression vectors to provide the HLAs necessary for the screening assay. Antigen presenting cells, such as COS-7, are then modified either by stable or transient transfection to express the requisite Class I or Class II HLA alleles either in single-plex or multiplexed within the same cells (FIG. 2, STEP D). Antigen is provided to the APCs either by transfection of relevant TMGs (either as plasmid DNA or in vitro transcribed RNA) and/or peptide pools containing antigens derived from the tumor's somatic mutations identified. With both the HLA and antigen provided to the APCs, they are able to present peptide:HLA complexes to T cells in vitro.

STEP 7/E-8/F: Reporter cells expressing transgenic TCRs (FIG. 2, STEP 6) and antigen/HLA-modified APCs (FIG. 2, STEP D) are co-cultured together at a pre-determined ratio of Reporter cells (E) to APCs (T), typically approximately 4:1 to 8:1 (FIG. 2, STEP 7/E). Positive control wells containing PMA/Ionomycin or coated with H57-597 antibody (anti-transgenic TCR) with the TCR-modified Reporter cells are also set up. Negative control wells of Reporter cells alone or co-cultured with APCs modified with HLA-only, irrelevant antigens, or non-transfected are also set up. All conditions are typically evaluated in duplicate. After the co-culture period, reporter activity (i.e., luciferase activity) is quantified in each co-culture and control well (FIG. 2, STEP 8/F). For a given TCR, the reporter activity is compared across all antigen:HLA conditions evaluated to determine if there is a condition with increased reporter activity which indicates that the transgenic TCR recognized an antigen:HLA combination present in that well. Because initial screening multiplexes multiple HLA alleles and antigens, when there is specific TCR activity observed, STEP 7/E and 8/F are repeated using APCs modified with single HLA and antigens to elucidate the exact specificity of the TCR. Moreover, minimal epitopes can be determined using this co-culture method. Overall, this workflow enables the identification of TCR sequences and the empirical determination of specificity to selected antigens and HLA alleles.

### 1.3 Relationship Between TCR-Based and TILs-Based Screening Methods

FIG. 3 illustrates the relationship between a TCR-based screening method (below dotted line) and TILs-based screening method (above dotted line). The TCR-based screening method is as described above in the description of FIG. 2 wherein TCR sequences, somatic mutations, and HLA-typing is obtained from primary tumor samples and utilized to screen selected TCRs for reactivity to tumor neoantigens using a co-culture reporter system. Similarly, TILs screening starts with a primary tumor sample obtained from a cancer patient. TILs are expanded from the tumor using standard TILs expansion methods (high-concentration IL-2, feeder cells, muromonab-CD3 (OKT3)). Expanded TILs are then co-cultured in an IFN-γ ELISpot with APCs modified to express the relevant HLA alleles and antigens identified from WES and RNAseq data from the tumor. This is performed in a similar plate layout to TCR screening where multiple HLA alleles and antigens are multiplexed in the same wells, thus increasing the throughput of the assay. Positive controls include PMA/Ionomycin. Negative controls include TILs alone, APCs alone, TILs+APCs without HLA and/or antigen, and no cells. After the overnight co-culture, cells are harvested from the IFN-γ ELISpot and the plate is developed to measure the number of spot-forming colonies (SFCs) of each well. The harvested TILs are also stained and evaluated for upregulation of 4-1BB or other activation molecules (e.g., OX40). TILs from co-culture conditions which produce increased numbers of SFCs and/or activation marker expression are then sorted for either total live T cells or for T cells expressing the activation marker. Single cell gene expression and TCR V(D)J sequencing is then performed on the sorted cells. T cells from a negative control co-culture (typically APCs modified with HLA alone or with HLA and irrelevant antigen) are similarly sorted and analyzed by single-cell transcriptomics. Using the single-cell gene expression data, clusters of activated TILs can be identified. Paired, full-length TCR sequences from these activation clusters are then reconstructed into TCR expression plasmids and screened using the TCR screening methods described in FIG. 2. Overall, FIG. 3 illustrates parallel workflows with either ex vivo expanded TILs or sorted TILs are utilized to identify tumor-reactive TCRs with potential therapeutic applications in oncology. These general methods are applied to identify therapeutically useful TCRs in other disease indications (e.g., inflammation, auto-immune, etc.) with the appropriate starting material (e.g., a biopsy of inflamed colon from Crohn's disease patient or a plaque of a patient with psoriasis).

### Example 2: Development of TCR Screening Methods

### 2.1 General Methods Used in the Examples

### 2.1.1 Nucleic Acid Isolation and Assessment

Tumor samples are obtained as either dissociated tumors or frozen tissue. To isolate DNA and RNA from dissociated cells, cells are processed using Qiagen AllPrep DNA/RNA Mini kit per the manufacturer's protocol. To isolate DNA and RNA from tissue, frozen tissue is disrupted using a mortar and pestle and homogenized using QIAshredder homogenizers. The homogenized tissue is processed through the Qiagen AllPrep DNA/RNA Mini kit according to the manufacturer's protocol.

Matched normal samples are obtained either as whole blood or as PBMCs. The Qiagen DNeasy Blood & Tissue kit is used to isolate DNA from 200 μL of whole blood per the manufacturer's protocol and including the optional RNaseA. To isolate DNA and RNA from PBMCs, cells are processed using Qiagen AllPrep DNA/RNA Mini kit per the manufacturer's protocol.

Isolated nucleic acids are quantified by fluorescence spectrometry using the Life Technologies Qubit dsDNA BR Assay kit. Nucleic acids are assessed for fragment size by automated electrophoresis using the Agilent TapeStation 4150. Genomic DNA is assessed using the Agilent Genomic DNA ScreenTape System and RNA is assessed using the Agilent RNA ScreenTape System.

### 2.1.2 RNAseq

To assess gene expression, RNA from tumors are processed through Illumina RNA Prep with Enrichment with an input of 100 ng. Pre-capture libraries are enriched via hybridization with the Illumina Exome Panel.

Molarity of final libraries is determined using the size for fragments between 100 and 1000 bp on the Agilent TapeStation 4150 (Agilent High Sensitivity D1000 ScreenTape assay) and library concentration from Qubit 4 (Life Technologies Qubit dsDNA BR Assay kit). Libraries are pooled with a 1% PhiX spike-in. The library pool is clustered and sequenced at 2×76 on an Illumina NextSeqDx 550 using a 150 cycle High Output kit for a target coverage of 150 M reads. Libraries are subject to on-board demultiplexing to yield FASTQ files.

The raw RNA-seq reads are aligned to the hg19 genome using Spliced Transcripts Alignment to a Reference (STAR) with the two-step procedure. Then Cufflinks is applied to the obtained BAM file to calculate the Fragments Per Kilobase of transcript per Million mapped reads (FPKM) value of each gene. The FPKM values are converted to deciles to represent ten gene expression levels.

### 2.1.3 Single Cell RNAseq

To sequence TCRs, dissociated tumor cells are processed through the Chromium Next GEM Single Cell 5′ Reagent Kit v2 from 10× Genomics targeting 10,000 cells when possible. The resulting cDNA is processed through the Chromium Single Cell Human TCR Amplification Kit VDJ per manufacturer's recommendations.

Molarity of final libraries is determined using the size for fragments between 100 and 1000 bp on the Agilent TapeStation 4150 (Agilent High Sensitivity D1000 ScreenTape assay) and library concentration from Qubit 4 (Life Technologies Qubit dsDNA BR Assay kit). Libraries are pooled with a 1% PhiX spike-in. The library pool is clustered and sequenced at 26+96 on an Illumina NextSeqDx 550 using a 150 cycle High Output kit for a target coverage of 5000 reads per cell for VDJ and 20,000 reads per gene expression library. Raw bcl files are yielded.

### 2.1.4 Analysis of the 10× Gene Expression (GEX) and VDJ Sequencing Data

The GEX and VDJ sequencing data are preprocessed using the CellRanger toolkit (version 5.1) provided by 10× Genomics. The BCL files from the Illumina sequencer are converted to raw FASTQ files. The FASTQ files for the GEX and VDJ experiments are processed separately. GEX reads are aligned to the human GRCh38 reference genome. Cell barcodes assignment and UMI counting are then performed to create a single-cell gene expression matrix. Doublets and cells with >10% mitochondria gene counts are filtered out in the study. Then the raw read counts are normalized and scaled using Seurat. About 2,000 highly variable genes are identified using the FindVariableGenes module. Next, principal component analysis (PCA) and uniform manifold approximation and projection (UMAP) are performed for dimension reduction and a shared nearest neighbor (SNN) algorithm is applied to cluster the cells.

The raw VDJ reads are assembled into contigs using a graph-based algorithm with the aid of the pre-built reference sequence from the IMGT database. Cells with identical productive V(D)J transcripts are placed into a same clonotype. 2.1.5 Integrating the 10× GEX and VDJ data

For each TCR clonotype, the corresponding cells in V(D)J are projected to the identified clusters in the GEX data. The full-length FASTA sequences of both the TRA and TRB chains, as well as the amino acid sequences of the CDR3 regions for each clonotype are also reported.

### 2.1.6 Whole Exome Sequencing

Whole exome sequencing experiments (WES) are performed for the peripheral blood and the tumor tissue of each patient. Somatic single nucleotide variants (SNVs), short insertions and deletions (indels), copy number alterations (CNAs), class I and II HLA types are detected by comparing the tumor versus the normal sequencing data. Each mutant peptide is predicted in silico if it can give rise to a neoantigen. Bulk RNA-Seq is also performed on the tumor tissue to quantify the expression level of each gene.

Between 100 and 200 ng of genomic DNA is fragmented enzymatically for 100 bp reads using the Agilent SureSelect Enzymatic Fragmentation Kit. Fragmented DNA is processed through the SureSelect XT HS2 DNA System using v7 probes.

Pre-capture libraries the size for fragments between 100 and 1000 bp on the Agilent TapeStation 4150 (Agilent High Sensitivity D1000 ScreenTape assay) and library concentration from Qubit 4 (BR). A total of 1000 ng of pre-capture library is input into hybridization.

Molarity of final libraries is determined using the fragment size between 100 and 1000 bp on the Agilent TapeStation 4150 (Agilent High Sensitivity D1000 ScreenTape assay) and concentration from Qubit 4 (Life Technologies Qubit dsDNA HS Assay Kit). Libraries are pooled with a 1% PhiX spike-in. The library pool is clustered and sequenced at 2×101 on an Illumina NextSeqDx 550 using a 300 cycle High Output kit for a target coverage of 200× and 100× for tumor and normal libraries, respectively. Libraries are subject to on-board demultiplexing to yield FASTQ files.

### 2.1.7 Jurkat NFAT Cell Generation

Jurkat NFAT cells are infected with Lentivirus (pGenLenti-CD8A_P2A_CD8B_IRES_Puro) and then selected with puromycin (0.2 g/mL). Peripheral Blood Mononuclear Cells (PBMCs) from 3 different donors are irradiated and seeded in a 96 multiwell U bottom plate at 100 k/well. Puromycin selected stable pools of peptides are seeded at 0.5 cell/well on top of irradiated PBMCs (96 multiwell plates) to generate single clones. Single clones are cultured for 1 week with IL-2 (50 IU/mL) and Phytohaemagglutinin-L (PHA-L) (0.25 g/mL). Second week cell medium is replaced with 100 IU/mL of IL-2. Grown back clones are evaluated for higher CD3/CD8 expression and higher luciferase signal/noise ratio (PMA/Ionomycin vs untreated). Clone #41 (having >95% CD8 expression and >150 signal to noise ratio) is selected. In order to better screen class II TCRs, #41 clone is infected with CD4 lentivirus (pGenLenti-CD4_IRES_Puro) to boost CD4 expression. After lentivirus infection CD4 expression is increased to more than 95%.

### 2.1.8 Mutation Calling, HLA-Typing and Neoantigen Prediction

The raw WES reads are aligned to the human hg19 reference genome using Burrows-Wheeler Aligner (BWA) (version 0.7.5a). Duplicate reads are marked using Picard's “MarkDuplicates” module. The “IndelRealigner” and “BaseRecalibrator” modules of the Genome Analysis Toolkit are then applied to the obtained BAM files for indel realignment and base quality recalibration. In our workflow, five mutation detection algorithms are applied to the obtained BAM files: Mutect, MuSE, Varscan2, Mutect2 and Strelka, where all of them are used to detect single nucleotide variants (SNVs) and the last three are used to detect short insertions or deletions (indels). Only Mutect2 is used to detect multi-nucleotide variants (MNVs). An SNV is reported if it can be detected by at least three out of the five algorithms. An indel is reported if it can be detected by any of the indel callers.

The detected SNVs are annotated with ANNOVA and VEP and compared with public databases such as dbSNP (Sherry et al., 2001), 1,000 genome (http://www.1000genomes.org/) and ESP6500 (http://evs.gs.washington.edu/EVS/). To ensure accuracy, the following criteria is used to filter the SNV and indel list: allele frequency (AF) >0.05; the coverage is at least 20 reads for the tumor and 10 for the normal; the AF from the normal sample <0.02. Only non-synonymous SNVs, in-frame and frameshift indels are kept for further analysis, as these mutations change the amino acid sequences of the genome and are likely to give rise to neoantigens. For each mutated amino acid that results from a somatic SNV or indel, up to 12 bases are extended to the left and to the right and a peptide sequence of length at most 25 bases (25-mer) is obtained. Since a neoantigen's length ranges from 8-25 bases, it ensures that any potential neoantigen resulting from the mutation is a subsequence of the 25-mer.

The Sequenza algorithm is used to detect the somatic copy number alterations (CNAs) and tumor purity. Optitype and HLA-VBSeq are applied to infer the class I and II HLAs respectively.

The 25-mer peptide sequences and the HLA types of each patient are input together to netMHCpan4.1 to predict if the mutant amino acids can lead to a neoantigen.

### 2.1.9 TCR Plasmid Assembly

Approximately 50 T Cell Receptors (TCRs) are selected per patient by a still-developing method according to their abundance in the assessed sample and the association of their corresponding cells with clusters according to gene expression. TCRs are selected considering whether (1) a cluster expresses CD8 or CD4, (2) the function of genes differentially expressed by that cluster, and (3) the abundance of each TCR. Each analysis yields more than 1000 TCR clonotypes, and these are reduced to approximately 50 clonotypes to move on to synthesis. Each cluster is defined by differentially expressed genes. Each cluster is made up of cells, and each cell is associated with a TCR clonotype. The highest abundance clonotypes in every cluster are included such that a total of approximately 50 clonotypes are synthesized across all clusters, giving preference to clonotypes from clusters that are associated with immune response genes. Similarly, if a patient sample has a Class I or Class II HLA allele that is common in the population, preference is given to clusters that more highly express either CD8 or CD4, respectively.

### 2.1.10 Create Beta and a Gene Sequences in Silico

The raw beta sequence is curated such that any sequence 5′ of the start of the Variable (V) region is replaced with a NheI site, and the entire constant region is replaced with a BspI site. For the α chain, the sequence 5′ of the start of the V region is replaced with an XmaI site, and the constant region is replaced with a SacII site.

Rare codons (defined as codons used <10% according to the Homo sapiens codon usage table) are replaced with more frequently used codons for the same amino acid throughout the beta and a open reading frames. NheI, BspI, XmaI, and SaclI restriction sites are eliminated from the open reading frame by replacing codons with other codons encoding the same residues.

### 2.1.11 Plasmid Assembly

Each α and β gene are synthesized and subcloned into pZT2 plasmids using the synthesized restriction sites (NheI and BspEI for beta and XmaI and SacII for a) by GenScript. The final plasmid is prepared in 10 mMTris-HCl, pH 8.0, 1 mM EDTA (TE) with 95%±5% supercoiled plasmid and ≤0.005 EU/μg endotoxin content.

### 2.1.12 Tandem Minigene Plasmid Assembly

When more than 150 non-synonymous mutations are reported for a tumor, the mutations are sorted by gene expression and only the top 150 expressed non-synonymous mutations are included.

### 2.1.13 TMG Assembly in Silico

Amino acid sequences are reverse translated in silico and codon optimized for expression in human cells. BamHI, EcoRI, NotI and NheI restriction sites are removed by replacing codons with others encoding the same residues. A set of up to 15 sequences are concatenated together into one open reading frame called a tandem minigene (TMG). The nucleotide sequence GAG AAT TCG (codes for Glu (E)/Asn (N)/Ser (S)) and has EcoRI site=GAATTC) is added to the 5′ end of the TMG gene, and the nucleotide sequence AAG GAT CCC (codes for K/D/P and has BamHI site=GGATCC) is added to the 3′ end of the TMG gene.

### 2.1.14 TMG Plasmid Synthesis

The TMG, together with the added restriction sites, is synthesized and cloned (GenScript) into masterTMG_pcDNA3.1(+) mammalian expression vector with EcoRI (5′) and BamHI (3′) in frame with existing start and stop codons. The final plasmid is prepared in TE with 95%±5% supercoiled plasmid and ≤0.005 EU/μg endotoxin content.

### 2.1.15 Peptide Design and Synthesis

The same amino acid sequences are synthesized up to 25 aa in length with crude quality (GenScript). For peptide sequences longer than 25 residues, multiple peptides of 25 aa in length are synthesized with start sites at 5 aa intervals. For the last window, the last 25 residues are synthesized in place of a peptide shorter than 25 aa.

### 2.1.16 Human Leukocyte Antigen (HLA) Plasmid Assembly

Peptide sequences for each identified allele are downloaded from the IPD-IMGT/HLA Database (ebi.ac.uk). Each peptide sequence is reverse translated in silico and codon optimized for expression in human cells. The sequence is then synthesized with BamHI and Kozak sites at the 5′ end and an EcoRI and stop codon on the 3′ end (GenScript). The synthesized sequence is cloned into pcDNA3.1(+) using BamHI and EcoRI. Final plasmids are prepared in TE with 95%±5% supercoiled plasmid and ≤0.005 EU/μg endotoxin content.

### 2.1.17 Neoantigen Specific TCR Screening Process.

On Day 1, COS-7 cells are seeded at 20,000 cells per well (96 multiwell plate) overnight in 37° C. incubator. On Day 2, cell medium is replaced with antibiotic-free DMEM medium before transfection. 150 ng of tandem minigene (TMG) and 300 ng HLA plasmids are transfected using lipofectamine 2000. Three to four HLA plasmids (75-100 ng each) are transfected together in one well to enhance screen efficacy. Each condition includes one or two HLA types including A, B, C, DP, DQ and DR. Twenty-five L of OptiMEM medium is used to dilute either DNA plasmid (450 ng total) or Lipofectamine (0.6 μL) for each well. DNA tube (A) or lipofectamine tube (B) are mixed well separately and incubated for 5 minutes at room temperature (RT). Tube B is then added to tube A, and the mixture is incubated for 20 minutes at RT. Transfection mix (50 μL) is added to each well and cells are cultured overnight in a 37° C. incubator.

Jurkat NFAT reporter cells are counted and seeded at 1 million/mL with fresh RPMI1640 complete medium overnight to enhance electroporation efficacy (10% Fetal Bovine Serum (FBS) and 1% Pen/Strep). On Day 3, Neon™ transfection system is set up in the Biosafety Cabinet (BSC) with program 1,325v, 10 mins, 3 Pulse. 5 mLs of RPMI without Pen/Strep is added into T25 flask and labeled with corresponding murine-TCR (mTCR) number. Flasks are pre-warmed in 37° C. incubator while preparing electroporation (EP) Jurkat NFAT cells are spun down at 100 g for 10 minutes. Cells are washed with PBS and cell numbers are measured with NC3000. 6 million Jurkat NFAT cells are loaded into 15 mL conical tubes and spun down at 100 g for 10 minutes. During centrifugation, Buffer R (110 μL each) are prepared in Eppendorf tubes and Electrolytic Buffer E2 (3 mL each) are aliquoted in Neon transfection system tubes. Eleven microliters of mTCR plasmids (2 mg/mL) are added to corresponding Eppendorf tubes containing Buffer R and mixed well. The mixture of DNA and Buffer R is loaded to the Neon tubes using specialty Neon pipette tips. When EP is successful, “COMPLETE” shows on the screen in a few seconds after “START” is clicked. Buffer R/DNA mixture is transferred immediately into a T25 flask containing antibiotic-free RPMI medium. H57-597 antibody is utilized to coat plate (1 g/mL, L/well) overnight to measure EP efficacy next day. For parsing experiment, peptide is prepared at 50 mg/mL and pulsed at 10 g/mL to identify neoantigen specificity.

### 2.1.18 Co-Culture

On Day 4, Jurkat NFAT-mTCR cells are counted and co-cultured (100 k/well) on top of transfected COS-7 cells for 4-5 hours. As control, Jurkat NFAT-mTCR cells were also plated on H57 coated plate to perform mTCR functional test. After 4-5 hours incubation, cells from 96 multiwells were transferred to U bottom plates and spined down at 400 g for 5 minutes. Cells were then lysed with 1× passive lysis buffer (100 μL/well) for 15 minutes on an orbital shaker at RT. 50 μL cell lysis were loaded onto OPTIPLATE as well as 100 μL of Promega Luciferase substrate. Luciferase activity was measured immediately with BioTek reader. Jurkat NFAT cells mTCR expression was measured with flowcytometry using antibody cocktail CD3, CD4, CD8A, CD8B and H57. HLA expression of COS-7 cells was measured with flowcytometry using antibody cocktail HLA-A2, HLA-DP, HLA-DQ and HLA-DR. 2.1.19 Neoantigen specific Tumor Infiltrating Leukocytes (TILs) identification process. On Day 1, COS-7 cells are seeded at 20,000 cells per well (96 multiwells) overnight in 37° C. incubator. TILs are thawed and recovered with IL-2 at 3000 IU/μL. On Day 2, cell medium is replaced with antibiotic-free DMEM medium before transfection. A Transfection Mix containing 150 ng of tandem minigene (TMG) and 300 ng HLA plasmids are prepared and transfected into the COS-7 cells using lipofectamine 2000. Two HLA plasmids (150 ng each) are transfected together in one well to enhance screening sensitivity. Each condition only includes one HLA type (A, B, C, DP, DQ and DR). 25 μL of OptiMEM medium is used to dilute either DNA plasmids (450 ng total) or Lipofectamine (0.6 μL) for each well. DNA tube (A) and lipofectamine tube (B) are mixed well and incubated separately for 5 minutes at room temperature (RT). Tube B is added to tube A, and the mixture is incubated for 20 minutes at RT. Transfection mix (50 μL) is added to each well and cells area cultured overnight in a 37° C. incubator. On Day 3, 96 multiwell plates containing COS-7 cells are replaced with fresh medium containing peptide pools. Peptide pools are created by combining the peptides from a given TMG into a pool of equivalent mass ratios of each peptide. Peptides are prepared at 50 mg/mL and pulsed at a final concentration of 10 g/mL (in well which contains media and COS-7 cells). Peptide pools consist of the synthesized peptides that correspond to the minigenes within a given TMG (i.e., if TMG-1 contains minigenes encoding Peptide 1, Peptide 2, and Peptide 3, a peptide pool containing Peptides 1-3 would be prepared). ELISpot plates are incubated with 70% EtOH (0.22 m filter, 50 μL/well) for less than 2 mins in the Biosafety Cabinet (BSC) at RT. ELISpot plates are washed 5 times with 200 μL/well with sterile PBS. Anti-interferon gamma capture antibody (1-D1K) is mixed with PBS (100 μL/10 mL/plate) and added 100 μL/well. COS-7 cells are incubated overnight at 4° C. On Day 4, ELISpot plates are washed 5 times with PBS (200 μL/well). Plates are blocked with complete RPMI media (10% FBS), 100 μL/well at room temperature for 1 hour. During the one hour, COS-7 cells are harvested from 96 multiwells using trypsin. TILs are counted and resuspended at 400 k/mL. Medium is poured out from the ELISPOT plate. 50 μL of medium, 100 μL of COS-7 cells, and 100 μL of TILs (40,000 cells) are added sequentially to the ELISpot plates. Plates are transferred to 37° C. incubators with 5% CO₂, and incubated for 18-24 hours. On Day 5, the following ELISpot reagents are prepared: 1) IFN-γ biotinylated 7-B6-1 antibody diluted in PBS+0.5% FBS, then filtered with 0.22 μm filter, and 2) wash buffer (PBS+0.05% Tween-20). Cells of each well are mixed via pipetting, then 200 μL of cells are carefully transferred from ELISpot plate to a new 96 U-bottom plate. The cells are later stained for phenotyping using cocktail CD3, CD4, CD8 and 41BB with flow cytometry. ELISpot plates are washed 3 times using buffer made by combining PBS with 0.05% tween 20 in the big basin. Anti-IFN-γ antibody (Biotinylated 7-B6-1 biotin) is diluted with PBS and 0.5% FBS then filtered with 0.22 m filter (10 μL/10 mL/plate, 100 μL/well). Plates are left at room temperature for 2 hours in the dark covered with aluminum foil. Plates are washed 5 times using PBS with 0.05% tween 20. Streptavidin-ALP is diluted in PBS with 0.05% FBS (10 μL/10 mL) and added at 100 μL/well at room temperature for 1 hr in the dark covered with aluminum foil. Plates are washed 5 times with PBS. 5-Bromo-4-chloro-3-indonyl phosphate, X-phosphate, XP, Nitro-blue-tetrazolium chloride, (BCIP/NBT) Alkaline Phosphatase substrate solution is filtered (0.45 μm) and added at 100 μL to every well. Plates are incubated at room temperature for 10-20 mins until distinct spots can be seen. Tap water is used to wash the plates gently but extensively, then the plates are left out until completely dry. Plates are analyzed using the ELISpot reader. HLA expression of COS-7 cells are measured with flowcytometry using antibodies cocktail HLA-A2, HLA-DP, HLA-DQ and HLA-DR.

### 2.2 Modification of Jurkat Reporter Cells

### 2.2.1 Adding CD8 and CD4

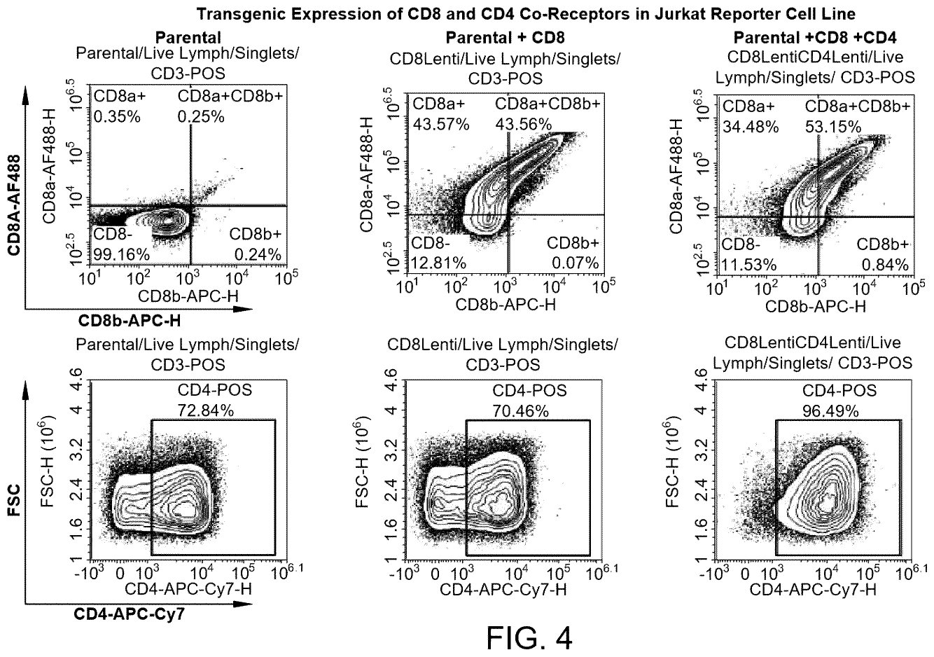

Lentivirus are prepared using HEK-293Ta cells and Jurkat NFAT cells are transduced. Jurkat NFAT cells are first transduced with CD8 Lentivirus and selected with 0.2 pg/ml puromycin to generate Jurkat NFAT_CD8Lenti cells. Subsequently, Jurkat NFAT_CD8Lenti cells are infected with CD4 Lentivirus and selected with 0.3 pg/ml puromycin. After 4 days selection with 0.3 pg/ml puromycin is adjusted back to 0.2 pg/ml for maintenance. Cells are harvested and stained with CD3, CD4, CD8A and CD8B. Jurkat NFAT parental cells are negative for CD8 (99.16% CD8 negative) within the CD3+ cell population. Results shown in FIG. 4 demonstrate that Jurkat LentiCD8 cells have 43.57% CD8A expression and 43.56% CD8A and CD8B double positive expression.

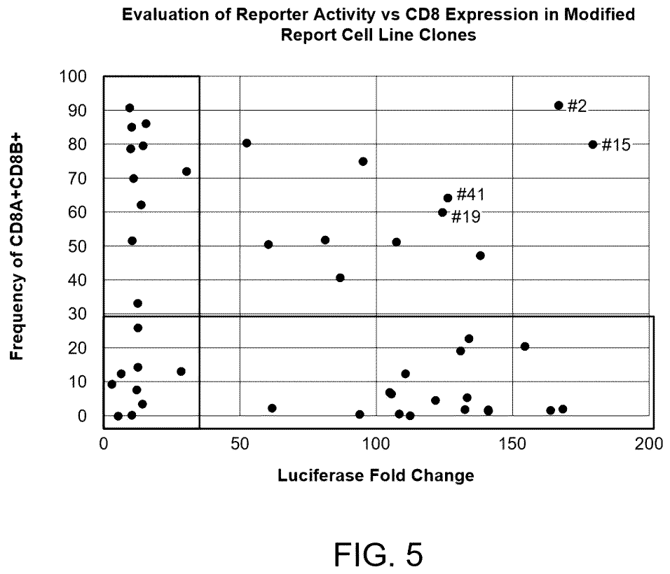

Single clones are generated from Jurkat NFAT_CD8Lenti pool. Peripheral Blood Mononuclear Cells (PBMCs) from 3 different donors are irradiated and seeded in 96-multiwell U bottom plates at 100 k cells/well. Puromycin selected Jurkat NFAT_CD8Lenti stable pool cells are seeded at 0.5 cell/well on top of irradiated PBMCs to generate single clones. Single clones are cultured for one week with IL-2 (50 IU/ml) and phytohaemagglutinin (PHA; 0.25 pg/ml). During the second week cell medium is replaced with 100 IU/ml of IL-2. Grown back clones are evaluated for CD8A and CD8B expression and luciferase signal/noise ratio (PMA/Ionomycin vs untreated). Clones 2, 15, 19, 41 (>95% CD8 expression and >150 signal to noise ratio) are the best clones with higher CD8 expression and higher luciferase activity signal to noise ratio (FIG. 5).

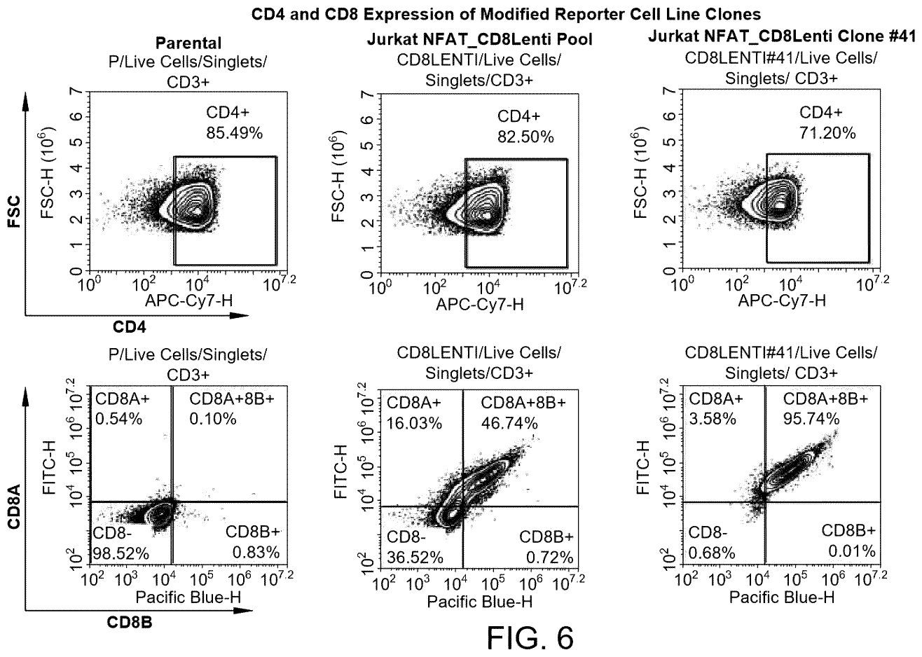

To better improve screening efficacy, clone #41 is selected from the Jurkat NFAT CD8Lenti pool. Flow analysis is performed to confirm the expression of CD8a and CD8b. Cells are stained with CD3, CD4, CD8A, and CD8B. As shown in FIG. 6, CD8A and CD8B double positive population is increased from 46.74% in the Jurkat NFAT_CD8Lenti pool to 95.74% in the #41 clone. This substantial increase of CD8 expression would allow us to capture better neoantigen reactive Class I TCRs. However, the CD4 expression was still not optimal.

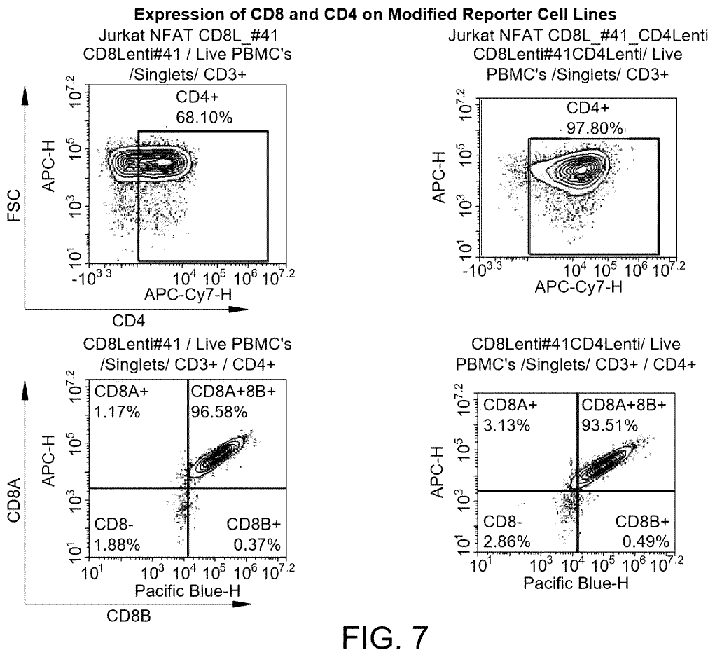

To improve the CD4 expression in Jurkat NFAT CD8Lenti #41, the cells are infected with lentivirus (pGenLenti-CD4_IRES_Puro). Flow analysis is then performed to evaluate the expression of CD4 by these cells. Cells are stained with CD3, CD4, CD8a, and CD8b. As shown in FIG. 7, the CD4 positive population is increased from 68% to 97.8%. CD8 expression is not changed significantly. Now upgraded #41 clone is both high CD8 and CD4 which improves TCR screening sensitivity.

### 2.2.2 Reporter Activity Time Course

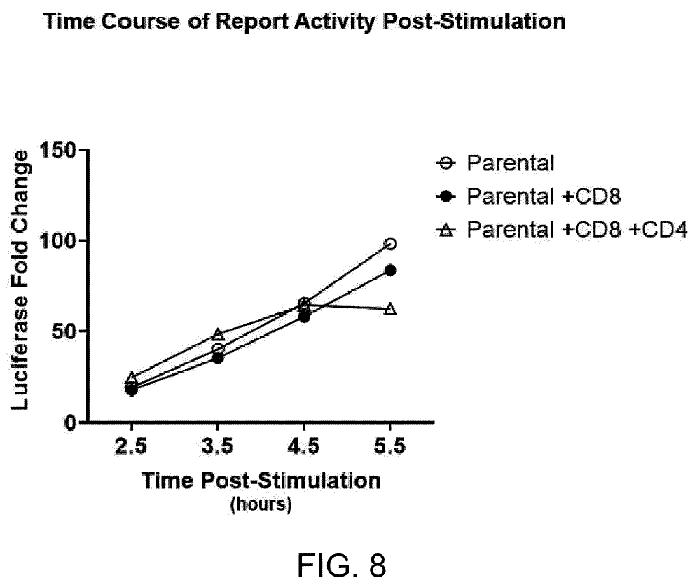

A time course study is performed to determine the best time point to harvest the co-culture. Jurkat cells are seeded in RPMI complete medium at 200 k cells/well in 96-multiwell plates. Cells are treated with 50 ng/ml PMA and 1 μg/ml Ionomycin for 2.5, 3.5, 4.5 and 5.5 hrs. Cells are harvested and lysed with passive lysis buffer (Promega) at room temperature for 15 minutes. 50 pls of cell lysis is mixed with 100 μl of luciferase substrate (Promega). Luciferase signal intensities are detected with Luminometer. Luciferase activity folds changes are calculated by dividing PMA/Ionomycin treated condition to vehicle control treated conditions. As shown in FIG. 5, 4-5 hours is the best time to harvest cells since luciferase signals start to drop for the CD8Lenti_CD4Lenti pool. Data is shown in FIG. 8.

### 2.3 Optimization of Transfection Conditions in COS-7 Cells

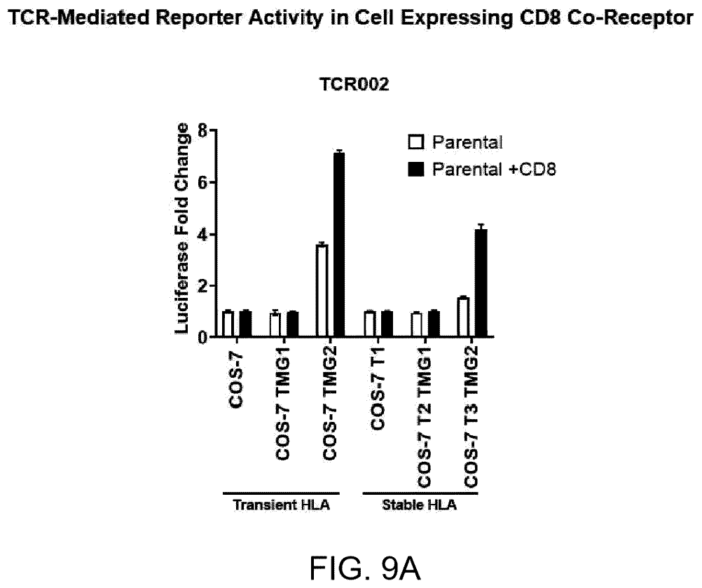

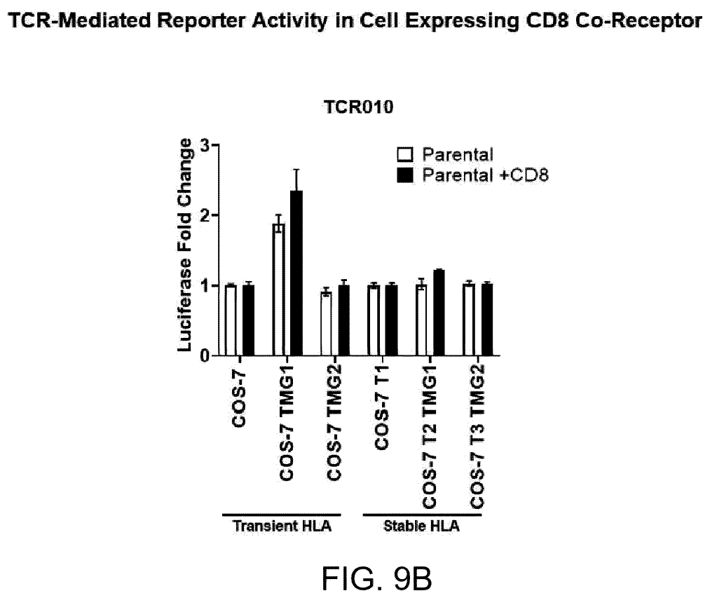

Day 1: COS-7 cells are seeded at 20,000 per well overnight in 96 multiwell plates. Day 2: COS-7 cells are transfected in each well with 150 ng of TMG1 or TMG2 and 75 ng of HLA A*11:01 and 75 ng of HLA A*02:01. Day 3: NEON transfection system is set up the following day and 5 million cells are electroporated with either TCR002 or TCR010 monkey-TCR (mTCR). Day 4: Jurkat cells are harvested and seeded on top of either transfected COS-7 cells or COS-7 cells stably expressing HLA A*11:01 or HLA A*02:01. After 5 hours co-culture, cells are harvested, and luciferase activity is measured. As shown in FIG. 9A, TCR002 TCR electroporated cells specifically recognized TMG2 (contained KRAS G12V mutation). As shown in FIG. 9B TCR010 TCR specifically recognized TMG-1 (contained R175H mutation) transfected COS-7 cells as expected. Transient transfection works better than stable pools in both TCR002 and TCR010 TCRs. In addition, Jurkat NFAT CD8Lenti has higher fold induction compared with Jurkat NFAT parental cells in both TCR co-culture experiments demonstrating the relevance of overexpressing CD8 in Jurkat NFAT cells for Class I TCRs.

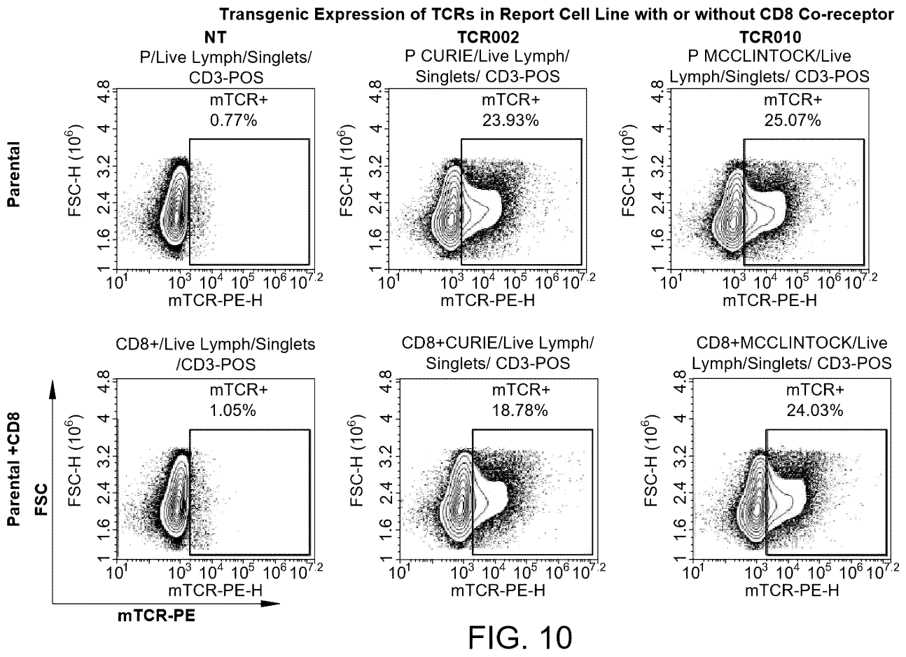

Jurkat NFAT electroporated with TCR002, TCR010 cells are analyzed using flowcytometry to detect the percentage of cells with mTCR expression. Cells are stained with CD3, CD4, CD8a, CD8b and mTCR antibodies. As shown in FIG. 10, cells express similar level of mTCR in Jurkat NFAT CD8Lenti cells compared with Jurkat NFAT parental cells. Over 90% of cells are viable in all six cell lines on the next day after electroporation suggesting the NEON electroporation system could provide highly viable T cells with sufficient percentage of mTCR expression (~20%). This would allow co-culture experiments to be performed the next day without wasting time to recover cells. CD8 co-receptor expression did not improve TCR expression therefore suggesting that the addition of CD8 improved the TCR-peptide:MHC interaction to improve the reporter activity.

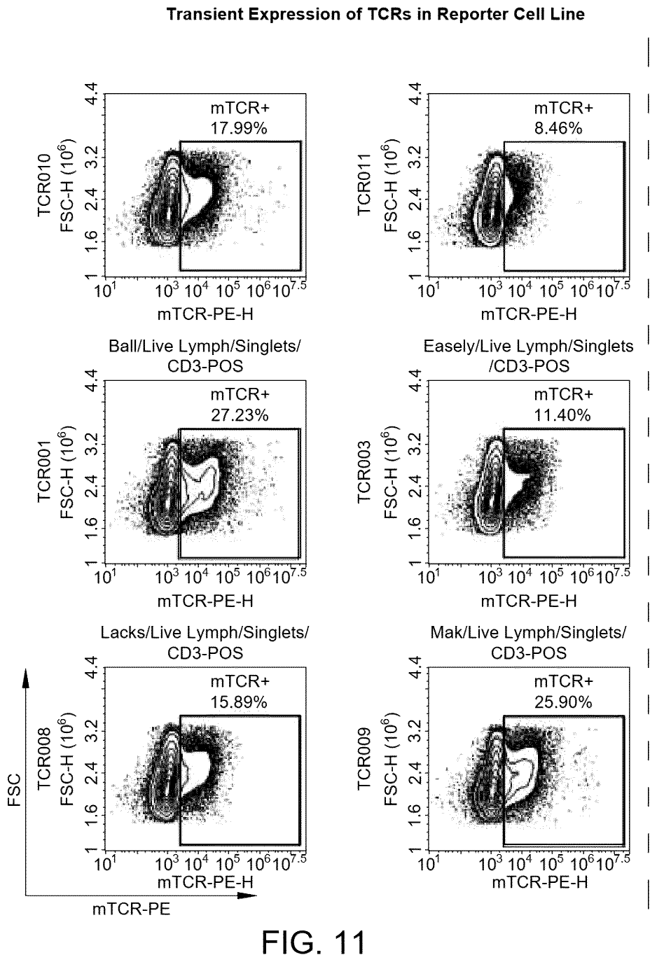

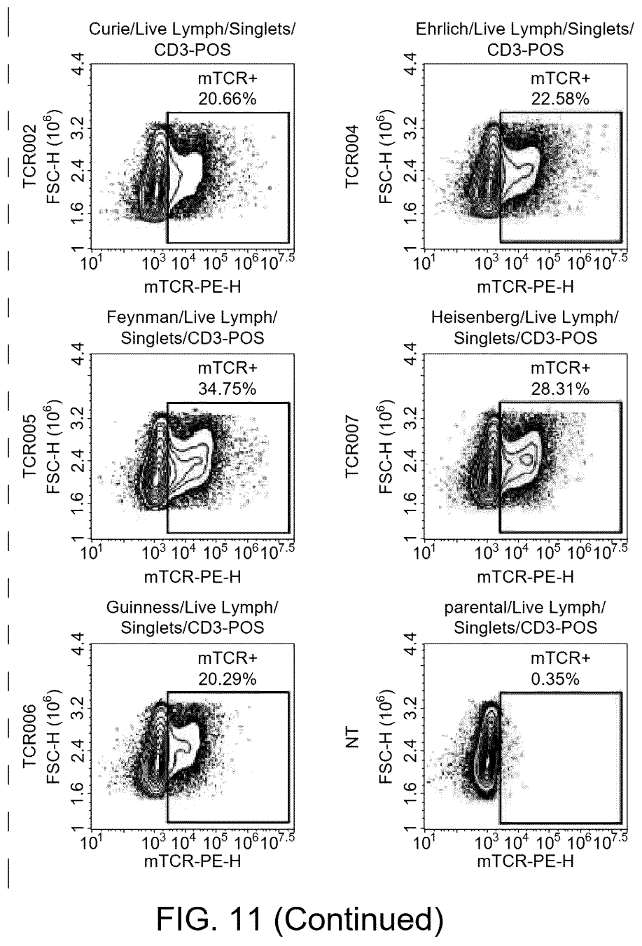

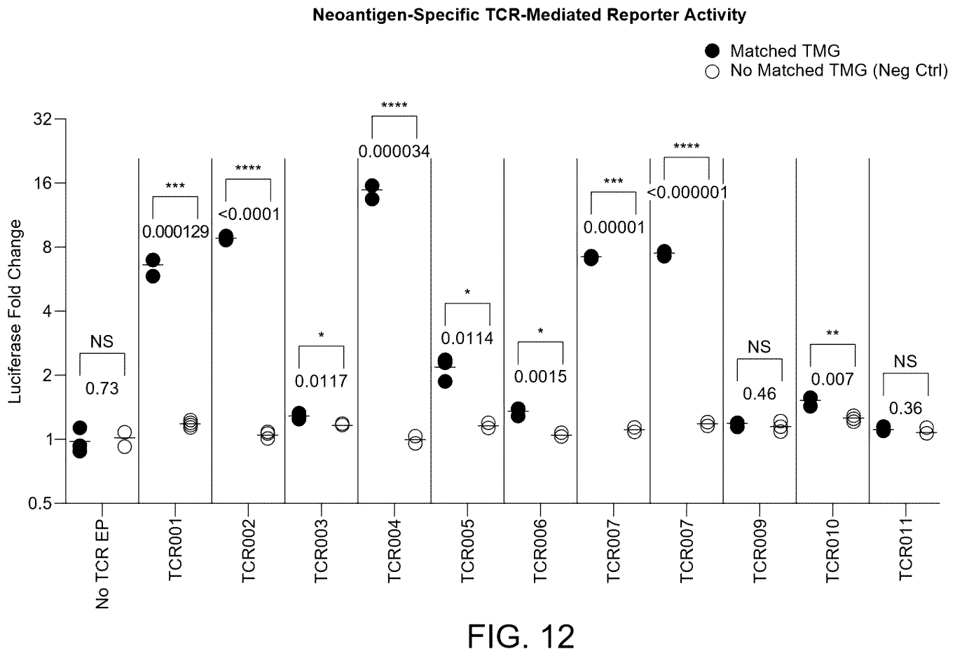

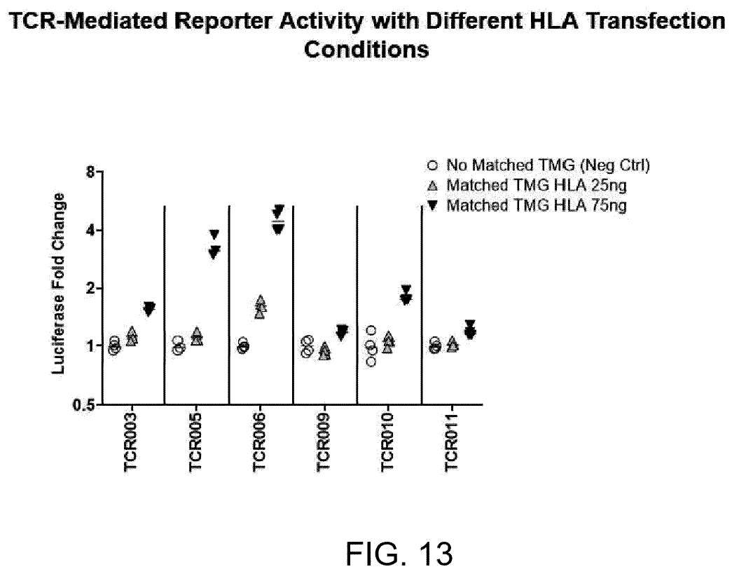

To examinate the reliability of JNR/COS co-culture system, several exemplary TCRs were tested. Flow analyses were performed to evaluate the mTCR expression level in 11 TCRs, and cells are stained with CD3, CD4, CD8a, CD8b and mTCR antibodies. As shown in FIG. 11, mTCR expression varied from 8-35% (9 of 11 TCRs expressed above 15%) when cells are gated on CD3+. Day 1: COS-7 cells are seeded at 20,000 per well overnight in 96 multiwells. Day 2: COS-7 cells are transfected in each well with 150 ng of TMGs and 150 ng of HLAs (each 25 ng). Day 3: NEON transfection system is set up the following day and 5 million cells are electroporated with each TCR plasmid. Day 4: Jurkat cells are harvested and seeded on top of transfected COS-7 cells. After 5 hours of co-culture, cells are harvested, and luciferase activity is measured. Luciferase activity fold change (FC) is calculated based on cells without electroporation using TCR. TCR specific HLAs and matched neoantigens or TMGs are listed on the table below. (FIG. 12) Based on the statistical analysis, 9 of 11 TCRs from TCR library are confirmed with specificity against matched TMGs (i.e., a match TMG contained mutations specific to the TCR). No matched TMGs are irrelevant TMGs where no specific mutations are contained in the plasmid to serve as negative control. To troubleshoot the TCRs with low reactivity experiments are designed by transfecting different amounts of HLA plasmids. As you could see from FIG. 13, the signal to noise ratio is significantly increased when COS-7 cells are transfected with 75 ng of plasmids compared with 25 ng. This has been observed in all 6 TCRs which show relatively low reactivity based on FIG. 12.

### 2.4 Optimization of Peptide Pulsing Conditions in COS-7 Cells

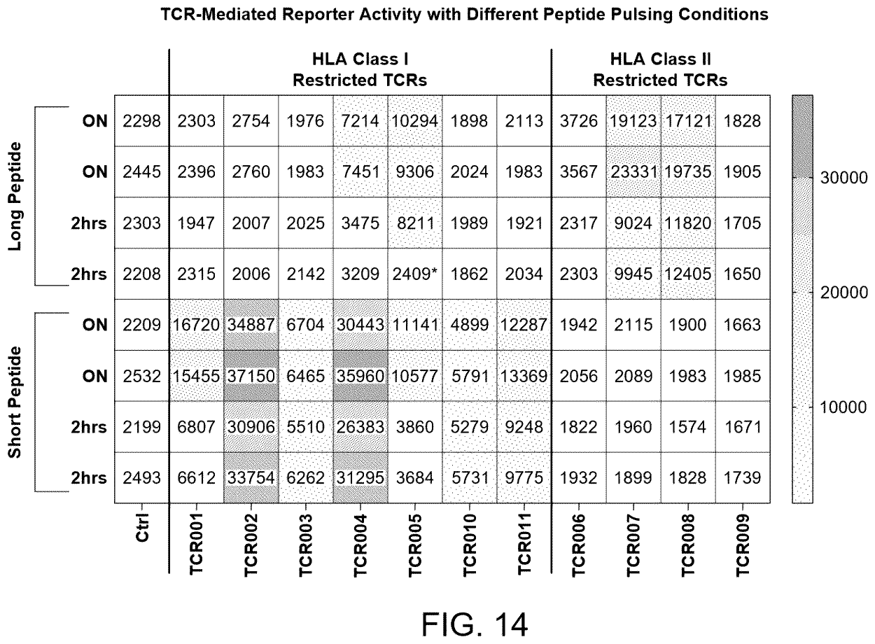

Peptide pulsing is tested with certain TCRs. COS-7 cells are pulsed with peptides either overnight or for 2 hours. Long peptides, as well as short peptides are used. 11 TCRs are electroporated for optimization studies. As shown in FIG. 14, three of 7 class I TCR are able to detect long peptide; however, all of the 7 class I TCRs are also able to react to short peptides. In addition, 3 of 4 Class II TCRs are reactive more to long peptides but not short peptides. In conclusion, overnight pulsing of peptide showed stronger signal compared with 2 hours. Class I TCRs recognize short peptide better and Class II TCRs recognize longer peptide better. The COS-7 peptide pulsing worked with most of the TCRs tested which demonstrates that COS-7 cells can be used to identify specific neoantigens in the reactive TMGs.

### 2.5 Development of Assay Controls

### 2.5.1 Anti-TCR Coated Plate Positive Control

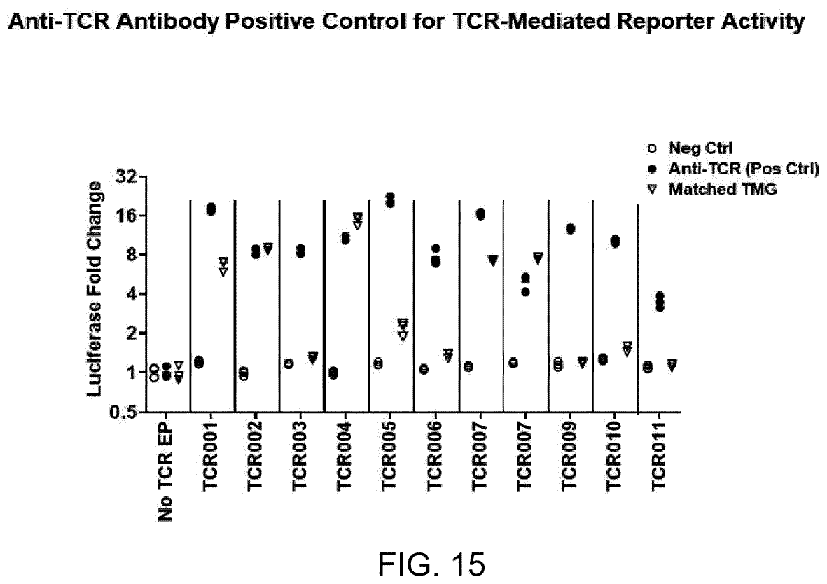

On the day of electroporation, H57 antibody is coated on the 96 multiwell plate overnight at 4° C. as a positive control. On the next day, Jurkat cells are seeded on the plate for 5 hours. Luciferase activity fold change (FC) is calculated based on cells without electroporation using TCR. Some of the TCRs demonstrated comparable levels of activation as H57 such as TCR002, TCR004, TCR001, TCR007 and TCR008 (FIG. 15). Some TCRs including TCR011, TCR009 and TCR006 are not activated as much with matched TMG they were with H57 coating suggesting that the TCR is successfully electroporated, but not fully activated. This might be due to the sub-optimal formation of HLA-neoantigen-TCR complex.

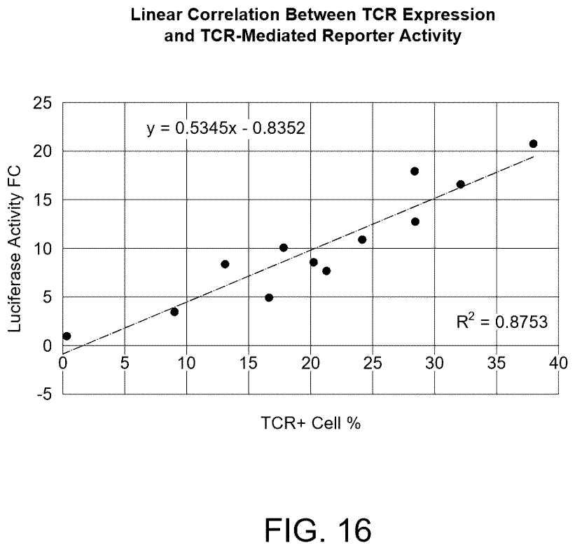

A scatter blot is generated using H57-coated Jurkat NFAT cells luciferase activity and mTCR expression based on the flow analysis. These cells are 12 cell lines shown in FIG. 16. Luciferase activity was positively associated with mTCR expression with R² value of 0.8753 suggesting that luciferase activity from H57 coated plate could serve as optimal control besides flowcytometry for TCR expression and biological function.

### 2.6 Conclusion

The series of data described in this example illustrate the development of a method and cell lines that are used to screen TCRs isolated from primary T cells against various combinations of HLA and antigens. Optimal reporter activity is observed between 4-5 hours after stimulation. It is observed that addition of CD4 and CD8 co-receptors to the reporter cells improved TCR-mediated reporter activity. Isolation of a single CD8-modified report cell line clone, Clone #41 is achieved which improved the sensitivity of the assay to detect reactive TCRs. Development of an assay positive control, using plate-bound anti-TCR antibody, proved to be a robust control for functional TCR expression and correlated highly with the frequency of TCR expression measured by flow cytometry. Modulation of HLA plasmid amounts in the transfection reaction is found to improve the antigen-presentation and subsequent sensitivity of detecting reactive TCRs in this assay. Overall, the example illustrates the development and optimization of a high-throughput TCR screening platform to enable identification of TCR sequences, antigen-specificity, and HLA-restriction which could be used to identify novel therapeutic TCRs derived from primary tissues.

### Example 3: Patient 0013 Til Screening

This example demonstrates the identification and isolation of EGFRL^{858R} reactive tumor infiltrating lymphocytes (TIL) from surgically resected patient tumor.

### 3.1 Ex Vivo Expansion of Tumor Infiltrating Lymphocytes (TIL)

### 3.1.1. Patient Demographics and Sample Collection

Patient 0013 is a 59-year-old non-small cell lung cancer patient. Following a standard-of-care surgical resection, multiple tumor specimens from the lung totaling approximately 8.25 cm² are obtained for the purposes of somatic mutation calling, HLA typing, expanding TIL and screening TIL for neoantigen reactivity. Approximately 5 mL of whole blood is also collected to provide the patient's normal reference exome sequence to determine somatic mutations present in the tumor.

### 3.1.2. Tumor Sample Processing and TIL Expansion

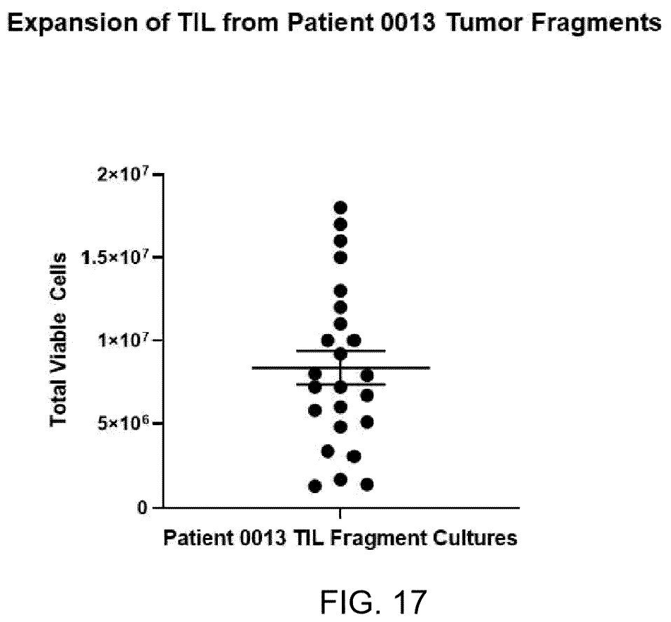

TILs are generated from the resected lung tumor specimen. Two fragments of tumor are snap frozen for mutation analysis. The remaining tumor is then divided into 24 fragments and each fragment is separated into 2-3 pieces. One piece from each group is selected and placed in the 24 wells tissue culture plate. The fragments are maintained in 2 mL of IL-2 50:50 media (6000 IU/mL) and cells are fed with 6000 IU/mL of IL-2 in 50:50 media two times per week. 50:50 culture media used for TIL culture is prepared by mixing of complete RPMI 1640 media (supplemented with 10% human serum and antibiotics) and AIM-V media at 1:1 ratio. After 4 weeks culture, TILs are counted and frozen at the density of 5×10⁶ cells/mL. A range of 1.3×10⁶ to 1.8×10⁷ viable TIL are obtained at the end of the four-week TIL expansion (FIG. 17).

### 3.1.3. Molecular Profiling Sample Processing

DNA is isolated from each of a frozen tumor sample and from a matched PBMC sample. Quantification by fluorescence spectrometry indicates that yields are sufficient for downstream applications, and gel electrophoresis demonstrated an absence of degradation in the isolated genomic DNA.

To assess somatic mutations, 200 ng tumor and 200 ng normal DNA are each processed through whole exome sequencing library preparation by way of hybrid capture. The final paired end libraries are sequenced on an Illumina NextSeqDx sequencer. Libraries are sequenced at 2×101 bp read lengths. The sequencing run yielded sufficient read number and quality for subsequent analysis. Reads are subject to on-board demultiplexing to yield paired FASTQ files.

### 3.1.4. Molecular Profiling Analysis Pipeline

Raw reads from WES experiments are aligned to the human hg19 reference genome using BWA to create BAM files. Duplicate reads (paired reads mapped to identical locations of the genome) are discarded to avoid enrichment bias from PCR overamplification. To improve mapping quality, indel realignment and base quality recalibration were performed on BAM files.

Somatic mutation calling is performed on the tumor WES data using the normal WES data as the reference sequence. The mutation detection algorithms Mutect, MuSE, Varscan2, Mutect2, and Strelka are used to detect SNVs, the latter three are used to detect indels, and Mutect2 is used to detect MNVs. An SNV is only reported if it is detected by at least three of the five algorithms. An indel is reported if it is detected by at least one algorithm.

The detected mutations are annotated with ANNOVA and VEP. Only mutations meeting the following criteria are included in the final report: allele frequency (AF) >0.05 in the tumor sample; coverage at that position of at least 20 reads in the tumor sample and 10 reads in the normal sample; normal sample AF<0.02. The resulting mutations are filtered further to include SNVs and indels that are deemed to be non-synonymous to generate a final list of mutations.

Potential neoantigens are predicted for each mutation. An in silico strand representing a mutant peptide of up to 25 amino acid residues are derived, given that antigen lengths in human cells range from 8 to 25 bases. For every non-synonymous SNV, MNV, and in-frame indel, the in silico strand sequence is initiated 12 amino acid residues upstream of the mutated residue and end 12 amino acid residues downstream of the mutated residue. For frameshift indels that result in a variant more than one residue in length, amino acids are included in the in silico strand until a stop codon is detected in the new reading frame. If multiple transcripts are known to overlap the somatic mutation position, an in silico strand is derived for each such transcript and all unique strands are reported for each somatic mutation.

Class I and II HLA alleles are derived from WES data. Optitype, Polysolver and HLAVBSeq are applied to infer class I HLA alleles at two-field/four-digit resolution (e.g., HLA-A*02:01). HLAVBSeq is applied to infer class II HLAs. The in silico strand peptide sequences and the HLA types are input together to netMHCpan4.1 to predict potential interactions.

### 3.1.5. Molecular Profiling Results

WES analysis reveals 92 unique somatic non-synonymous mutations ranging in allele frequencies from 0.051 to 0.521. The somatic mutations produced 92 unique in silico neoantigen candidates.

### 3.2. Design and Construction of Synthetic Reagents

### 3.2.1 Neoantigen Reagent Design

To create peptide fragments containing the patient's somatic mutations, a total of 92 in silico strands representing non-synonymous mutations are synthesized as peptides with crude quality. To create vectors containing the same somatic mutations in nucleic acid form, the 92 amino acid sequences are reverse translated in silico and codon optimized for expression in human cells. A total of 6 tandem minigenes (TMGs) are designed by concatenating a set of 15 such amino acid sequences into one open reading frame. Incidental BamHI, EcoRI, NotI and NheI sites are removed by replacing codons within the restriction sites with synonymous codons. The nucleotide sequence GAG AAT TCG (codes for Glu (E)/Asn (N)/Ser (S) and contains an EcoRI restriction site) is added to the 5′ end of each TMG gene, and the nucleotide sequence AAG GAT CCC (codes for Lys (K)/Asp (D)/Pro (P) and has a BamHI restriction site) is added to the 3′ end of each TMG gene.

### 3.2.2 TMG Plasmid Synthesis

Each TMG, together with the flanking restriction sites, is synthesized and cloned into the masterTMG_pcDNA3.1(+) plasmid in frame with existing start and stop codons using EcoRI (5′) and BamHI (3′) restriction enzymes. Each final plasmid is prepared in TE with 95%±5% supercoiled plasmid and ≤0.005 EU/μg endotoxin content.

### 3.2.3 Human Leukocyte Antigen (HLA) Plasmid Assembly

Peptide sequences for each HLA allele found in the patient sample are retrieved from the IPD-IMGT/HLA Database (ebi.ac.uk). Each peptide sequence is reverse translated in silico and codon optimized for expression in human cells. A BamHI restriction site and a Kozak site are added at the 5′ end of the coding sequence and an EcoRI restriction site and stop codon are appended to the 3′ end. The assembled sequence is synthesized and cloned into pcDNA3.1(+) using BamHI and EcoRI restriction enzymes. The resulting plasmids are prepared in TE with 95%±5% supercoiled plasmid and ≤0.005 EU/μg endotoxin content.

### 3.3. Screening of Ex Vivo Expanded TIL

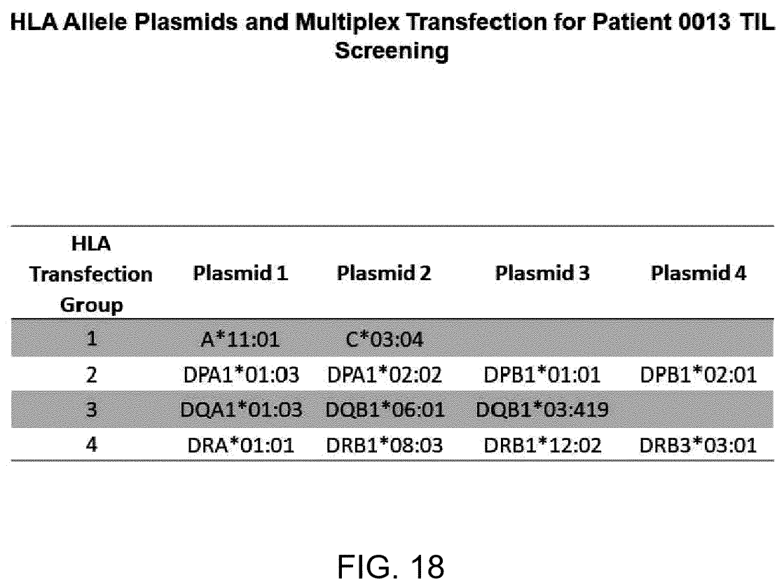

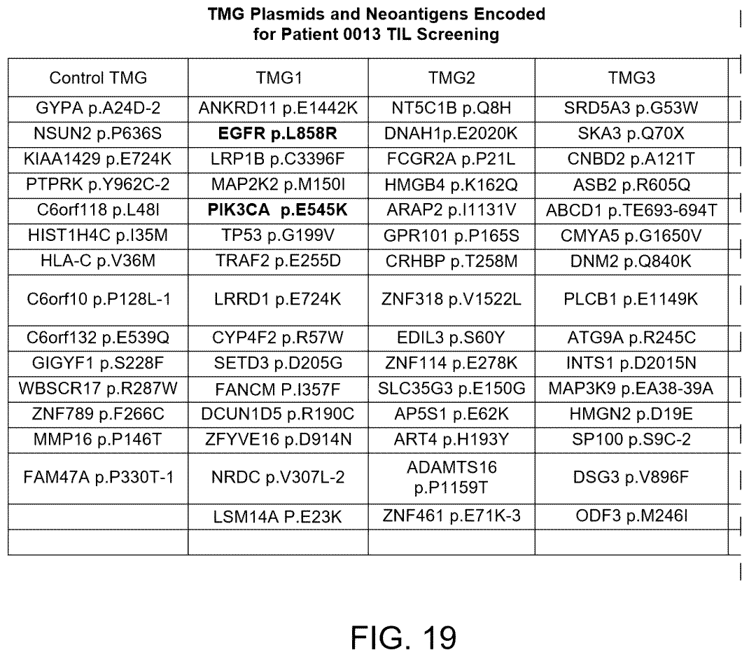

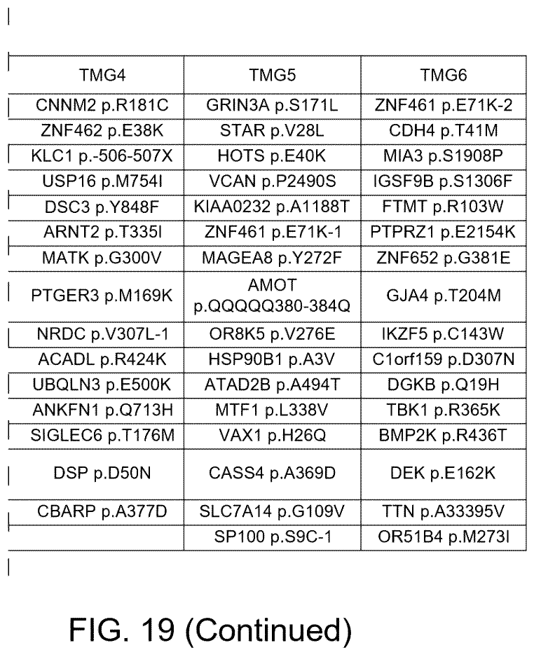

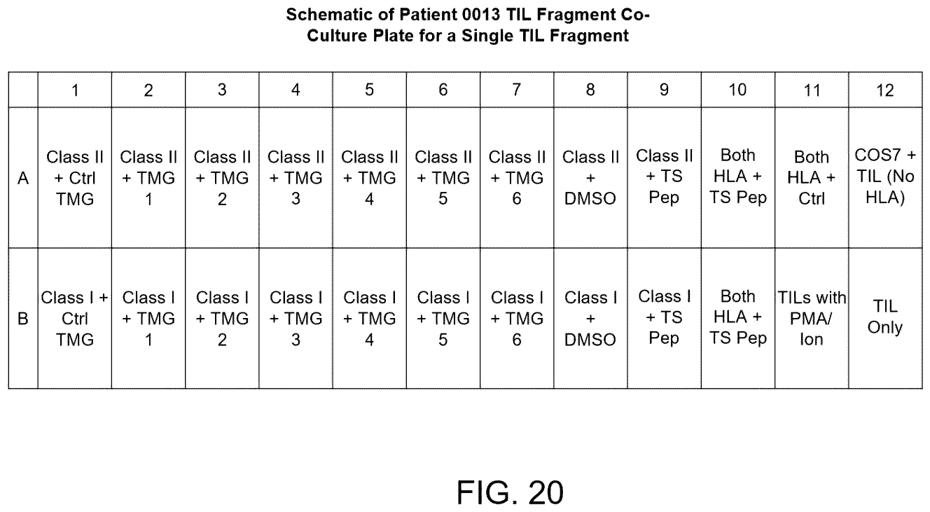

On day 1, neoAPCs are seeded in 96 wells at 20,000 cells per well and incubated overnight at 37° C. TILs are thawed and recovered with IL-2 at 3000 IU/μL. On day 2, the cell medium is replaced with antibiotic-free DMEM medium prior to transfection. A total of 150 ng of TMG plasmid and 300 ng (total) HLA plasmids are transfected using Lipofectamine 2000. To enhance screening throughput, 2-4 HLA plasmids are transfected together in one well (FIG. 18). Additional conditions are included in which all the HLA Class I and HLA Class II plasmids are transfected together. The transfection mix, containing HLA and TMG plasmids (FIG. 19) is added to each well and cells are cultured overnight at 37° C. On day 3, the medium on the neoAPCs is replaced with fresh medium containing peptide pools. Peptides are prepared at 50 mg/mL and pulsed at a final concentration of 10 μg/mL. Peptides are pooled together to mirror their grouping within each TMG. Only HLA Class II plasmid transfected cells were pulsed with peptide pools. As negative controls, irrelevant TMG plasmid and peptides are used. To specifically identify the reactivity of TILs to the neoantigens produced by the frequent mutations in tumor patients (TopSpot, TS) peptides: EGFR p.L858R and PI3KCA p.E545K), neoAPCs cells were transfected with HLA I, HLA II or both HLAs and incubated with 10 g/mL of these two peptides sequentially (TS pep). Additional negative controls include TILs incubated with neoAPCs cells without transfection. ELISpot plates are coated with anti-interferon gamma capture antibody (1-DIK) overnight. On day 4, ELISpot plates are washed with PBS and blocked with complete RPMI media (10% FBS) for 1 hour. NeoAPCs are harvested using trypsin. TILs are counted and resuspended at a concentration of 2×105 cells/mL. Medium is poured out from each ELISpot plate. 50 μL of medium, 100 μL of neoAPCs, and 100 μL of TILs (20,000 cells) are added sequentially to each well of the ELISpot plates. Plates are transferred to a 37° C. incubator with 5% CO2 and incubated for 18-24 hours. An example of the plate layout for the co-culture of TIL from a single fragment is shown in FIG. 20. On day 5, cells of each well in the ELISpot plate is mixed by pipetting, then 200 μL of cells are carefully transferred from the ELISpot plate to a new 96-well U-bottom plate. These cells are later stained using a cocktail of CD3, CD4, CD8, and 4-1BB antibodies for phenotyping with flow cytometry. ELISpot plates are washed and incubated with an anti-IFN-γ antibody (Biotinylated 7-B6-1 biotin). Plates are incubated at RT for 2 hours in the dark. Plates are washed and incubated with Streptavidin-ALP at RT for 1 hour in the dark. Plates are washed with PBS and stained with BCIP/NBT substrate solution. Plates are incubated at RT for 15 minutes until distinct spots appear. Tap water is used to wash the plates gently but extensively, then the plates are left out until completely dry. Plates are analyzed using an ELISpot reader.

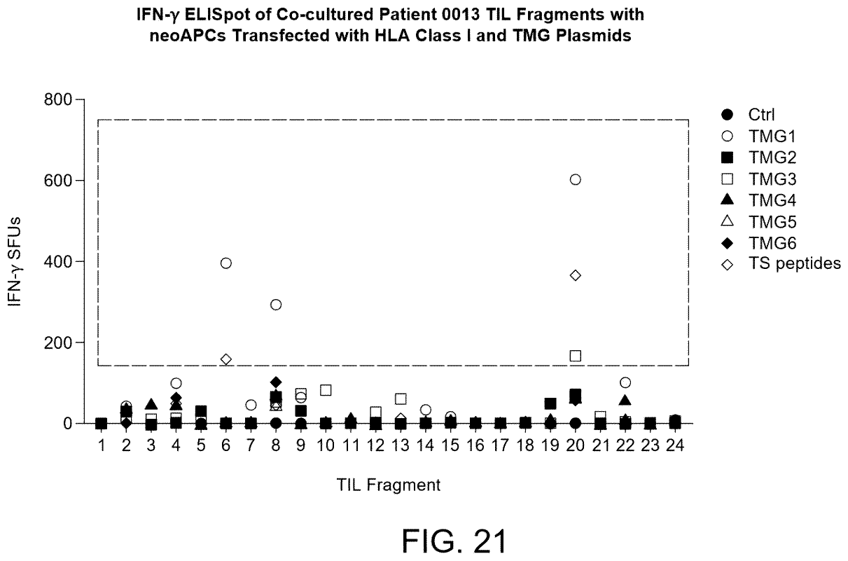

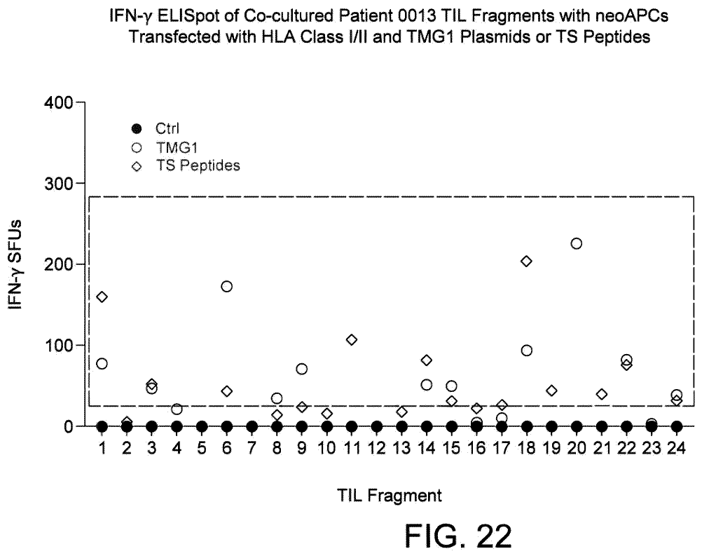

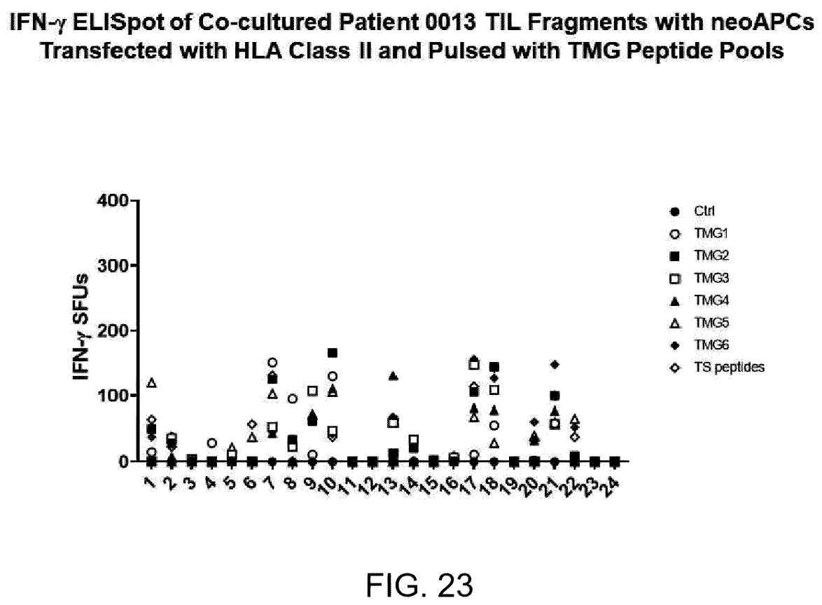

As shown in FIG. 21, wells containing neoAPCs transfected with HLA Class I plasmids and either TMG1 or TS peptides (both containing EGFR p.L858R and PIK3CA p.E545K) has higher numbers of spot forming units (SFUs) compared to other TMGs in TIL fragments 6 and 20. TIL Fragment 8, demonstrated a higher level of SFUs to TMG1 only. Similarly, neoAPCs expressing both HLA Class I and Class II alleles demonstrated reactivity to either TMG1 (TIL fragments 8, 9, 20) or TS peptides (TIL fragments 11, 19, 21) or both (TIL fragments 1, 3, 6, 14, 15, 18, 22, and 24) by an increase in IFN-γ SFUs (FIG. 22). In contrast, there were no specific reactivities observed in any of the HLA Class II plasmid conditions with the 24 TIL fragments (FIG. 23).

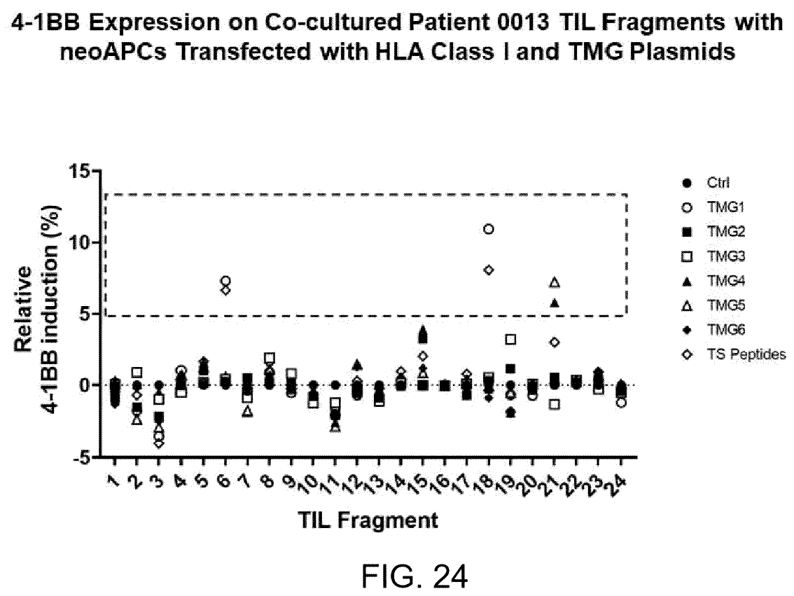

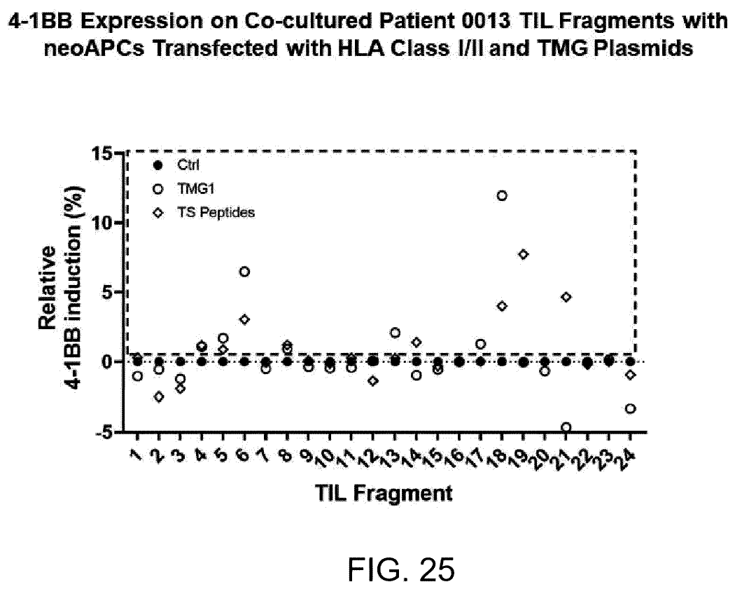

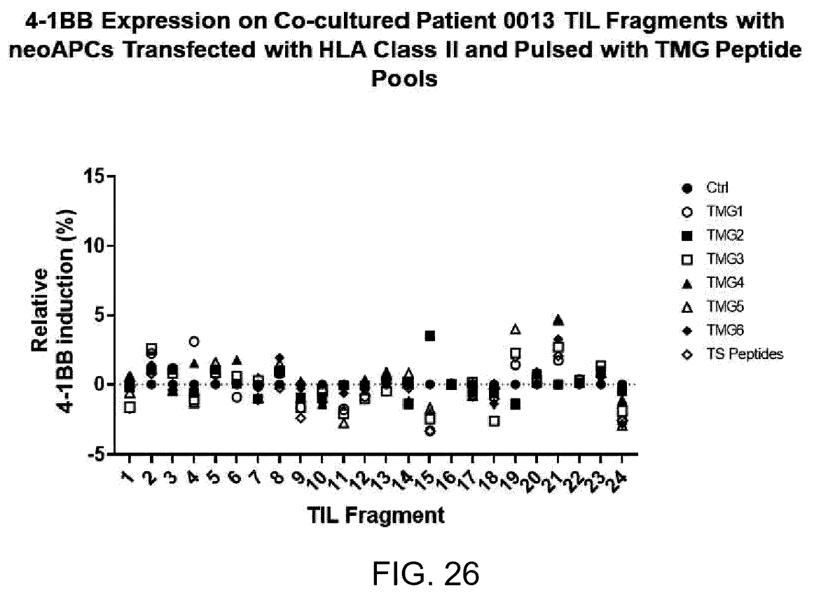

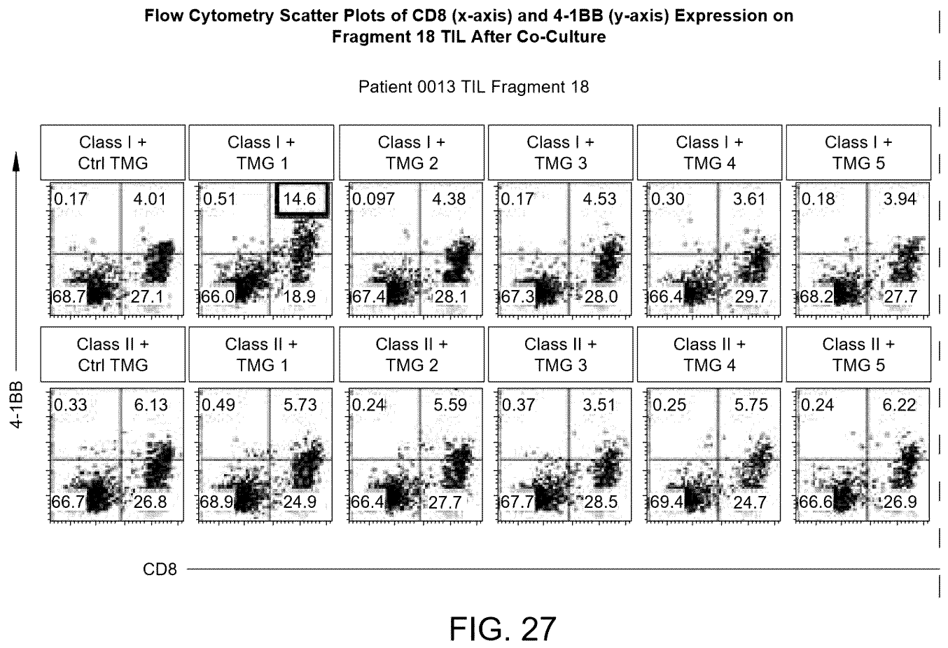

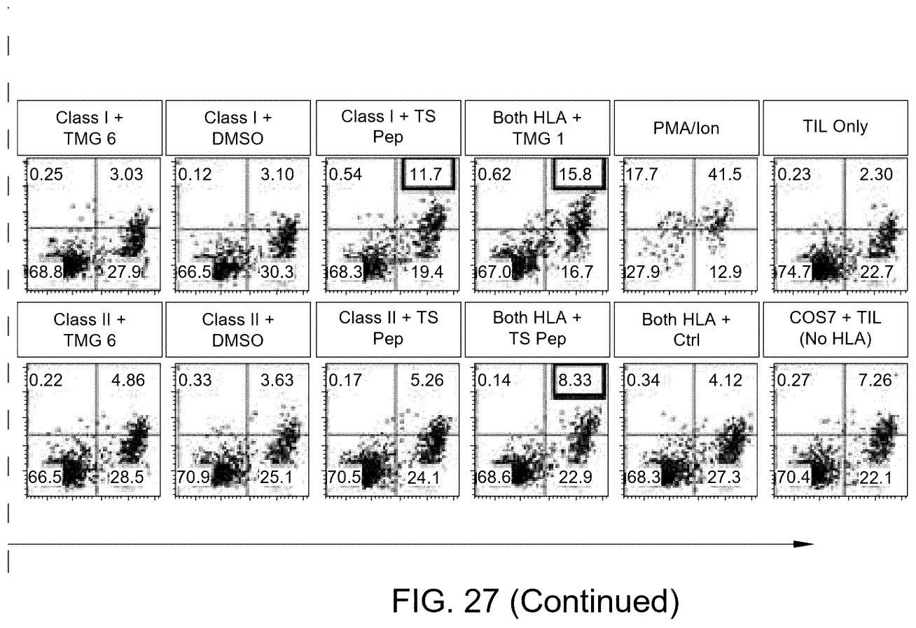

In addition to secretion of IFN-7, co-cultured TILs were harvested from the ELISpot plates and stained using a cocktail of CD3, CD4, CD8, and 4-1BB antibodies for phenotyping with flow cytometry. Induction of 4-1BB expression above background indicates T cell activation. In TIL co-cultures with HLA Class I transfected neoAPCs, increases in 4-1BB were observed against TMG1 and TS Peptides (TIL fragment 6 and 18), and TMG 4 and TMG5 (TIL fragment 21) (FIG. 24). The latter reactivity was not mirrored in the IFN-γ ELISpot results. Similarly, TILs co-cultured with both HLA Class I and Class II alleles demonstrated upregulation of 4-1BB to either TMG1 (TIL fragments 13 and 17), TS Peptides (TIL fragments 14, 19 and 20) or both TS Peptides and TMG 1 (TIL fragments 4, 5, 6, 8 and 18) (FIG. 25). In agreement with the IFN-γ ELISpot results, TIL co-cultured with HLA Class II neoAPCs did not demonstrate specific upregulation of 4-1BB (FIG. 26). Representative scatter plots of 4-1BB expression of all conditions tested for TIL fragment 18 shown in FIG. 27 illustrate that the T cell activation is specifically observed in the CD8+ TIL population.

Taken together, the results of the TIL fragment screening suggested that there is specific reactivity to either EGFR p.L858R or PIK3CA p.E545K. Furthermore, the reactivity is restricted to either HLA-A*11:01, HLA-C*03:04. This reactivity was observed broadly across a number TIL fragments, including fragments 6, 8, 14, and 18.

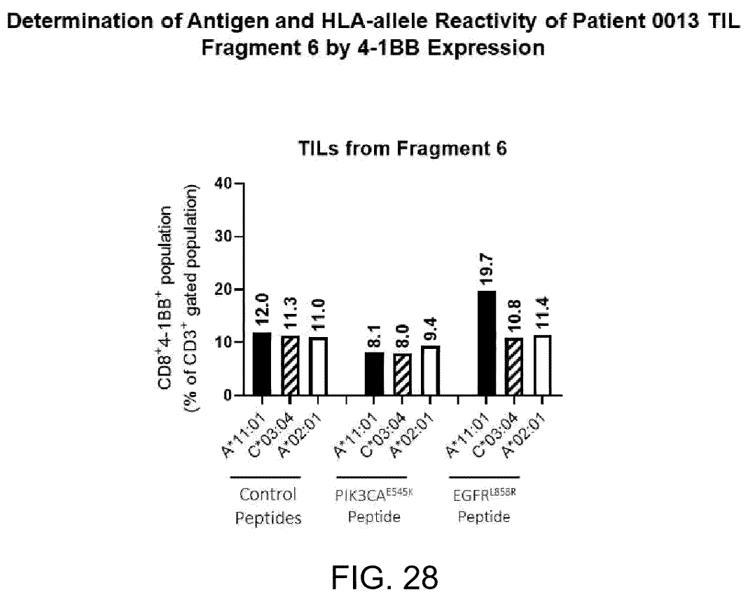

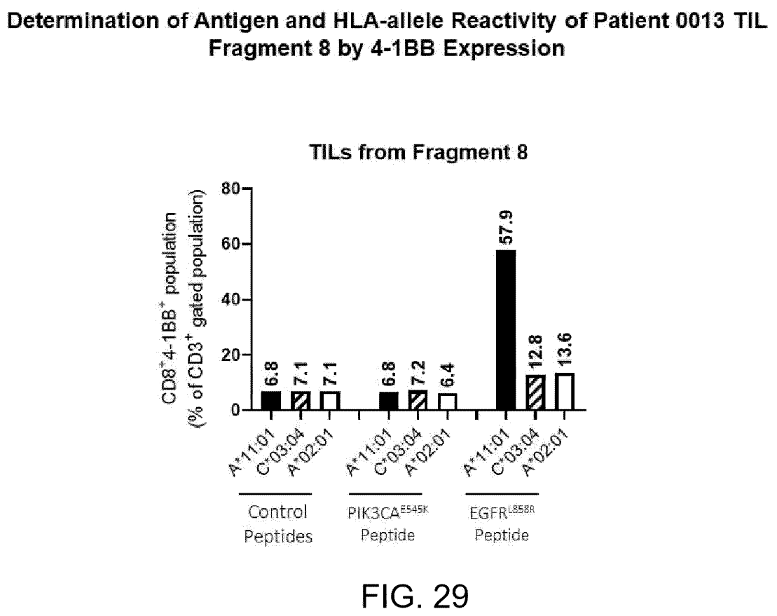

To determine the specific neoantigen/HLA reactivity of the TIL a parsing process is performed. NeoAPCs are transfected with plasmids encoding HLA A*11:01, HLA C*03:04 or HLA A*02:01 and pulsed with control peptides, PI3KCA p.E545K. or EGFR p.L858R. Then, 4-1BB assay was carried out with TILs from Fragment 6 and 8 to identify the activating HLA/neoantigen condition as described above. TIL fragments 6 and 8 are chosen for parsing due to limited cell numbers in other fragments. The 4-1BB results showing induction of 4-1BB in the TILs cocultured with EGFR p.L858R peptide pulsed, HLA A*11:01 expressing neoAPCs (FIG. 28 and FIG. 29). The data illustrates that TILs from fragment 6 and 8 are specifically reactive to EGFR p.L858R neoantigen presented in the context of HLA-A*11:01.

3.4. Isolation of Neoantigen Reactive TCR Sequences from Ex Vivo Expanded TIL

### 3.4.1 TIL Co-Culture for Single Cell RNA Sequencing

Co-cultures with TIL pooled from fragments 6, 8, 14, and 18 are prepared as described above with neoAPCs co-transfected with HLA-A*11:01 and pulsed with the EGFR L858R MUT 25mer peptide (Table 7). Co-cultures are incubated at 37° C. for four hours before being harvested. Cells are then stained with an antibody cocktail containing fluorescently labeled antibodies against CD45, CD3, CD4, CD8, 4-1BB, and a Live/Dead dye. Stained cells are sorted for live CD3+ cells using a Sony SH800 sorter. Single cell suspensions are prepared from the sorted cells and used as input for single cell RNA sequencing.

3.4.2 Single cell RNAseq of Sorted TIL

To sequence TCRs, sorted TIL are processed through the Chromium Next GEM Single Cell 5′ Reagent Kit v2 from 10× Genomics targeting 10,000 cells. The resulting cDNA is processed through the Chromium Single Cell Human TCR Amplification Kit VDJ per manufacturer's recommendations.

Molarity of final libraries is determined using the size for fragments between 100 and 1000 bp on the Agilent TapeStation 4150 (Agilent High Sensitivity D1000 ScreenTape assay) and library concentration from Qubit 4 (Life Technologies Qubit dsDNA BR Assay kit). Libraries are pooled with a 1% PhiX spike-in. The library pool is clustered and sequenced at 26+96 on an Illumina NextSeqDx 550 using a 150 cycle High Output kit for a target coverage of 5000 reads per cell for VDJ and 20,000 reads per gene expression library. Raw bcl files are yielded.

### 3.4.3 Analysis of the 10× Gene Expression (GEX) and VDJ Sequencing Data

The GEX and VDJ sequencing data are preprocessed using the CellRanger toolkit (version 7.0) provided by 10× Genomics. The BCL files from the Illumina sequencer are converted to raw FASTQ files. The FASTQ files for the GEX and VDJ experiments are processed separately. GEX reads are aligned to the human GRCh38 reference genome. Cell barcodes assignment and UMI counting are then performed to create a single-cell gene expression matrix. Doublets and cells with >10% mitochondria gene counts are filtered out in the study. Then the raw read counts are normalized and scaled using Seurat. About 2,000 highly variable genes are identified using the FindVariableGenes module. Next, principal component analysis (PCA) and uniform manifold approximation and projection (UMAP) are performed for dimension reduction and a shared nearest neighbor (SNN) algorithm is applied to cluster the cells.

The raw VDJ reads are assembled into contigs using a graph-based algorithm with the aid of the pre-built reference sequence from the IMGT database. Cells with identical productive V(D)J transcripts are placed into a same clonotype.

### 3.4.4 Integrating the 10× GEX and VDJ Data

For each TCR clonotype, the corresponding cells in V(D)J are projected to the identified clusters in the GEX data. The full-length FASTA sequences of both the TRA and TRB chains, as well as the amino acid sequences of the CDR3 regions for each clonotype are also reported.

3.4.5 Identification of Potential Neoantigen Reactive TCR Clonotypes from Single-Cell RNAseq

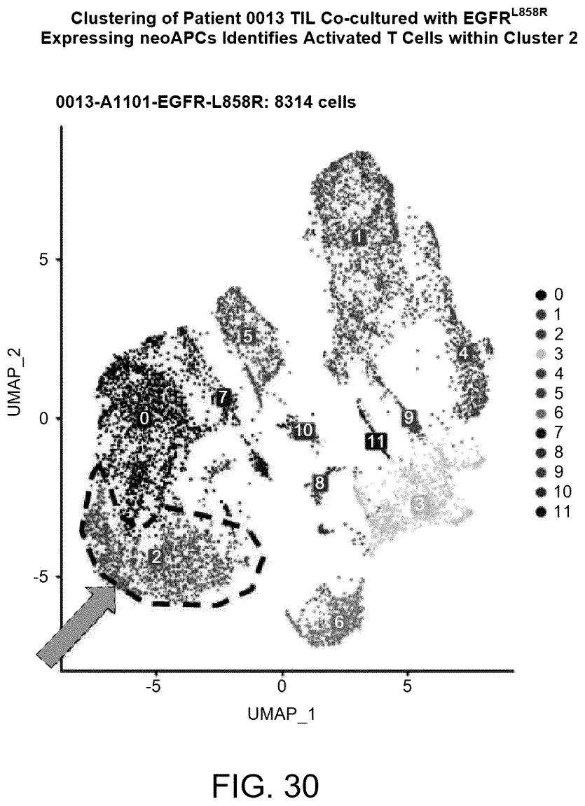

Plotly (4.10.1) interactive maps are generated from Uniform Manifold Approximation and Projection (UMAP) coordinates, Shared Nearest Neighbor (SNN) clusters, gene expression data, and clonotype information generated from Seurat (4.2.1) in R (4.2.1). This output is used to inform TCR selection (FIG. 30).

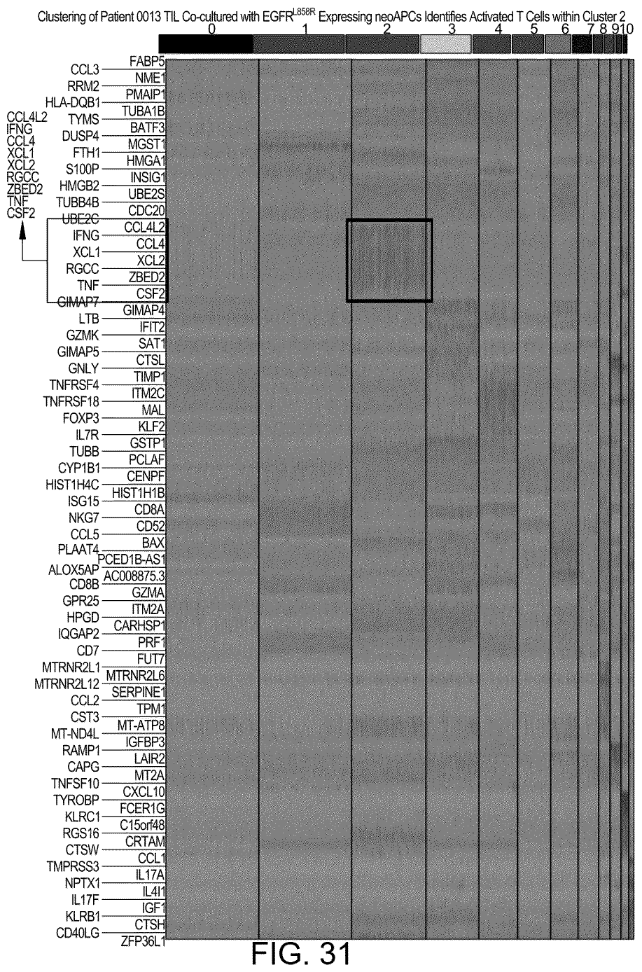

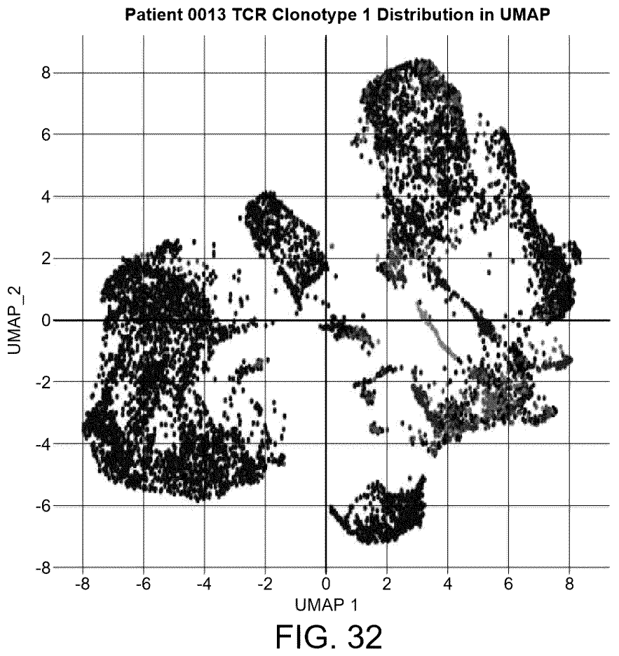

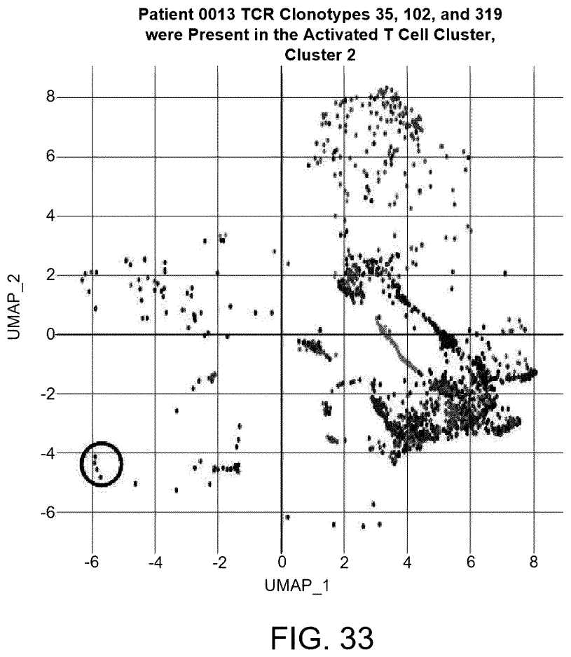

For Patient sample 0013 TIL, clonotype 1 is the dominant population. Using the standardized Plotly outputs, Seurat cluster 2 shows upregulation of T-cell activation associated genes CCL4L2, IFNG, CCL4, XCL1, XCL2, TNF, CSF2 (FIG. 31). This activated cluster is used to select potentially neoantigen reactive HLA-A*11:01 restricted TCRs against the EGFR p. L858R neoantigen. Clonotype 1 of the CD8+ T-cells is selected as it is the major population (FIG. 32). The Plotly output is then used to select three additional minor clonotypes (C35, C102, C319) (FIG. 33), all of which has substantial upregulation of the neoantigen reactive TIL signature (“XCL2”, “XCL1”, “IL2”, “CSF2”, “IFNG”, “CCL4”, “CCL4L2”, “TNF”, “CCL3”, “RGCC”, “TNFSF9”, “DUSP2”, “NFKBID”, “MIR155HG”, “NR4A3”, “EVI2A”, “CRTAM”, “ZBED2”, “FABP5”, “PIM3”, “NR4A1”, “IL10”, “TNFSF14”, “NR4A2”, “LINC00892”, “ZFP36L1”, “GZMB”, “MYC”, “SPRY1”, “KDM6B”, “EGR2”, “PHLDA1”, “PPP1R2”, “VSIR”, “REL”, “PRDX1”, “SLA”, “CYTOR”, “DDX21”, “IER3”, “PGAM1”, “NAMPT”, “HSP90AB1”, “IL23A”, “FAM107B”, “BCL2A1”, “ZEB2”, “ZBTB32”, “BTG2”, “GADD45B′”, “RILPL2”, “SEMA7A”, “TGIF1”, “SRGN”, “RAN”, “CFLAR”, “MAT2A”, “SIAH2”, “PRNP”, “RNF19A”, “FASLG”, “NME1”, “EVI2B”, “HSPH1”, “NOP16”, “CSRNP1”, “TAGAP”). Based on these findings, full length TCR sequences are reconstructed in silico and TCR expression plasmids are generated for subsequent functional testing.

### 3.4.6 TCR Reconstruction and Assembly

The three TCR clonotypes identified above (C1, C35, C102, C319) contained exactly one beta chain and one alpha chain sequence. Each raw beta chain sequence is modified by replacing all sequence 5′ of the start of the V region with an NheI restriction site, and the entire constant region is replaced with a BspI restriction site. Each alpha chain was modified by the replacement of all sequence 5′ of the start of the V region with an XmaI restriction site, and the constant region is replaced with a SaclI restriction site.

Rare codons (defined as codons used <10% according to the Homo sapiens codon usage table) are replaced with more frequently used synonymous codons throughout the beta and alpha gene open reading frames. Incidental NheI, BspI, XmaI, and SaclI restriction sites are eliminated from the open reading frame by replacing bases within the restriction sites with synonymous codons not found within each restriction site.

### 3.4.7 TCR Plasmid Assembly

Each alpha and beta gene is synthesized independently and subcloned into the pZT2 expression vector using the synthesized restriction sites (NheI and BspEI for the beta gene and XmaI and SaclI for the alpha gene). Each final plasmid is prepared in 10 mMTris-HCl, pH 8.0, 1 mM EDTA (TE) with 95%±5% supercoiled plasmid and ≤0.005 EU/μg endotoxin content.

3.5. In Vitro Screening of TCR Candidates by hunTR®
3.5.1 Co-Culture of TCR Reporter Cells with neoAPCs

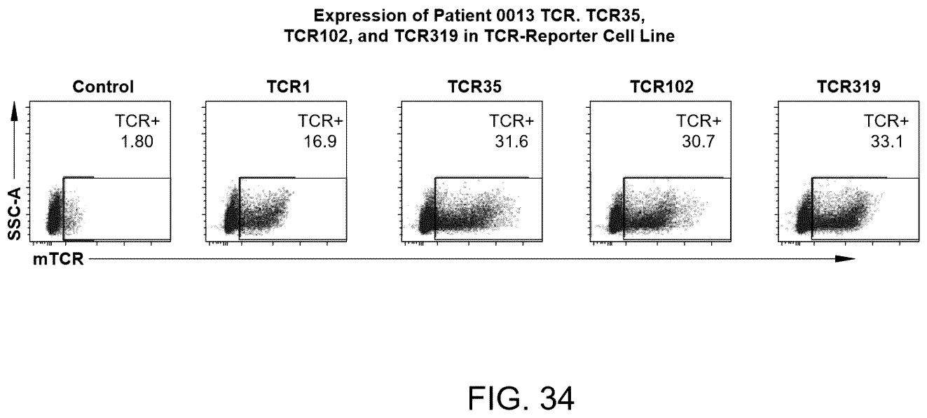

On day 1 neoAPCs are seeded at 20,000 per well (96 multiwells) overnight (in 37° C. incubator). The following day cell medium is replaced with antibiotic free DMEM medium before transfection. The Master tandem minigene (MasterTMG) and HLA plasmids are transfected using lipofectamine 2000. TCR Reporter cells are counted and seeded at 0.5 million/ml with fresh RPMI1640 complete medium overnight to enhance electroporation efficacy (10% FBS and 1% Pen/Strep). Using the NEON transfection system, TCR Reporter cells are electroporated with pZT2 vectors encoding the full length, paired-chain TCR1, TCR35, TCR102, and TCR319. Electroporated cells are transferred immediately in 24 multiwell culture plate containing antibiotic free RPMI medium and incubated overnight to recover. The transgenic TCR expression is measured with flow cytometry using antibody cocktail CD3, CD4, CD8A, CD8B and H57-597. All four TCRs evaluated, TCR1, TCR35, TCR102, and TCR319 expressed in the TCR Reporter cells with expression ranging from 16.9% to 33.1% (FIG. 34).

EGFR wild type p.L858 and mutant p.L858R peptides were designed based on the HLA-A*11:01 bindings predictions to the EGFR L858R MUT 25mer sequence. The top predicted mutant epitopes and corresponding wild type sequences are listed in Table 7. Peptides are prepared at 50 mg/ml and pulsed at 1 ug/ml onto HLA-A*11:01 transfected neoAPCs. After overnight peptide pulsing, neoAPCs are washed and co-cultured with TCR Reporter cells. After 4-5 hours incubation, cells are harvested from the wells and induction of the luciferase reporter is quantified BioTek reader.

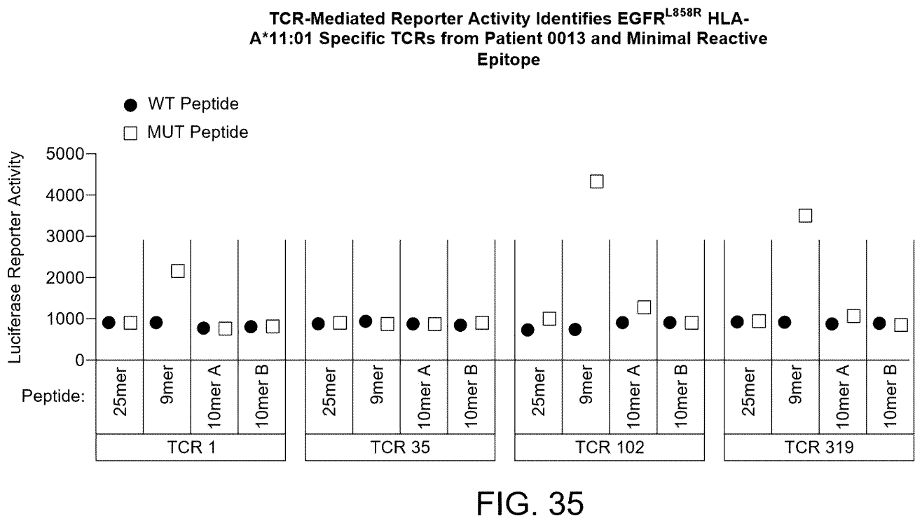

Reporter activity results indicated that TCR1, TCR102, and TCR319 demonstrated EGFR p.L858R neoantigen-specific recognition (FIG. 35). In contrast, TCR35 did not demonstrate any reporter activity suggesting this TCR clonotype is not likely specific for the EGFR p.L858R HLA-A*11:01-restricted neoantigen. Importantly, TCR1, TCR102, and TCR319 demonstrated specific recognition of EGFR p.L858R mutant peptides and not wild type EGFR peptides. Interestingly, the reactivity against the different EGFR p.L858R peptides suggests that the EGFR L858R MUT 9mer peptide is the minimal epitope recognized by TCR1, TCR102, and TCR319. Moreover, TCR102 and TCR319 demonstrated some recognition of EGFR L858R MUT 10mer A, but not EGFR L858R MUT 10mer B. These two peptides contain the same core sequence but differ in the N- and C-terminal amino acids. These data suggest that the exposed C-terminal lysine (K) is important for HLA binding.

TABLE 7
EGFR Peptides Tested

| Peptide | Sequence^{a} | SEQ ID NO: |
|---|---|---|
| EGFR L858 WT 25 mer | KTPQHVKITDFGLAKLLGAEEKEYH | 293 |
| EGFR L858R MUT 25 mer | KTPQHVKITDFGRAKLLGAEEKEYH | 294 |
| EGFR L858 WT 9 mer | KITDFGLAK | 295 |
| EGFR L858R MUT 9 mer | KITDFGRAK | 296 |
| EGFR L858 WT 10 mer A | VKITDFGLAK | 297 |
| EGFR L858R MUT 10 mer A | VKITDFGRAK | 298 |
| EGFR L858 WT 10 mer B | KITDFGLAKL | 299 |
| EGFR L858R MUT 10 mer B | KITDFGRAKL | 300 |

^{a}Variant amino acid bolded and underlined

### 3.6. Validation of TCRs in Primary Human T Cells

Experiments are conducted to verify that the identified TCRs are expressed and function to specifically recognize the EGFR L858R HLA-A*11:01 neoantigen to activate primary human T cells.

### 3.6.1 Generating EGFR-L858R-A-1101 TCR-T Cells

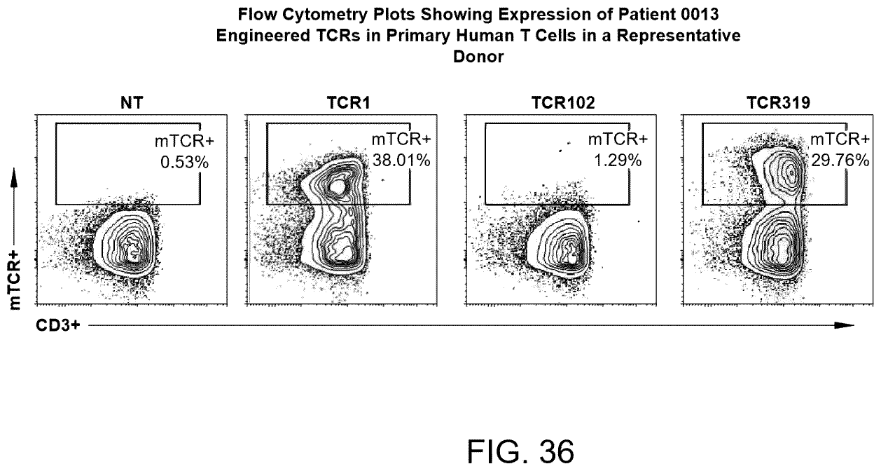

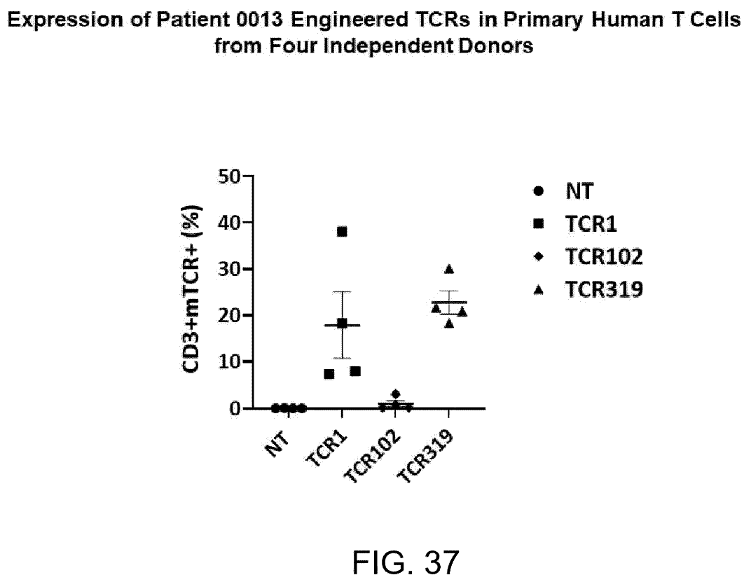

PBMC cells are thawed, spun down, resuspended in electroporation buffer together with TCR plasmid (Sleeping Beauty transposon) and SB11 (Sleeping Beauty transposase), and electroporated. Following electroporation, cell suspensions are collected, transferred to recovery media and incubated in a 37° C./5% CO2 incubator overnight. Within 24 hours post-electroporation (Day 1), live cells are transferred to G-REX® culture plates and incubated with first expansion media (50:50 media containing IL-2+IL-21+ T Cell TransAct™). Cells are fed regularly with cytokines. After 11 days of first phase expansion, Transgenic mTCR+ cells are isolated with anti-mTCR (H57-597) antibody. The isolated mTCR+T cells are transferred to G-REX® culture plates and incubated with a second expansion media (50:50 media containing IL-2+ T Cell TransAct™) Cells are fed regularly with cytokines. After 14 days of second phase expansion, cells are harvested, phenotyped by flow cytometry to determine the mTCR expression, and evaluated in functional assays to determine the TCR specificity and function. Representative contour plots from one donor are shown in FIG. 36. Interestingly, TCR102 expression is not detectable in the harvested TCR-T cells. The expression of TCR1, TCR102 and TCR319 is summarized in FIG. 37 and demonstrates that TCR1 and TCR319 are effectively expressed in primary human T cells using Sleeping Beauty transposon/transposase system of gene transfer.

### 3.6.2 Generation of Monocyte-Derived Dendritic Cells (DCs)

The DCs are derived from peripheral blood monocytes by adherence method and differentiation in vitro. Briefly, cryopreserved PBMCs from an HLA-A*11:01 positive donor are thawed, washed, resuspended in AIM-V media (Invitrogen, Carlsbad, CA) and seeded in a T175 flask. Non-adherent PBMCs are removed and the remaining adherent cells are cultured in RPMI-1640 media containing 5% human AB serum and supplemented with 800 IU/mL GM-CSF and 200 IU/mL IL-4 to differentiate and maintain the monocyte-derived dendritic cells. Cells are cultured for a total of 6-9 days, with addition of cytokine-supplemented media every 2-3 days. At the end of the culture, both the non-adherent and adherent cell fractions were harvested and phenotyped for DC markers.

3.6.3 Coculture of TCR-T Cells with Peptide-Pulsed Dendritic Cells

For the co-culture assay, DCs are resuspended in 50:50 media and pulsed with neoantigenic peptide specific to the TCR target. The concentration of peptide used is titrated to exponentially decreasing concentrations (10, 1, 0.1, 0.01, and 0.001 g/mL) to establish a dose-dependent effect of EGFR p.L858R target neoantigen peptide on TCR signaling as a surrogate of TCR binding avidity. After the incubation, peptide-pulsed DCs are seeded at 2.5×104 cells/well in a round-bottom 96 well plate. TCR-T cells are added to the peptide-pulsed DCs at 1×105 cells/well. DCs pulsed with only DMSO served as a background control, while DCs alone or T cells alone serve as negative controls. DCs pulsed with EGFR L858 wild type (wt) peptide serves as an additional control to demonstrate the specificity of the TCR target. For positive controls, T cells alone are stimulated with a Cell Activation Cocktail (Biolegend, San Diego, CA) containing an optimized concentration of phorbol 12-myristate-13-acetate (PMA) and ionomycin. The DC-TCR-T cell co-culture is incubated for 18-24 h.

3.6.4 Measurement of T cell Activation by 4-1BB Expression

At the end of the DC-TCR-T cell co-culture, cells are stained with anti-4-1BB antibody to assess for TCR-specific T-cell activation and co-stained with antibodies against mTCRP (clone H57-597), CD3, CD4 and CD8 for T cell phenotyping. Cells are acquired and analyzed on the flow cytometer (Novocyte Quanteon, Agilent Technologies, Santa Clara, CA), while data analysis is performed using NovoExpress software.

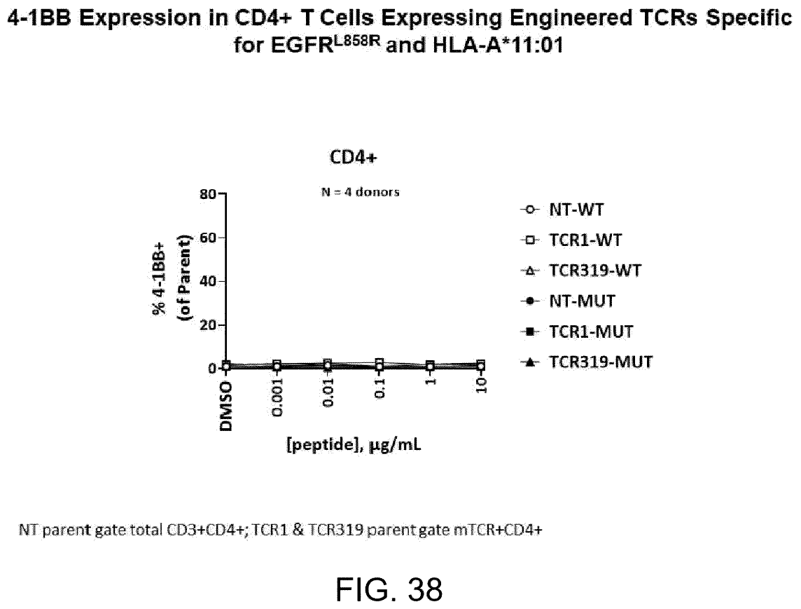

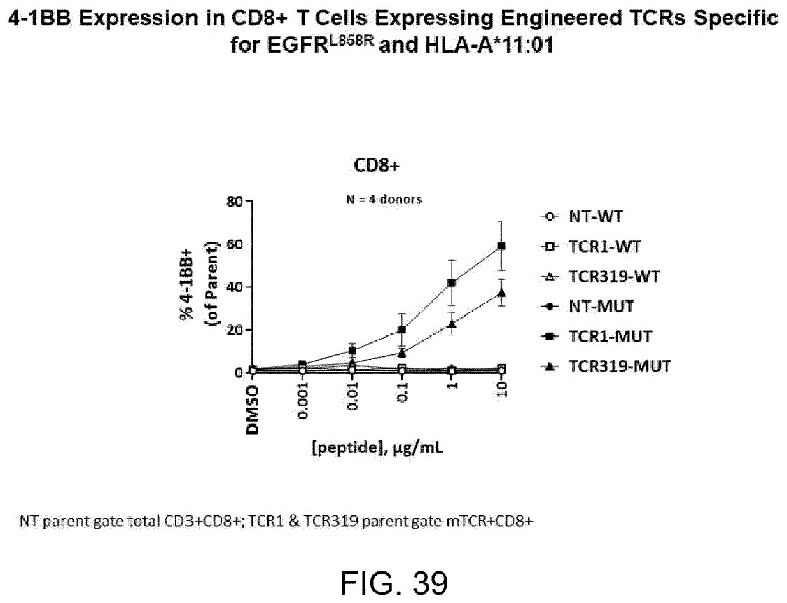

Upregulation of 4-1BB following DC co-culture was evaluated in both the mTCR+CD4 and CD8 T cell populations. Within the CD4+mTCR+ population, no induction of 4-1BB is observed (FIG. 38) at any concentration of EGFR L858R peptide. In contrast, 4-1BB upregulation is observed in the CD8+mTCR+ cell population for both TCR1 and TCR319 (FIG. 39) suggesting that both TCR1 and TCR319 have CD8-coreceptor dependent recognition of the pHLA complex. Importantly, both TCR1 and TCR319 demonstrate specific recognition of the EGFR L858R mutant peptide and not the EGFR L858 wild type peptide.

### 3.6.5 TCR-T Cell Cytoxicity Against Tumor Cells

NT T cells and TCR-T cells are cocultured with HLA*A11:01-transfected Saos2 tumor cells at an effector:target (E:T) ratio of 4:1 in 96-well white high-binding Optiplates (PerkinElmer, Waltham, MA) for 18-24 hours. Tumor cells are pulsed with 1 g/mL wt or mutant EGFR peptides for 2 hours at 37° C. Subsequently, peptides are washed, and T cells are added to initiate the co-culture, which is incubated overnight at 37° C.

After overnight incubation, the viability of adherent tumor cell targets is assessed by the CellTiter-Glo 2.0 Cell Viability Assay (Promega, Madison, WI), an ATP-based assay for detection of viable cells, after removal of cells in suspension (e.g., T cells and detached tumor cells). Luminescence was measured and recorded using a luminescence plate reader, the BioTek Cytation 1 (BioTek, Winooski, VT). Percent target cell lysis was calculated by taking the luminescence from remaining adherent viable tumor cells relative to controls. Peptide-loaded tumor cells and TCR-T cells individually served as controls for the maximum signal/counts and minimum signal/counts, respectively.

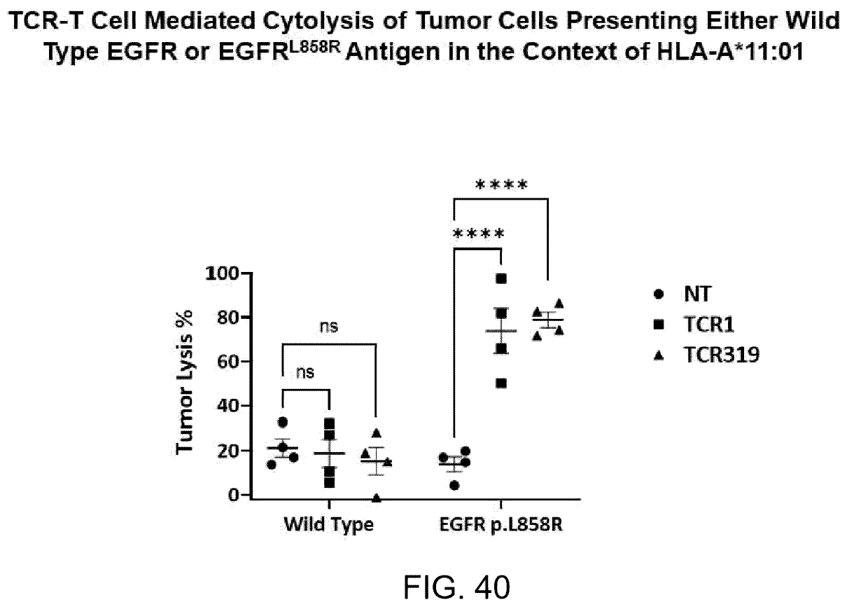

Tumor targets with the EGFR L858R neoantigen are specifically lysed by TCR-T cells expressing TCR1 or TCR319, but not NT T cells (FIG. 40). Cell killing of wild type EGFR L858 peptide-pulsed tumor cells by TCR1 and TCR319 TCR-T cells is comparable to the NT control T cells and demonstrates that TCR1 and TCR319 do not exhibit significant killing of non-target cells (FIG. 40). Collectively, these data demonstrate that TCR1 and TCR319 can be stably expressed in primary human T cells and are capable to redirecting T cell activation and killing to EGFR L858R HLA-A*11:01 presenting target cells.

A variety of further modifications and improvements in and to the compositions, systems, and methods of the present disclosure will be apparent to those skilled in the art. The following non-limiting embodiments are specifically envisioned:
1. A method for identifying a neoantigen-reactive T cell receptor (TCR), comprising:
   i) co-culturing
   a) a reporter T cell comprising a TCR expression cassette, and
   b) an antigen presenting cell (APC) that expresses a target neoantigen sequence and a matched human leukocyte antigen (HLA) sequence; and
ii) identifying a positive reporter signal in the reporter T cell to identify a neoantigen-reactive TCR.
   2. A method for identifying a neoantigen-reactive TCR, comprising:
i. obtaining single-cell gene expression profiles from a population of tumor infiltrating lymphocytes (TIL) isolated from a patient sample,
ii. performing bioinformatics analyses on the single cell gene expression data to identify TCR clonotypes of interests,
iii. creating recombinant TCR sequences;
iv. preparing a reporter T cell comprising a TCR expression cassette encoding a TCR sequence reconstructed from paired TCR α and β chain sequences identified from the clonotype of interest in step ii,
v. preparing a tandem minigene (TMG) expression vector;
vi. analyzing the patient sequencing data to identify class I and class II HLA alleles and preparing HLA expression vectors comprising the class I HLA and class II HLA allele sequences;
vii. preparing an antigen presenting cell (APC) comprising transfecting the TMG expression vector and one or more HLA expression vectors into a cell wherein each transfection condition comprises a TMG and one or two HLA types;
viii. co-culturing the reporter T cell in step iii with the APC of step vii,
ix. identifying a positive reporter activity in the reporter T cell to identify a neoantigen-reactive TCR.

3. The method of embodiment 2, further comprising obtaining whole exome sequence (WES) data from the patient sample and analyzing the WES data to identify class I and class II HLA alleles.

4. The method of embodiment 2, wherein the performing bioinformatics analysis further comprises clustering the TCR clonotypes and to select a clonotype of interest.

5. The method of embodiment 2, wherein the creating recombinant TCR sequences comprises designing alpha and beta TCR sequences in silico.

6. The method of embodiment 2, wherein the TMG comprises nucleic acid sequences for the expression of concatenated amino acid sequences of non-synonymous single nucleotide variants (SNVs).

7. The method of embodiment 2, wherein the clustering comprises grouping the TCT clonotypes by CD8 or CD4 expression, gene function of differentially expressed genes, and the level of expression of each TCR.

8. The method of embodiment 2, further comprising culturing the reporter T cell having the identified neoantigen-reactive TCR with one or more peptides representing the non-synonymous single nucleotide variants (SNVs) present in the TMG.

9. The method of embodiment 1, wherein the APC is a COS-7 cell.

10. The method of embodiment 1 or 2, wherein the reporter T cell is an immortalized T cell line.

11. The method of embodiment 10, wherein the immortalized T cell is a Jurkat cell.

12. The method of embodiment 11, wherein the Jurkat cell is a Jurkat NFAT cell.

13. The method of any one of embodiments 1 to 12, wherein the TCR expression cassette comprises a TCR sequence reconstructed from TCR α and β chain sequences identified from tumor infiltrating lymphocytes (TILs) isolated from a tumor sample, and wherein the target neoantigen sequence and the matched HLA sequence are identified from the same tumor sample.

14. The method of any one of embodiments 1 to 13, wherein the method comprises identifying TCR sequences from TILs isolated from a tumor sample.

15. The method of embodiment 7, wherein the method further comprises identifying somatic mutations in the tumor sample and determining the germline HLA typing of the tumor sample.

16. A method of identifying a neoantigen-reactive T cell receptor (TCR), comprising:
i) obtaining TCR α and β chain sequences from tumor infiltrating lymphocytes (TILs) isolated from a patient sample;
ii) obtaining neoantigen sequences comprising somatic mutations present in the tumor sample, and the germline HLA typing of the patient sample;
iii) co-culturing
   a) a reporter T cell expressing a TCR sequence reconstructed from the TCR α and 3 chain sequences obtained in step i), and
   b) an antigen presenting cell (APC) that expresses a neoantigen sequence and a matched human leukocyte antigen (HLA) sequence obtained in step ii); and
iv) evaluating the reporter activity in the reporter T cell to identify a neoantigen-reactive TCR.

17. The method of embodiment 16, wherein the APC is a COS-7 cell, and the reporter T cell is a Jurkat NFAT cell.

18. The method of any one of embodiments 13 to 17, wherein the isolated TILs are first expanded ex vivo and then co-cultured with APCs modified to express relevant HLA alleles and antigens obtained from the tumor sample.

19. The method of any one of embodiments 1 to 18, wherein the reporter T cell comprises a reporter system that is activated by the binding of the TCR to the neoantigen.

20. The method of any one of embodiments 1 to 19, wherein the reporter T cell and the APC are co-cultured in a ratio of 1:16, 1:8, 1:4, 1:2, 1:1, 2:1, 4:1, 8:1, or 16:1.

21. The method of any one of embodiments 1 to 20, wherein the reporter T cell and the APC are co-cultured for 1-48 hours.

22. A TCR, or an antigen-binding portion thereof, isolated according to the method of any one of embodiments 1 to 21.

23. A TCR, or an antigen-binding portion thereof, comprising a sequence selected from the group consisting of SEQ ID NOs: 1-36 (the sequences provided in Tables 1-3).

24. A neoantigen/HLA complex, wherein the neoantigen comprises a sequence selected from the group consisting of SEQ ID NOs: 67 to 292 and wherein the HLA comprises a sequence selected from a group consisting of SEQ ID NOs: 58 to 66.

25. The neoantigen/HLA complex of embodiment 24, wherein the neoantigen is SEQ ID NO: 238 and the HLA sequence is SEQ ID NOs: 60 and 62 or SEQ ID NOs: 61 and 62.

26. The neoantigen/HLA complex of embodiment 24, wherein the neoantigen is SEQ ID NO: 142 and the HLA sequence is SEQ ID NO: 58.

27. The neoantigen/HLA complex of embodiment 24, wherein the neoantigen is SEQ ID NO: 151 and the HLA sequence is SEQ ID NOs: 65 and 63.

28. The neoantigen/HLA complex of embodiment 24, wherein the neoantigen is SEQ ID NO: 162 and the HLA sequence is SEQ ID NO: 59.

29. A recombinant vector expressing the T cell receptor, or an antigen-binding portion thereof, of embodiment 22 or 23.

30. A polynucleotide encoding an amino acid sequence that is at least 90%, 95%, 96%, 97%, 98%, 99%, or 100% identical to an amino acid sequence selected from the group consisting of SEQ ID NOs: 1-36 (the sequences provided in Tables 1-3).

31. A population of cells that comprise the recombinant vector of embodiment 29, or the polynucleotide of embodiment 30.

32. The population of cells of embodiment 31, wherein the recombinant vector or the polynucleotide is integrated into the genome of the population of cells.

33. The population of cells of embodiment 31 or 32, wherein the cells are immune effector cells.

34. The population of cells of embodiment 33, wherein the immune effector cells are selected from the group consisting of T cells, natural killer (NK) cells, B cells, mast cells, and myeloid-derived phagocytes.

35. A pharmaceutical composition comprising the population of cells of any one of embodiments 31 to 34, and a pharmaceutically acceptable carrier.

36. A method of preparing a medicament for the treatment or prevention of a medical condition, the method comprising preparing the population of cells any one of embodiments 31 to 34.

37. A method of treating a disease or medical condition, the method comprising administering the pharmaceutical composition of embodiment 23 to a patient in need.

38. The method of embodiment 36 or 37, wherein the disease or medical condition is a cancer.

39. A co-culture reporter system for identifying a neoantigen-reactive T cell receptor (TCR), comprising:
i) a reporter T cell comprising a TCR expression cassette, co-cultured with
ii) an antigen presenting cell (APC) that expresses a target neoantigen sequence and a matched human leukocyte antigen (HLA) sequence.

40. The co-culture reporter system of embodiment 39, wherein the APC is a COS-7 cell, and the reporter T cell is a Jurkat NFAT cell.

41. The method of embodiments 1 to 38, or the reporter system of embodiment 39 or 40, wherein the target neoantigen is expressed in an antigen encoding plasmid.

42. The method or the reporter system of embodiment 41, wherein the antigen encoding plasmid is a Tandem Minigene (TMG) plasmid.

43. The method of embodiments 1 to 38, or the reporter system of embodiment 39 or 40, wherein the target neoantigen is introduced to the APC by the pulsing of peptide pools.

44. The method of embodiments 1 to 38, or the reporter system of embodiment 39 or 40, wherein the reporter T cell is a primary T cell.

45. The method of embodiments 1 to 38, or the reporter system of embodiment 39 or 40, wherein the reporter cell is from an immortalized T cell line.

46. The method of embodiments 1 to 38, or the reporter system of embodiment 39 or 40, wherein the TCR expression cassette comprises a full-length TCR sequence.

47. The method of embodiments 1 to 38, or the reporter system of embodiment 39 or 40, wherein the reporter T cell expresses any or all protein components of the TCR signaling complex or downstream signaling components.

48. The method of embodiments 1 to 38, or the reporter system of embodiment 39 or 40, wherein the reporter T cell expresses one or more components selected from the group consisting of CD3, CD4, CD8a, and CD8b.

49. The method or the reporter system of embodiment 48, wherein the reporter T cell is modified to enhance the activity of the one or more protein components.

50. The method of embodiments 1 to 38, or the reporter system of embodiment 39 or 40, wherein the TCR expression cassette is cloned into a non-viral gene transfer vector.

51. The method or the reporter system of embodiment 50, wherein the TCR expression cassette is cloned into a transposon.

52. The method of embodiments 1 to 38, or the reporter system of embodiment 39 or 40, wherein the APC is a classical professional APC (such as DC).

53. The method of embodiments 1 to 38, or the reporter system of embodiment 39 or 40, wherein the APC is an artificial APC.

54. The method of embodiments 1 to 38, or the reporter system of embodiment 39 or 40, wherein the APC endogenously express an HLA allele.

55. The method of embodiments 1 to 38, or the reporter system of embodiment 39 or 40, wherein the APC comprises a transgenic HLA expression plasmid.

56. The method of embodiments 1 to 38, or the reporter system of embodiment 39 or 40, wherein the APC expresses multiple transgenic HLA alleles in a single cell.

57. The method of embodiments 1 to 38, or the reporter system of embodiment 39 or 40, wherein the APC expresses a co-stimulatory molecule.

58. The method or the reporter system of embodiment 57, wherein the co-stimulatory molecule is one or more selected from the group consisting of 4-1BBL, CD40, CD80, CD86, or OX40L.

59. The method of embodiment 18, wherein a gene signature for identifying neoantigen reactive TCRs from ex vivo expanded TIL includes one or more gene(s) selected from the group consisting of XCL2, XCL1, IL2, CSF2, IFNG, CCL4, CCL4L2, TNF, CCL3, RGCC, TNFSF9, DUSP2, NFKBID, MIR155HG, NR4A3, EVI2A, CRTAM, ZBED2, FABP5, PIM3, NR4A1, IL10, TNFSF14, NR4A2, LINC00892, ZFP36L1, GZMB, MYC, SPRY1, KDM6B, EGR2, PHLDA1, PPP1R2, VSIR, REL, PRDX1, SLA, CYTOR, DDX21, IER3, PGAM1, NAMPT, HSP90AB1, IL23A, FAM107B, BCL2A1, ZEB2, ZBTB32, BTG2, GADD45B, RILPL2, SEMA7A, TGIF1, SRGN, RAN, CFLAR, MAT2A, SIAH2, PRNP, RNF19A, FASLG, NME1, EVI2B, HSPH1, NOP16, CSRNP1, and TAGAP.

60. A recombinant vector comprising a polycistronic expression cassette, wherein the polycistronic expression cassette comprises a transcriptional regulatory element operably linked to a polycistronic polynucleotide that comprises:
a. a first polynucleotide sequence that encodes a T cell receptor (TCR) alpha chain comprising an alpha chain variable (Vα) region and an alpha chain constant (Cα) region;
b. a second polynucleotide sequence that comprises a first 2A element;
c. a third polynucleotide sequence that encodes a TCR beta chain comprising a beta chain variable (Vβ) region and a beta chain constant (Cβ) region;
d. a fourth polynucleotide sequence that comprises a second 2A element; and
e. a fifth polynucleotide sequence that encodes a fusion protein that comprises IL-15, or a functional fragment or functional variant thereof, and IL-15Rα, or a functional fragment or functional variant thereof.

61. The recombinant vector of embodiment 60, wherein the polycistronic polynucleotide comprises the first, the second, the third, the fourth, and the fifth polynucleotide sequence in any order from 5′ to 3′.

62. The recombinant vector of embodiment 60 or 61, wherein the TCR alpha chain comprises an amino acid sequence at least 90%, 91%, 92%, 93%, 94%, 95%, 96%, 97%, 98%, 99%, or 100% identical to the amino acid sequence of TCR alpha chain sequences disclosed in Tables 1-3, and wherein the TCR beta chain comprises an amino acid sequence at least 90%, 91%, 92%, 93%, 94%, 95%, 96%, 97%, 98%, 99%, or 100% identical to the amino acid sequence of TCR beta chain sequences disclosed in Tables 1-3.

63. A population of cells that comprise the recombinant vector of any one of embodiments 60 to 62.

64. A neoantigen-reactive TCR, or an antigen-binding portion thereof, wherein the neoantigen-reactive TCR or the antigen-binding portion thereof can bind to a neoantigen/HLA complex, wherein the neoantigen comprises one of the EGFR L858R sequences provided in Table 7, and wherein the HLA is HLA-A*11:01.

1. A method for identifying a neoantigen-reactive T cell receptor (TCR), comprising:
i) co-culturing
a) a reporter T cell comprising a TCR expression cassette, and
b) an antigen presenting cell (APC) that expresses a target neoantigen sequence and a matched human leukocyte antigen (HLA) sequence; and
ii) identifying a positive reporter signal in the reporter T cell to identify a neoantigen-reactive TCR.

2. A method for identifying a neoantigen-reactive TCR, comprising:
i. obtaining single-cell gene expression profiles from a population of tumor infiltrating lymphocytes (TIL) isolated from a patient sample,
ii. performing bioinformatics analyses on the single cell gene expression data to identify TCR clonotypes of interests,
iii. creating recombinant TCR sequences;
iv. preparing a reporter T cell comprising a TCR expression cassette encoding a TCR sequence reconstructed from paired TCR α and β chain sequences identified from the clonotype of interest in step ii,
v. preparing a tandem minigene (TMG) expression vector;
vi. analyzing the patient sequencing data to identify class I and class II HLA alleles and preparing HLA expression vectors comprising the class I HLA and class II HLA allele sequences;
vii. preparing an antigen presenting cell (APC) comprising transfecting the TMG expression vector and one or more HLA expression vectors into a cell wherein each transfection condition comprises a TMG and one or two HLA types;
viii. co-culturing the reporter T cell in step iii with the APC of step vii,
ix. identifying a positive reporter activity in the reporter T cell to identify a neoantigen-reactive TCR.

3. The method of claim 2, further comprising obtaining whole exome sequence (WES) data from the patient sample and analyzing the WES data to identify class I and class II HLA alleles.

4. The method of claim 2, wherein the performing bioinformatics analysis further comprises clustering the TCR clonotypes and to select a clonotype of interest.

5. The method of claim 2, wherein the creating recombinant TCR sequences comprises designing alpha and beta TCR sequences in silico.

6. The method of claim 2, wherein the TMG comprises nucleic acid sequences for the expression of concatenated amino acid sequences of non-synonymous single nucleotide variants (SNVs).

7. The method of claim 2, wherein the clustering comprises grouping the TCT clonotypes by CD8 or CD4 expression, gene function of differentially expressed genes, and the level of expression of each TCR.

8. The method of claim 2, further comprising culturing the reporter T cell having the identified neoantigen-reactive TCR with one or more peptides representing the non-synonymous single nucleotide variants (SNVs) present in the TMG.

9. The method of claim 1, wherein the APC is a COS-7 cell.

10. The method of claim 1 or 2, wherein the reporter T cell is an immortalized T cell line.

11. The method of claim 10, wherein the immortalized T cell is a Jurkat cell.

12. The method of claim 11, wherein the Jurkat cell is a Jurkat NFAT cell.

13. The method of any one of claims 1 to 12, wherein the TCR expression cassette comprises a TCR sequence reconstructed from TCR α and β chain sequences identified from tumor infiltrating lymphocytes (TILs) isolated from a tumor sample, and wherein the target neoantigen sequence and the matched HLA sequence are identified from the same tumor sample.

14. The method of any one of claims 1 to 13, wherein the method comprises identifying TCR sequences from TILs isolated from a tumor sample.

15. The method of claim 7, wherein the method further comprises identifying somatic mutations in the tumor sample and determining the germline HLA typing of the tumor sample.

16. A method of identifying a neoantigen-reactive T cell receptor (TCR), comprising:
i) obtaining TCR α and β chain sequences from tumor infiltrating lymphocytes (TILs) isolated from a patient sample;
ii) obtaining neoantigen sequences comprising somatic mutations present in the tumor sample, and the germline HLA typing of the patient sample;
iii) co-culturing
a) a reporter T cell expressing a TCR sequence reconstructed from the TCR α and 3 chain sequences obtained in step i), and
b) an antigen presenting cell (APC) that expresses a neoantigen sequence and a matched human leukocyte antigen (HLA) sequence obtained in step ii); and
iv) evaluating the reporter activity in the reporter T cell to identify a neoantigen-reactive TCR.

17. The method of claim 16, wherein the APC is a COS-7 cell, and the reporter T cell is a Jurkat NFAT cell.

18. The method of any one of claims 13 to 17, wherein the isolated TILs are first expanded ex vivo and then co-cultured with APCs modified to express relevant HLA alleles and antigens obtained from the tumor sample.

19. The method of any one of claims 1 to 18, wherein the reporter T cell comprises a reporter system that is activated by the binding of the TCR to the neoantigen.

20. The method of any one of claims 1 to 19, wherein the reporter T cell and the APC are co-cultured in a ratio of 1:16, 1:8, 1:4, 1:2, 1:1, 2:1, 4:1, 8:1, or 16:1.

21. The method of any one of claims 1 to 20, wherein the reporter T cell and the APC are co-cultured for 1-48 hours.

22. A TCR, or an antigen-binding portion thereof, isolated according to the method of any one of claims 1 to 21.

23. A TCR, or an antigen-binding portion thereof, comprising a sequence selected from the group consisting of SEQ ID NOs: 1-36 (the sequences provided in Tables 1-3).

24. A neoantigen/HLA complex, wherein the neoantigen comprises a sequence selected from the group consisting of SEQ ID NOs: 67 to 292 and wherein the HLA comprises a sequence selected from a group consisting of SEQ ID NOs: 58 to 66.

25. The neoantigen/HLA complex of claim 24, wherein the neoantigen is SEQ ID NO: 238 and the HLA sequence is SEQ ID NOs: 60 and 62 or SEQ ID NOs: 61 and 62.

26. The neoantigen/HLA complex of claim 24, wherein the neoantigen is SEQ ID NO: 142 and the HLA sequence is SEQ ID NO: 58.

27. The neoantigen/HLA complex of claim 24, wherein the neoantigen is SEQ ID NO: 151 and the HLA sequence is SEQ ID NOs: 65 and 63.

28. The neoantigen/HLA complex of claim 24, wherein the neoantigen is SEQ ID NO: 162 and the HLA sequence is SEQ ID NO: 59.

29. A recombinant vector expressing the T cell receptor, or an antigen-binding portion thereof, of claim 22 or 23.

30. A polynucleotide encoding an amino acid sequence that is at least 90%, 95%, 96%, 97%, 98%, 99%, or 100% identical to an amino acid sequence selected from the group consisting of SEQ ID NOs: 1-36 (the sequences provided in Tables 1-3).

31. A population of cells that comprise the recombinant vector of claim 29, or the polynucleotide of claim 30.

32. The population of cells of claim 31, wherein the recombinant vector or the polynucleotide is integrated into the genome of the population of cells.

33. The population of cells of claim 31 or 32, wherein the cells are immune effector cells.

34. The population of cells of claim 33, wherein the immune effector cells are selected from the group consisting of T cells, natural killer (NK) cells, B cells, mast cells, and myeloid-derived phagocytes.

35. A pharmaceutical composition comprising the population of cells of any one of claims 31 to 34, and a pharmaceutically acceptable carrier.

36. A method of preparing a medicament for the treatment or prevention of a medical condition, the method comprising preparing the population of cells any one of claims 31 to 34.

37. A method of treating a disease or medical condition, the method comprising administering the pharmaceutical composition of claim 23 to a patient in need.

38. The method of claim 36 or 37, wherein the disease or medical condition is a cancer.

39. A co-culture reporter system for identifying a neoantigen-reactive T cell receptor (TCR), comprising:
i) a reporter T cell comprising a TCR expression cassette, co-cultured with
ii) an antigen presenting cell (APC) that expresses a target neoantigen sequence and a matched human leukocyte antigen (HLA) sequence.

40. The co-culture reporter system of claim 39, wherein the APC is a COS-7 cell, and the reporter T cell is a Jurkat NFAT cell.

41. The method of claims 1 to 38, or the reporter system of claim 39 or 40, wherein the target neoantigen is expressed in an antigen encoding plasmid.

42. The method or the reporter system of claim 41, wherein the antigen encoding plasmid is a Tandem Minigene (TMG) plasmid.

43. The method of claims 1 to 38, or the reporter system of claim 39 or 40, wherein the target neoantigen is introduced to the APC by the pulsing of peptide pools.

44. The method of claims 1 to 38, or the reporter system of claim 39 or 40, wherein the reporter T cell is a primary T cell.

45. The method of claims 1 to 38, or the reporter system of claim 39 or 40, wherein the reporter cell is from an immortalized T cell line.

46. The method of claims 1 to 38, or the reporter system of claim 39 or 40, wherein the TCR expression cassette comprises a full-length TCR sequence.

47. The method of claims 1 to 38, or the reporter system of claim 39 or 40, wherein the reporter T cell expresses any or all protein components of the TCR signaling complex or downstream signaling components.

48. The method of claims 1 to 38, or the reporter system of claim 39 or 40, wherein the reporter T cell expresses one or more components selected from the group consisting of CD3, CD4, CD8a, and CD8b.

49. The method or the reporter system of claim 48, wherein the reporter T cell is modified to enhance the activity of the one or more protein components.

50. The method of claims 1 to 38, or the reporter system of claim 39 or 40, wherein the TCR expression cassette is cloned into a non-viral gene transfer vector.

51. The method or the reporter system of claim 50, wherein the TCR expression cassette is cloned into a transposon.

52. The method of claims 1 to 38, or the reporter system of claim 39 or 40, wherein the APC is a classical professional APC (such as DC).

53. The method of claims 1 to 38, or the reporter system of claim 39 or 40, wherein the APC is an artificial APC.

54. The method of claims 1 to 38, or the reporter system of claim 39 or 40, wherein the APC endogenously express an HLA allele.

55. The method of claims 1 to 38, or the reporter system of claim 39 or 40, wherein the APC comprises a transgenic HLA expression plasmid.

56. The method of claims 1 to 38, or the reporter system of claim 39 or 40, wherein the APC expresses multiple transgenic HLA alleles in a single cell.

57. The method of claims 1 to 38, or the reporter system of claim 39 or 40, wherein the APC expresses a co-stimulatory molecule.

58. The method or the reporter system of claim 57, wherein the co-stimulatory molecule is one or more selected from the group consisting of 4-1BBL, CD40, CD80, CD86, or OX40L.

59. The method of claim 18, wherein a gene signature for identifying neoantigen reactive TCRs from ex vivo expanded TIL includes one or more gene(s) selected from the group consisting of XCL2, XCL1, IL2, CSF2, IFNG, CCL4, CCL4L2, TNF, CCL3, RGCC, TNFSF9, DUSP2, NFKBID, MIR155HG, NR4A3, EVI2A, CRTAM, ZBED2, FABP5, PIM3, NR4A1, IL10, TNFSF14, NR4A2, LINC00892, ZFP36L1, GZMB, MYC, SPRY1, KDM6B, EGR2, PHLDA1, PPP1R2, VSIR, REL, PRDX1, SLA, CYTOR, DDX21, IER3, PGAM1, NAMPT, HSP90AB1, IL23A, FAM107B, BCL2A1, ZEB2, ZBTB32, BTG2, GADD45B, RILPL2, SEMA7A, TGIF1, SRGN, RAN, CFLAR, MAT2A, SIAH2, PRNP, RNF19A, FASLG, NME1, EVI2B, HSPH1, NOP16, CSRNP1, and TAGAP.

60. A recombinant vector comprising a polycistronic expression cassette, wherein the polycistronic expression cassette comprises a transcriptional regulatory element operably linked to a polycistronic polynucleotide that comprises:
a. a first polynucleotide sequence that encodes a T cell receptor (TCR) alpha chain comprising an alpha chain variable (Vα) region and an alpha chain constant (Cα) region;
b. a second polynucleotide sequence that comprises a first 2A element;
c. a third polynucleotide sequence that encodes a TCR beta chain comprising a beta chain variable (Vβ) region and a beta chain constant (Cβ) region;
d. a fourth polynucleotide sequence that comprises a second 2A element; and
e. a fifth polynucleotide sequence that encodes a fusion protein that comprises IL-15, or a functional fragment or functional variant thereof, and IL-15Rα, or a functional fragment or functional variant thereof.

61. The recombinant vector of claim 60, wherein the polycistronic polynucleotide comprises the first, the second, the third, the fourth, and the fifth polynucleotide sequence in any order from 5′ to 3′.

62. The recombinant vector of claim 60 or 61, wherein the TCR alpha chain comprises an amino acid sequence at least 90%, 91%, 92%, 93%, 94%, 95%, 96%, 97%, 98%, 99%, or 100% identical to the amino acid sequence of TCR alpha chain sequences disclosed in Tables 1-3, and wherein the TCR beta chain comprises an amino acid sequence at least 90%, 91%, 92%, 93%, 94%, 95%, 96%, 97%, 98%, 99%, or 100% identical to the amino acid sequence of TCR beta chain sequences disclosed in Tables 1-3.

63. A population of cells that comprise the recombinant vector of any one of claims 60 to 62.

64. A neoantigen-reactive TCR, or an antigen-binding portion thereof, wherein the neoantigen-reactive TCR or the antigen-binding portion thereof can bind to a neoantigen/HLA complex, wherein the neoantigen comprises one of the EGFR L858R sequences provided in Table 7, and wherein the HLA is HLA-A*11:01.


SEQUENCE LISTING (300 sequences)

SEQ ID NO: 1 - protein, 6 aa, synthetic construct
  REGION 1..6: note = Description of sequence: CDR1-alpha
DRGSQS

SEQ ID NO: 2 - protein, 6 aa, synthetic construct
  REGION 1..6: note = Description of sequence: CDR2-alpha
IYSNGD

SEQ ID NO: 3 - protein, 9 aa, synthetic construct
  REGION 1..9: note = Description of sequence: CDR3-alpha
AVYNNNDMR

SEQ ID NO: 4 - protein, 119 aa, synthetic construct
  REGION 1..119: note = Description of sequence: V-alpha without signal peptide (SignalP)
QQKEVEQNSGPLSVPEGAIASLNCTYSDRGSQSFFWYRQYSGKSPELIMSIYSNGDKEDGRFTAQLNKASQYVSLLIRDSQPSDSATYLCAVYNNNDMRFGAGTRLTVKPNIQNPEPAV

SEQ ID NO: 5 - protein, 140 aa, synthetic construct
  REGION 1..140: note = Description of sequence: V-alpha only (without the Constant)
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVYNNNDMRFGAGTRLTVKPNIQNPEPAV

SEQ ID NO: 6 - protein, 277 aa, synthetic construct
  REGION 1..277: note = Description of sequence: -alpha chain with WT signal peptide and constant C-alpha
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVYNNNDMRFGAGTRLTVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 7 - protein, 5 aa, synthetic construct
  REGION 1..5: note = Description of sequence: CDR1-beta
SGHTA

SEQ ID NO: 8 - protein, 6 aa, synthetic construct
  REGION 1..6: note = Description of sequence: CDR2-beta
FQGTGA

SEQ ID NO: 9 - protein, 15 aa, synthetic construct
  REGION 1..15: note = Description of sequence: CDR3-beta
ASSFLAGHLYTGELF

SEQ ID NO: 10 - protein, 120 aa, synthetic construct
  REGION 1..120: note = Description of sequence: V-beta without signal peptide (SignalP)
GVSQTPSNKVTEKGKYVELRCDPISGHTALYWYRQSLGQGPEFLIYFQGTGAADDSGLPNDRFFAVRPEGSVSTLKIQRTERGDSAVYLCASSFLAGHLYTGELFFGEGSRLTVLEDLRN

SEQ ID NO: 11 - protein, 141 aa, synthetic construct
  REGION 1..141: note = Description of sequence: V-beta (without the Constant)
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSFLAGHLYTGELFFGEGSRLTVLEDLRN

SEQ ID NO: 12 - protein, 314 aa, synthetic construct
  REGION 1..314: note = Description of sequence: -beta chain with WT signal peptide and constant C-beta
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSFLAGHLYTGELFFGEGSRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 13 - protein, 6 aa, synthetic construct
  REGION 1..6: note = Description of sequence: CDR1-alpha
NSASDY

SEQ ID NO: 14 - protein, 7 aa, synthetic construct
  REGION 1..7: note = Description of sequence: CDR2-alpha
IRSNMDK

SEQ ID NO: 15 - protein, 8 aa, synthetic construct
  REGION 1..8: note = Description of sequence: CDR3-alpha
AETPDKLI

SEQ ID NO: 16 - protein, 115 aa, synthetic construct
  REGION 1..115: note = Description of sequence: V-alpha without signal peptide (SignalP)
VGLHLPTLSVQEGDNSIINCAYSNSASDYFIWYKQESGKGPQFIIDIRSNMDKRQGQRVTVLLNKTVKHLSLQIAATQPGDSAVYFCAETPDKLIFGTGTRLQVFPNIQNPEPAV

SEQ ID NO: 17 - protein, 138 aa, synthetic construct
  REGION 1..138: note = Description of sequence: V-alpha only (without the Constant)
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAETPDKLIFGTGTRLQVFPNIQNPEPAV

SEQ ID NO: 18 - protein, 275 aa, synthetic construct
  REGION 1..275: note = Description of sequence: -alpha chain with WT signal peptide and constant C-alpha
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAETPDKLIFGTGTRLQVFPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 19 - protein, 5 aa, synthetic construct
  REGION 1..5: note = Description of sequence: CDR1-beta
SEHNR

SEQ ID NO: 20 - protein, 6 aa, synthetic construct
  REGION 1..6: note = Description of sequence: CDR2-beta
FQNEAQ

SEQ ID NO: 21 - protein, 11 aa, synthetic construct
  REGION 1..11: note = Description of sequence: CDR3-beta
ASSGGGDSPLH

SEQ ID NO: 22 - protein, 118 aa, synthetic construct
  REGION 1..118: note = Description of sequence: V-beta without signal peptide (SignalP)
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSGGGDSPLHFGNGTRLTVTEDLRN

SEQ ID NO: 23 - protein, 137 aa, synthetic construct
  REGION 1..137: note = Description of sequence: V-beta (without the Constant)
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSGGGDSPLHFGNGTRLTVTEDLRN

SEQ ID NO: 24 - protein, 310 aa, synthetic construct
  REGION 1..310: note = Description of sequence: -beta chain with WT signal peptide and constant C-beta
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSGGGDSPLHFGNGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - protein, 6 aa, synthetic construct
  REGION 1..6: note = Description of sequence: CDR1-alpha
YGATPY

SEQ ID NO: 26 - protein, 8 aa, synthetic construct
  REGION 1..8: note = Description of sequence: CDR2-alpha
YFSGDTLV

SEQ ID NO: 27 - protein, 11 aa, synthetic construct
  REGION 1..11: note = Description of sequence: CDR3-alpha
AVGGGGADGLT

SEQ ID NO: 28 - protein, 121 aa, synthetic construct
  REGION 1..121: note = Description of sequence: V-alpha without signal peptide (SignalP)
QSVTQPDIHITVSEGASLELRCNYSYGATPYLFWYVQSPGQGLQLLLKYFSGDTLVQGIKGFEAEFKRSQSSFNLRKPSVHWSDAAEYFCAVGGGGADGLTFGKGTHLIIQPNIQNPEPAV

SEQ ID NO: 29 - protein, 141 aa, synthetic construct
  REGION 1..141: note = Description of sequence: V-alpha only (without the Constant)
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGGGGADGLTFGKGTHLIIQPNIQNPEPAV

SEQ ID NO: 30 - protein, 278 aa, synthetic construct
  REGION 1..278: note = Description of sequence: -alpha chain with WT signal peptide and constant C-alpha
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGGGGADGLTFGKGTHLIIQPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 31 - protein, 5 aa, synthetic construct
  REGION 1..5: note = Description of sequence: CDR1-beta
MDHEN

SEQ ID NO: 32 - protein, 6 aa, synthetic construct
  REGION 1..6: note = Description of sequence: CDR2-beta
SYDVKM

SEQ ID NO: 33 - protein, 13 aa, synthetic construct
  REGION 1..13: note = Description of sequence: CDR3-beta
ATLWYRQGADGYT

SEQ ID NO: 34 - protein, 112 aa, synthetic construct
  REGION 1..112: note = Description of sequence: V-beta without signal peptide (SignalP)
SRYLVKRTGEKVFLECVQDMDHENMFWYRQDPGLGLRLIYFSYDVKMKEKGDIPEGYSVSREKKERFSLILESASTNQTSMYLCATLWYRQGADGYTFGSGTRLTVVEDLRN

SEQ ID NO: 35 - protein, 138 aa, synthetic construct
  REGION 1..138: note = Description of sequence: V-beta (without the Constant)
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCATLWYRQGADGYTFGSGTRLTVVEDLRN

SEQ ID NO: 36 - protein, 311 aa, synthetic construct
  REGION 1..311: note = Description of sequence: -beta chain with WT signal peptide and constant C-beta
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCATLWYRQGADGYTFGSGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 37 - protein, 137 aa, Mus musculus
  REGION 1..137: note = Description of sequence: C-alpha (murine, degenerate)
  VARIANT 1: note = X at position 1 is Asn, Asp, His, or Tyr;
  VARIANT 48: note = X at position 48 is Thr or Cys;
  VARIANT 112: note = X at position 112 is Ser, Ala, Val, Leu, Ile, Pro, Phe, Met, or Trp;
  VARIANT 114: note = X at position 114 is Met, Ala, Val, Leu, Ile, Pro, Phe, or Trp;
  VARIANT 115: note = X at position 115 is Gly, Ala, Val, Leu, Ile, Pro, Phe, Met, or Trp
XIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 38 - DNA, 411 bp, Mus musculus
  misc_feature 1..411: note = Description of sequence: C-alpha (murine, degenerate) (exemplary nucleotide sequence)
  source 1..411: mol_type = genomic DNA
  misc_feature 1..3: note = Together NNN at positions 1-3 make up a codon that encodes Asn, Asp, His, or Tyr;
  misc_feature 142..144: note = Together NNN at positions 142-144 make up a codon that encodes Thr or Cys;
  misc_feature 334..336: note = Together NNN at positions 334-336 make up a codon that encodes Ser, Ala, Val, Leu, Ile, Pro, Phe, Met, or Trp;
  misc_feature 340..342: note = Together NNN at positions 340-342 make up a codon that encodes Met, Ala, Val, Leu, Ile, Pro, Phe, or Trp;
  misc_feature 343..345: note = Together NNN at positions 343-345 make up a codon that encodes Gly, Ala, Val, Leu, Ile, Pro, Phe, Met, or Trp
  variation 1..3: note = n is a, c, g, or t
  variation 142..144: note = n is a, c, g, or t
  variation 334..336: note = n is a, c, g, or t
  variation 340..345: note = n is a, c, g, or t
nnnatccagaatcccgagcctgcggtgtaccagctgaaggacccccgctctcaggatagcacactgtgcctgttcaccgactttgatagccagatcaacgtgcctaaaacaatggagtccggcaccttcatcaccgacaagnnngtgctggatatgaaagcgatggactccaagtctaacggcgcgatcgcgtggtccaatcagacatctttcacctgccaggatatcttcaaggagacaaacgcgacctatccttcctctgacgtgccatgtgatgcgacactgaccgagaagagcttcgagacagacatgaacctgaattttcagaatctgnnngtcnnnnnnctgagaatcctgctgctgaaggtggcgggctttaatctgctgatgacactgcggctgtggagttcc

SEQ ID NO: 39 - protein, 137 aa, Mus musculus
  REGION 1..137: note = Description of sequence: C-alpha (murine, cysteine- and LIV-substituted)
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 40 - DNA, 411 bp, Mus musculus
  misc_feature 1..411: note = Description of sequence: C-alpha (murine, cysteine- and LIV-substituted) (exemplary nucleotide sequence)
  source 1..411: mol_type = genomic DNA
aacatccagaatcccgagcctgcggtgtaccagctgaaggacccccgctctcaggatagcacactgtgcctgttcaccgactttgatagccagatcaacgtgcctaaaacaatggagtccggcaccttcatcaccgacaagtgcgtgctggatatgaaagcgatggactccaagtctaacggcgcgatcgcgtggtccaatcagacatctttcacctgccaggatatcttcaaggagacaaacgcgacctatccttcctctgacgtgccatgtgatgcgacactgaccgagaagagcttcgagacagacatgaacctgaattttcagaatctgctggtcatcgtgctgagaatcctgctgctgaaggtggcgggctttaatctgctgatgacactgcggctgtggagttcc

SEQ ID NO: 41 - protein, 137 aa, Mus musculus
  REGION 1..137: note = Description of sequence: C-alpha (murine, LIV substituted)
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 42 - DNA, 411 bp, Mus musculus
  misc_feature 1..411: note = Description of sequence: C-alpha (murine, LIV substituted) (exemplary nucleotide sequence)
  source 1..411: mol_type = genomic DNA
aacatccagaatcccgagcctgcggtgtaccagctgaaggacccccgctctcaggatagcacactgtgcctgttcaccgactttgatagccagatcaacgtgcctaaaacaatggagtccggcaccttcatcaccgacaagaccgtgctggatatgaaagcgatggactccaagtctaacggcgcgatcgcgtggtccaatcagacatctttcacctgccaggatatcttcaaggagacaaacgcgacctatccttcctctgacgtgccatgtgatgcgacactgaccgagaagagcttcgagacagacatgaacctgaattttcagaatctgctggtcatcgtgctgagaatcctgctgctgaaggtggcgggctttaatctgctgatgacactgcggctgtggagttcc

SEQ ID NO: 43 - protein, 137 aa, Mus musculus
  REGION 1..137: note = Description of sequence: C-alpha (murine, cysteine-substituted)
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 44 - protein, 137 aa, Mus musculus
  REGION 1..137: note = Description of sequence: C-alpha (murine, wild type)
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 45 - protein, 141 aa, Homo sapiens
  REGION 1..141: note = Description of sequence: C-alpha (human, degenerate)
  VARIANT 1: note = X at position 1 is Asn, Asp, His, or Tyr
  VARIANT 48: note = X at position 48 is Thr or Cys;
  VARIANT 116: note = X at position 116 is Ser, Ala, Val, Leu, Ile, Pro, Phe, Met, or Trp;
  VARIANT 118: note = X at position 118 is Met, Ala, Val, Leu, Ile, Pro, Phe, or Trp;
  VARIANT 119: note = X at position 119 is Gly, Ala, Val, Leu, Ile, Pro, Phe, Met, or Trp
XIQNPDPAVYQLRDSKSSDKSVCLFTDFDSQTNVSQSKDSDVYITDKXVLDMRSMDFKSNSAVAWSNKSDFACANAFNNSIIPEDTFFPSPESSCDVKLVEKSFETDTNLNFQNLXVXXFRILLLKVAGFNLLMTLRLWSS

SEQ ID NO: 46 - protein, 141 aa, Homo sapiens
  REGION 1..141: note = Description of sequence: C-alpha (human, cysteine- and LIV-substituted; degenerate at position 1)
  VARIANT 1: note = X at position 1 is Asn, Asp, His, or Tyr
XIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVFRILLLKVAGFNLLMTLRLWSS

SEQ ID NO: 47 - protein, 141 aa, Homo sapiens
  REGION 1..141: note = Description of sequence: C-alpha (human, LIV-substituted; degenerate at position 1)
  VARIANT 1: note = X at position 1 is Asn, Asp, His, or Tyr
XIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLLVIVFRILLLKVAGFNLLMTLRLWSS

SEQ ID NO: 48 - protein, 141 aa, Homo sapiens
  REGION 1..141: note = Description of sequence: C-alpha (human, cysteine-substituted; degenerate at position 1)
  VARIANT 1: note = X at position 1 is Asn, Asp, His, or Tyr
XIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 49 - protein, 141 aa, Homo sapiens
  REGION 1..141: note = Description of sequence: C-alpha (human, wild type; degenerate at position 1)
  VARIANT 1: note = X at position 1 is Asn, Asp, His, or Tyr
XIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 173 aa, Mus musculus
  REGION 1..173: note = Description of sequence: C-beta (murine, degenerate)
  VARIANT 57: note = X at position 57 is Ser or Cys
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 51 - DNA, 519 bp, Mus musculus
  misc_feature 1..519: note = Description of sequence: C-beta (murine, degenerate) (exemplary nucleotide sequence)
  source 1..519: mol_type = genomic DNA
  misc_feature 169..171: note = Together positions 169-171 make up a codon that encodes Ser or Cys, i.e. TCN or TGY
  variation 171: note = If position 170 is C, N can be any amino acid, if position 170 is G, N is either T or C
gaggacctgaggaacgtgaccccacctaaagtgagcctgttcgagccatccaaggcggagatcgcgaataagcagaaagcgaccctggtgtgcctggcgaggggcttctttcccgatcacgtggagctgtcctggtgggtgaacggcaaagaggtgcactctggcgtgtsnacagaccctcaggcgtacaaggagagcaattactcctattgtctgtctagcagactgagggtgagcgcgaccttttggcacaacccccggaatcacttccgctgccaggtgcagtttcacggcctgtccgaggaggataaatggcctgagggctctccaaagcccgtgacacagaatatcagcgcggaggcgtggggaagagcggactgtggcattacaagcgcgtcctatcagcagggcgtgctgtccgcgaccatcctgtacgagattctgctgggcaaggcgacactgtatgcggtgctggtgtccaccctggtggtcatggcgatggtgaagaggaaaaactct

SEQ ID NO: 52 - protein, 173 aa, Mus musculus
  REGION 1..173: note = Description of sequence: C-beta (murine, cysteine-substituted)
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 53 - DNA, 519 bp, Mus musculus
  misc_feature 1..519: note = Description of sequence: C-beta (murine, cysteine-substituted) (exemplary nucleotide sequence)
  source 1..519: mol_type = genomic DNA
gaggacctgaggaacgtgaccccacctaaagtgagcctgttcgagccatccaaggcggagatcgcgaataagcagaaagcgaccctggtgtgcctggcgaggggcttctttcccgatcacgtggagctgtcctggtgggtgaacggcaaagaggtgcactctggcgtgtgcacagaccctcaggcgtacaaggagagcaattactcctattgtctgtctagcagactgagggtgagcgcgaccttttggcacaacccccggaatcacttccgctgccaggtgcagtttcacggcctgtccgaggaggataaatggcctgagggctctccaaagcccgtgacacagaatatcagcgcggaggcgtggggaagagcggactgtggcattacaagcgcgtcctatcagcagggcgtgctgtccgcgaccatcctgtacgagattctgctgggcaaggcgacactgtatgcggtgctggtgtccaccctggtggtcatggcgatggtgaagaggaaaaactct

SEQ ID NO: 54 - protein, 173 aa, Mus musculus
  REGION 1..173: note = Description of sequence: C-beta (murine, wild type)
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 55 - protein, 179 aa, Homo sapiens
  REGION 1..179: note = Description of sequence: C-beta (human, degenerate)
  VARIANT 57: note = X at position 57 is Ser or Cys
EDLKNVFPPEVAVFEPSEAEISHTQKATLVCLATGFYPDHVELSWWVNGKEVHSGVXTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 56 - protein, 179 aa, Homo sapiens
  REGION 1..179: note = Description of sequence: C-beta (human, cysteine-substituted)
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 57 - protein, 179 aa, Homo sapiens
  REGION 1..179: note = Description of sequence: C-beta (human, wild type)
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 58 - protein, 365 aa, synthetic construct
  REGION 1..365: note = Description of sequence: HLA-A*03:01; Type of sequence: Restricting HLA
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 59 - protein, 362 aa, synthetic construct [sequence omitted]

SEQ ID NO: 60 - protein, 260 aa, synthetic construct
  REGION 1..260: note = Description of sequence: HLA-DPA1*01:03 ; Type of sequence: Restricting HLA
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 61 - protein, 260 aa, synthetic construct [sequence omitted]

SEQ ID NO: 62 - protein, 258 aa, synthetic construct [sequence omitted]

SEQ ID NO: 63 - protein, 254 aa, synthetic construct
  REGION 1..254: note = Description of sequence: HLA-DRA*01:01; Type of sequence: Restricting HLA
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 64 - protein, 266 aa, synthetic construct
  REGION 1..266: note = Description of sequence: HLA-DRB1*01:01; Type of sequence: Restricting HLA
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 65 - protein, 266 aa, synthetic construct [sequence omitted]

SEQ ID NO: 66 - protein, 266 aa, synthetic construct
  REGION 1..266: note = Description of sequence: HLA-DRB4*01:03; Type of sequence: Restricting HLA
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 67 - protein, 25 aa, synthetic construct
  REGION 1..25: note = Description of sequence: 2599_TWNK_p.D49Y; Type of sequence: Mutation/Peptide
RRRYRKETLQALYMPVLPVTATEIR

SEQ ID NO: 68 - protein, 25 aa, synthetic construct
  REGION 1..25: note = Description of sequence: 2599_SOX6_p.V37E; Type of sequence: Mutation/Peptide
TSREKEEGSDQHEASHLPLHPIMHN

SEQ ID NO: 69 - protein, 25 aa, synthetic construct
  REGION 1..25: note = Description of sequence: 2599_NAA40_p.V146L; Type of sequence: Mutation/Peptide
VLYCYEVQLESKLRRKGLGKFLIQI

SEQ ID NO: 70 - protein, 25 aa, synthetic construct
  REGION 1..25: note = Description of sequence: 2599_ATG2A_p.V1580I; Type of sequence: Mutation/Peptide
TTNLGGPECCLRISLMPLRLNVDQD

SEQ ID NO: 71 - protein, 25 aa, synthetic construct
  REGION 1..25: note = Description of sequence: 2599_SLCO1B3_p.L180R; Type of sequence: Mutation/Peptide
SHMWIYVFMGNMRRGIGETPIVPLG

SEQ ID NO: 72 - protein, 25 aa, synthetic construct
  REGION 1..25: note = Description of sequence: 2599_ERGIC2_p.L176P; Type of sequence: Mutation/Peptide
SQSPNACRIHGHPYVNKVAGNFHIT

SEQ ID NO: 73 - protein, 25 aa, synthetic construct
  REGION 1..25: note = Description of sequence: 2599_CSAD_p.P88L; Type of sequence: Mutation/Peptide
PSARRSYLCTLPLALLSREILMADS

SEQ ID NO: 74 - protein, 25 aa, synthetic construct
  REGION 1..25: note = Description of sequence: 2599_CSAD_p.P19L; Type of sequence: Mutation/Peptide
SSFLLSYLCTLPLALLSREILMADS

SEQ ID NO: 75 - protein, 25 aa, synthetic construct
  REGION 1..25: note = Description of sequence: 2599_NAV3_p.R1575W; Type of sequence: Mutation/Peptide
EEKAHSEQIHKLWRELVASQEKVAT

SEQ ID NO: 76 - protein, 25 aa, synthetic construct
  REGION 1..25: note = Description of sequence: 2599_ABHD13_p.R27C; Type of sequence: Mutation/Peptide
LIALASWSWALCCISLLPLIVTFHL

SEQ ID NO: 77 - protein, 25 aa, synthetic construct
  REGION 1..25: note = Description of sequence: 2599_NBEA_p.P2474T; Type of sequence: Mutation/Peptide
VNDVDLPPWAKKTEDFVRINRMALE

SEQ ID NO: 78 - protein, 25 aa, synthetic construct
  REGION 1..25: note = Description of sequence: 2599_THSD1_p.H644R; Type of sequence: Mutation/Peptide
HVGSRGGPSERSRARNAHFRRTASF

SEQ ID NO: 79 - protein, 25 aa, synthetic construct
  REGION 1..25: note = Description of sequence: 2599_GPC6_p.R95C; Type of sequence: Mutation/Peptide
FENLVEETSHFVCTTFVSRHKKFDE

SEQ ID NO: 80 - protein, 25 aa, synthetic construct
  REGION 1..25: note = Description of sequence: 2599_CERS3_p.G197V; Type of sequence: Mutation/Peptide
EMSFYWSLLFRLVFDVKRKDFLAHI

SEQ ID NO: 81 - protein, 25 aa, synthetic construct
  REGION 1..25: note = Description of sequence: 2599_GLDN_p.V226M; Type of sequence: Mutation/Peptide
GEKGDKGDVSNDMLLAGAKGDQGPP

SEQ ID NO: 82 - protein, 25 aa, synthetic construct
  REGION 1..25: note = Description of sequence: 2599_FAM219B_p.A178V; Type of sequence: Mutation/Peptide
DEDLDLIPPKPMVSSTCSCCWCCLG

SEQ ID NO: 83 - protein, 25 aa, synthetic construct
  REGION 1..25: note = Description of sequence: 2599_BAIAP3_p.H425L; Type of sequence: Mutation/Peptide
ELSTPAATILCLLGAQSNLSPLQLA

SEQ ID NO: 84 - protein, 25 aa, synthetic construct
  REGION 1..25: note = Description of sequence: 2599_CLEC19A_p.F54L; Type of sequence: Mutation/Peptide
LFWMEFKGHCYRLFPLNKTWAEADL

SEQ ID NO: 85 - protein, 25 aa, synthetic construct
  REGION 1..25: note = Description of sequence: 2599_ZPBP2_p.R165G; Type of sequence: Mutation/Peptide
RSCIGRYNDVFFGVLKKILDSLISD

SEQ ID NO: 86 - protein, 25 aa, synthetic construct
  REGION 1..25: note = Description of sequence: 2599_ASB16_p.D125G; Type of sequence: Mutation/Peptide
APLAIATARGYTGCARHLIRQGAEL

SEQ ID NO: 87 - protein, 25 aa, synthetic construct
  REGION 1..25: note = Description of sequence: 2599_ARHGAP27_p.R635H; Type of sequence: Mutation/Peptide
SESSRVDFGSSEHLGSWQEKEEDAR

SEQ ID NO: 88 - protein, 25 aa, synthetic construct
  REGION 1..25: note = Description of sequence: 2599_TACO1_p.R79P; Type of sequence: Mutation/Peptide
RHIKGPKDVERSPIFSKLCLNIRLA

SEQ ID NO: 89 - protein, 25 aa, synthetic construct
  REGION 1..25: note = Description of sequence: 2599_TP53_p.R273C; Type of sequence: Mutation/Peptide
SGNLLGRNSFEVCVCACPGRDRRTE

SEQ ID NO: 90 - protein, 25 aa, synthetic construct
  REGION 1..25: note = Description of sequence: 2599_PLEKHG2_p.S1318L; Type of sequence: Mutation/Peptide
GSWSSAPTSRASLPPPQPQPPPPPA

SEQ ID NO: 91 - protein, 25 aa, synthetic construct
  REGION 1..25: note = Description of sequence: 2599_ZNF229_p.C491W; Type of sequence: Mutation/Peptide
HQKTHTGERPYQWDKCGKGFSHNSY

SEQ ID NO: 92 - protein, 25 aa, synthetic construct
  REGION 1..25: note = Description of sequence: 2599_TRIM33_p.A1026P; Type of sequence: Mutation/Peptide
HSQHYQIPDDFVPDVRLIFKNCERF

SEQ ID NO: 93 - protein, 25 aa, synthetic construct
  REGION 1..25: note = Description of sequence: 2599_MLLT11_p.S25L; Type of sequence: Mutation/Peptide
LFWRMPIPELDLLELEGLGLSDTAT

SEQ ID NO: 94 - protein, 25 aa, synthetic construct
  REGION 1..25: note = Description of sequence: 2599_SMCP_p.C35Y; Type of sequence: Mutation/Peptide
QNQCCQSKGNQCYPPKQNQCCQPKG

SEQ ID NO: 95 - protein, 25 aa, synthetic construct
  REGION 1..25: note = Description of sequence: 2599_LRRN2_p.R329H; Type of sequence: Mutation/Peptide
ITNNPRLSFIHPHAFHHLPQMETLM

SEQ ID NO: 96 - protein, 25 aa, synthetic construct
  REGION 1..25: note = Description of sequence: 2599_HHIPL2_p.R167H; Type of sequence: Mutation/Peptide
RGLQESHGRDGTHFCHLLDLPDKDY

SEQ ID NO: 97 - protein, 25 aa, synthetic construct
  REGION 1..25: note = Description of sequence: 2599_RYR2_p.E4156K; Type of sequence: Mutation/Peptide
VLNYFQPFLGRIKIMGSAKRIERVY

SEQ ID NO: 98 - protein, 25 aa, synthetic construct
  REGION 1..25: note = Description of sequence: 2599_OR2M4_p.V204A; Type of sequence: Mutation/Peptide
TSAFERLLVICCAVMLIFPVSVIIL

SEQ ID NO: 99 - protein, 25 aa, synthetic construct
  REGION 1..25: note = Description of sequence: 2599_OR2T3_p.G238S; Type of sequence: Mutation/Peptide
ILHLIHRMNSAASHRKALATCSSHM

SEQ ID NO: 100 - protein, 25 aa, synthetic construct
  REGION 1..25: note = Description of sequence: 2599_LEPROT_p.G124R; Type of sequence: Mutation/Peptide
LAGNAVIFLTIQRFFLIFGRGDDFS

SEQ ID NO: 101 - protein, 25 aa, synthetic construct
  REGION 1..25: note = Description of sequence: 2599_FPGT_p.C467G; Type of sequence: Mutation/Peptide
LTKAALPAHSFVGSLSLKMNRCLKY

SEQ ID NO: 102 - protein, 25 aa, synthetic construct
  REGION 1..25: note = Description of sequence: 2599_NAPB_p.K246Q; Type of sequence: Mutation/Peptide
EMFPAFTDSRECQLLKKLLEAHEEQ

SEQ ID NO: 103 - protein, 25 aa, synthetic construct
  REGION 1..25: note = Description of sequence: 2599_ITCH_p.N415K; Type of sequence: Mutation/Peptide
QRSQLQGAMQQFKQRFIYGNQDLFA

SEQ ID NO: 104 - protein, 25 aa, synthetic construct
  REGION 1..25: note = Description of sequence: 2599_LONRF2_p.T155M; Type of sequence: Mutation/Peptide
CPRCRRLLHKPVMLPCGLTVCKRCV

SEQ ID NO: 105 - protein, 25 aa, synthetic construct
  REGION 1..25: note = Description of sequence: 2599_IL1RL1_p.V382F; Type of sequence: Mutation/Peptide
TRNDGKLYDAYVFYPRNYKSSTDGA

SEQ ID NO: 106 - protein, 25 aa, synthetic construct
  REGION 1..25: note = Description of sequence: 2599_AMER3_p.R839C; Type of sequence: Mutation/Peptide
ASAPECRCSLLACEGLLCGQPEVGA

SEQ ID NO: 107 - protein, 25 aa, synthetic construct
  REGION 1..25: note = Description of sequence: 2599_LCT_p.S1404I; Type of sequence: Mutation/Peptide
IEGAWRADGKGLIIWDTFSHTPLRV

SEQ ID NO: 108 - protein, 25 aa, synthetic construct
  REGION 1..25: note = Description of sequence: 2599_DPP4_p.G584V; Type of sequence: Mutation/Peptide
NIIVASFDGRGSVYQGDKIMHAINR

SEQ ID NO: 109 - protein, 25 aa, synthetic construct
  REGION 1..25: note = Description of sequence: 2599_TLK1_p.R370C; Type of sequence: Mutation/Peptide
EDIERQRKLLAKCKPPTANNSQAPS

SEQ ID NO: 110 - protein, 25 aa, synthetic construct
  REGION 1..25: note = Description of sequence: 2599_FSIP2_p.Q1257H; Type of sequence: Mutation/Peptide
QMFSVSEISTVAHEITDSVLNILHK

SEQ ID NO: 111 - protein, 25 aa, synthetic construct
  REGION 1..25: note = Description of sequence: 2599_CFAP44_p.T1734N; Type of sequence: Mutation/Peptide
KQLIREKREMTKNIHKMEETVRQLM

SEQ ID NO: 112 - protein, 25 aa, synthetic construct
  REGION 1..25: note = Description of sequence: 2599_CLSTN2_p.K357N; Type of sequence: Mutation/Peptide
AGLLVDSSEMIFNFDGRQGAKVPDG

SEQ ID NO: 113 - protein, 15 aa, synthetic construct
  REGION 1..15: note = Description of sequence: 2599_FGD5_p.S1503T; Type of sequence: Mutation/Peptide
SAQRWIEAMEDATVL

SEQ ID NO: 114 - protein, 25 aa, synthetic construct
  REGION 1..25: note = Description of sequence: 2599_LRRC2_p.E343V; Type of sequence: Mutation/Peptide
MESERDRQHFDKVVMKAYIEDLKER

SEQ ID NO: 115 - protein, 25 aa, synthetic construct
  REGION 1..25: note = Description of sequence: 2599_PDZRN3_p.Q217R; Type of sequence: Mutation/Peptide
AQLELQMTALRYRKKFTEYSARLDS

SEQ ID NO: 116 - protein, 25 aa, synthetic construct
  REGION 1..25: note = Description of sequence: 2599_CRYBG3_p.S1137F; Type of sequence: Mutation/Peptide
FQEHFGIYTGKIFIDFPTAAQFDNL

SEQ ID NO: 117 - protein, 25 aa, synthetic construct
  REGION 1..25: note = Description of sequence: 2599_FAT4_p.L4520F; Type of sequence: Mutation/Peptide
PAIVGSCATVLAFLVLSLILCNQCR

SEQ ID NO: 118 - protein, 15 aa, synthetic construct
  REGION 1..15: note = Description of sequence: 2599_CCDC149_p.V527M; Type of sequence: Mutation/Peptide
KGIPEGGGMRSTMKT

SEQ ID NO: 119 - protein, 25 aa, synthetic construct
  REGION 1..25: note = Description of sequence: 2599_EVC_p.E200G; Type of sequence: Mutation/Peptide
LSRTFLRVNAFPGVLACESVDVDLC

SEQ ID NO: 120 - protein, 14 aa, synthetic construct
  REGION 1..14: note = Description of sequence: 2599_NPFFR2_p.E521K; Type of sequence: Mutation/Peptide
VMEELKETTNSSKI

SEQ ID NO: 121 - protein, 25 aa, synthetic construct
  REGION 1..25: note = Description of sequence: 2599_KLHL3_p.R352Q; Type of sequence: Mutation/Peptide
DQWTSIASMQERQSTLGAAVLNDLL

SEQ ID NO: 122 - protein, 25 aa, synthetic construct
  REGION 1..25: note = Description of sequence: 2599_PCDHA9_p.E361K; Type of sequence: Mutation/Peptide
QLTIKTLSVPVKKDAQLGTVIALIS

SEQ ID NO: 123 - protein, 25 aa, synthetic construct
  REGION 1..25: note = Description of sequence: 2599_PCDHA9_p.R428Q; Type of sequence: Mutation/Peptide
ESVSAYELVVTAQDRGSPSLWATAR

SEQ ID NO: 124 - protein, 25 aa, synthetic construct
  REGION 1..25: note = Description of sequence: 2599_GRIA1_p.V782I; Type of sequence: Mutation/Peptide
VNLAVLKLSEQGILDKLKSKWWYDK

SEQ ID NO: 125 - protein, 19 aa, synthetic construct
  REGION 1..19: note = Description of sequence: 2599_RXFP3_p.L463M; Type of sequence: Mutation/Peptide
GVVVYSGGRYDLMPSSSAY

SEQ ID NO: 126 - protein, 25 aa, synthetic construct
  REGION 1..25: note = Description of sequence: 2599_GDNF_p.D53H; Type of sequence: Mutation/Peptide
AANMPEDYPDQFHDVMDFIQATIKR

SEQ ID NO: 127 - protein, 25 aa, synthetic construct
  REGION 1..25: note = Description of sequence: 2599_GDNF_p.D79H; Type of sequence: Mutation/Peptide
DSNMPEDYPDQFHDVMDFIQATIKR

SEQ ID NO: 128 - protein, 25 aa, synthetic construct
  REGION 1..25: note = Description of sequence: 2599_STX11_p.R129W; Type of sequence: Mutation/Peptide
QHGPHSAVARISWAQYNALTLTFQR

SEQ ID NO: 129 - protein, 25 aa, synthetic construct
  REGION 1..25: note = Description of sequence: 2599_OR2B3_p.C72Y; Type of sequence: Mutation/Peptide
YFFLTNLSILDLYYTTTTVPHMLVN

SEQ ID NO: 130 - protein, 25 aa, synthetic construct
  REGION 1..25: note = Description of sequence: 2599_THSD7A_p.G1498C; Type of sequence: Mutation/Peptide
GSSRTVWCQRSDCINVTGGCLVMSQ

SEQ ID NO: 131 - protein, 25 aa, synthetic construct
  REGION 1..25: note = Description of sequence: 2599_FLNC_p.V749M; Type of sequence: Mutation/Peptide
KHTIIISWGGVNMPKSPFRVNVGEG

SEQ ID NO: 132 - protein, 25 aa, synthetic construct
  REGION 1..25: note = Description of sequence: 2599_SVOPL_p.A319G; Type of sequence: Mutation/Peptide
ERDLVCGSKSDSGVVVTGGDSGESQ

SEQ ID NO: 133 - protein, 25 aa, synthetic construct
  REGION 1..25: note = Description of sequence: 2599_SLC37A3_p.G396S; Type of sequence: Mutation/Peptide
AKETGSHIEGVTSARETERTMSATS

SEQ ID NO: 134 - protein, 25 aa, synthetic construct
  REGION 1..25: note = Description of sequence: 2599_DNAH11_p.A3906V; Type of sequence: Mutation/Peptide
YALRNFVEEKLGVKYVERTRLDLVK

SEQ ID NO: 135 - protein, 25 aa, synthetic construct
  REGION 1..25: note = Description of sequence: 2599_CRHR2_p.E301K; Type of sequence: Mutation/Peptide
ILMTKLRASTTSKTIQYRKAVKATL

SEQ ID NO: 136 - protein, 25 aa, synthetic construct
  REGION 1..25: note = Description of sequence: 2599_TECPR1_p.G738E; Type of sequence: Mutation/Peptide
PSPQAIWSITCKEDIFVSEPSPDLE

SEQ ID NO: 137 - protein, 25 aa, synthetic construct
  REGION 1..25: note = Description of sequence: 2599_DCAF13_p.L171F; Type of sequence: Mutation/Peptide
SRNPDNYVRETKFDLQRVPRNYDPA

SEQ ID NO: 138 - protein, 25 aa, synthetic construct
  REGION 1..25: note = Description of sequence: 2599_TMEM2_p.D1358H; Type of sequence: Mutation/Peptide
LGVLEQFIPLQLHEYGCPRATTVRR

SEQ ID NO: 139 - protein, 25 aa, synthetic construct
  REGION 1..25: note = Description of sequence: 2599_MAGEC1_p.D573G; Type of sequence: Mutation/Peptide
PHYFPQSPPQGEGSLSPHYFPQSPQ

SEQ ID NO: 140 - protein, 25 aa, synthetic construct
  REGION 1..25: note = Description of sequence: 2599_PHEX_p.Y327F; Type of sequence: Mutation/Peptide
LGYIKKVIDTRLFPHLKDISPSENV

SEQ ID NO: 141 - protein, 25 aa, synthetic construct
  REGION 1..25: note = Description of sequence: 2599_PCDH19_p.V987M; Type of sequence: Mutation/Peptide
NPMPIRSKSPEHMRNIIALSIEATA

SEQ ID NO: 142 - protein, 10 aa, synthetic construct
  REGION 1..10: note = Description of sequence: 2599_ERGIC2_p.L176P*; Type of sequence: Mutation/Peptide
RIHGHPYVNK

SEQ ID NO: 143 - protein, 25 aa, synthetic construct
  REGION 1..25: note = Description of sequence: 8434_ANXA9_p.P272S; Type of sequence: Mutation/Peptide
ALLGLASVIKNTSLYFADKLHQALQ

SEQ ID NO: 144 - protein, 17 aa, synthetic construct
  REGION 1..17: note = Description of sequence: 8434_ANXA9_p.T268I; Type of sequence: Mutation/Peptide
GSARPSFGDQEHIAVLC

SEQ ID NO: 145 - protein, 25 aa, synthetic construct
  REGION 1..25: note = Description of sequence: 8434_DCHS2_p.D822-1; Type of sequence: Mutation/Peptide
LIPGNVSSLFTIESTTGIIYLTLPL

SEQ ID NO: 146 - protein, 25 aa, synthetic construct
  REGION 1..25: note = Description of sequence: 8434_PRRC2C_p.P1763S; Type of sequence: Mutation/Peptide
LASAPLPPSTLASVSASASVSASVP

SEQ ID NO: 147 - protein, 25 aa, synthetic construct
  REGION 1..25: note = Description of sequence: 8434_CRB1_p.Q980H; Type of sequence: Mutation/Peptide
AEKEPEFLNISIHDSRLFFQLQSGN

SEQ ID NO: 148 - protein, 25 aa, synthetic construct
  REGION 1..25: note = Description of sequence: 8434_TRIM67_p.V316M; Type of sequence: Mutation/Peptide
PEHEMENYSMYCMSCRTPVCYLCLE

SEQ ID NO: 149 - protein, 25 aa, synthetic construct
  REGION 1..25: note = Description of sequence: 8434_RYR2_p.H1587R; Type of sequence: Mutation/Peptide
HKNPVPQCPPRLRVQFLSHVLWSRM

SEQ ID NO: 150 - protein, 25 aa, synthetic construct
  REGION 1..25: note = Description of sequence: 8434_SPOCD1_p.R902C; Type of sequence: Mutation/Peptide
SCRLVQALPTVICSAGCIPSNIVWD

SEQ ID NO: 151 - protein, 25 aa, synthetic construct
  REGION 1..25: note = Description of sequence: 8434_ARHGEF16_p.R150W; Type of sequence: Mutation/Peptide
DKDPGGMLRRNLWNQSYRAAMKGLG

SEQ ID NO: 152 - protein, 25 aa, synthetic construct
  REGION 1..25: note = Description of sequence: 8434_CSMD2_p.R655H; Type of sequence: Mutation/Peptide
HCVWLILARPESHIHLAFNDIDVEP

SEQ ID NO: 153 - protein, 25 aa, synthetic construct
  REGION 1..25: note = Description of sequence: 8434_ZCCHC11_p.L355F; Type of sequence: Mutation/Peptide
SLPPPSPAHLAAFSVAVIELAKEHG

SEQ ID NO: 154 - protein, 25 aa, synthetic construct
  REGION 1..25: note = Description of sequence: 8434_SGMS1_p.I228L; Type of sequence: Mutation/Peptide
FCIVGTLYLYRCLTMYVTTLPVPGM

SEQ ID NO: 155 - protein, 25 aa, synthetic construct
  REGION 1..25: note = Description of sequence: 8434_DYNC2H1_p.E869A; Type of sequence: Mutation/Peptide
HKEWIVIGQVDMAALVEKHLFTVHD

SEQ ID NO: 156 - protein, 25 aa, synthetic construct
  REGION 1..25: note = Description of sequence: 8434_CWF19L2_p.N540Y; Type of sequence: Mutation/Peptide
TITQIPKKSGVEYEDQQEVILVRTD

SEQ ID NO: 157 - protein, 25 aa, synthetic construct
  REGION 1..25: note = Description of sequence: 8434_ESAM_p.S110Y; Type of sequence: Mutation/Peptide
VSLVYSMPSRNLYLRLEGLQEKDSG

SEQ ID NO: 158 - protein, 25 aa, synthetic construct
  REGION 1..25: note = Description of sequence: 8434_METTL15_p.P30A; Type of sequence: Mutation/Peptide
WLESGIPNLGVWAKRIHTTAEKYRE

SEQ ID NO: 159 - protein, 25 aa, synthetic construct
  REGION 1..25: note = Description of sequence: 8434_LRP4_p.G1432R; Type of sequence: Mutation/Peptide
ETVIGRGLKTTDRLAVDWVARNLYW

SEQ ID NO: 160 - protein, 13 aa, synthetic construct
  REGION 1..13: note = Description of sequence: 8434_FOLH1_p.L720Q; Type of sequence: Mutation/Peptide
VQAAAETLSEVAQ

SEQ ID NO: 161 - protein, 25 aa, synthetic construct
  REGION 1..25: note = Description of sequence: 8434_UTP20_p.V1905I; Type of sequence: Mutation/Peptide
VRGYQVHVLTFTIHMLLQGLTNKLQ

SEQ ID NO: 162 - protein, 25 aa, synthetic construct
  REGION 1..25: note = Description of sequence: 8434_KRAS_p.Q61H; Type of sequence: Mutation/Peptide
ETCLLDILDTAGHEEYSAMRDQYMR

SEQ ID NO: 163 - protein, 25 aa, synthetic construct
  REGION 1..25: note = Description of sequence: 8434_PARP4_p.H490Q; Type of sequence: Mutation/Peptide
FSDSLSTSIKYSQPGETDGTRLLLI

SEQ ID NO: 164 - protein, 25 aa, synthetic construct
  REGION 1..25: note = Description of sequence: 8434_NPAS3_p.R568L; Type of sequence: Mutation/Peptide
KVERYVESESDLLLQNCESLTSDSA

SEQ ID NO: 165 - protein, 25 aa, synthetic construct
  REGION 1..25: note = Description of sequence: 8434_BAZ1A_p.D514N; Type of sequence: Mutation/Peptide
TKDLTEALDEDANPTKSALSAVASL

SEQ ID NO: 166 - protein, 25 aa, synthetic construct
  REGION 1..25: note = Description of sequence: 8434_FANCM_p.K1138N; Type of sequence: Mutation/Peptide
LNSKSESLPVSDNTAISETPLVSQF

SEQ ID NO: 167 - protein, 25 aa, synthetic construct
  REGION 1..25: note = Description of sequence: 8434_ASB2_p.A528V; Type of sequence: Mutation/Peptide
PPAPQPSSRFNDVPAADKEPSVVQF

SEQ ID NO: 168 - protein, 25 aa, synthetic construct
  REGION 1..25: note = Description of sequence: 8434_RYR3_p.R4693C; Type of sequence: Mutation/Peptide
VYLYTVVAFNFFCKFYNKSEDDDEP

SEQ ID NO: 169 - protein, 25 aa, synthetic construct
  REGION 1..25: note = Description of sequence: 8434_C15orf41_p.R54T; Type of sequence: Mutation/Peptide
SIFSQEYQKHIKTTHAKHHTSEAIE

SEQ ID NO: 170 - protein, 25 aa, synthetic construct
  REGION 1..25: note = Description of sequence: 8434_FEM1B_p.N435T; Type of sequence: Mutation/Peptide
NRVKNISDADVHTAMDNYECNLYTF

SEQ ID NO: 171 - protein, 25 aa, synthetic construct
  REGION 1..25: note = Description of sequence: 8434_PRR35_p.R359Q; Type of sequence: Mutation/Peptide
PKASPSLTRFCSQSSLPTGSSVMLW

SEQ ID NO: 172 - protein, 25 aa, synthetic construct
  REGION 1..25: note = Description of sequence: 8434_BEAN1_p.R14H; Type of sequence: Mutation/Peptide
SFKRPCPLARYNHTSYFYPTFSESS

SEQ ID NO: 173 - protein, 24 aa, synthetic construct
  REGION 1..24: note = Description of sequence: 8434_CES2_p.E612K; Type of sequence: Mutation/Peptide
QFWKKALPQKIQKLEEPEERHTEL

SEQ ID NO: 174 - protein, 25 aa, synthetic construct
  REGION 1..25: note = Description of sequence: 8434_DHX38_p.V724M; Type of sequence: Mutation/Peptide
DILFSKTPQEDYMEAAVKQSLQVHL

SEQ ID NO: 175 - protein, 25 aa, synthetic construct
  REGION 1..25: note = Description of sequence: 8434_STUB1_p.Y164C; Type of sequence: Mutation/Peptide
ERRIHQESELHSCLSRLIAAERERE

SEQ ID NO: 176 - protein, 25 aa, synthetic construct
  REGION 1..25: note = Description of sequence: 8434_GRIN2A_p.V440I; Type of sequence: Mutation/Peptide
TCVRNTVPCRKFIKINNSTNEGMNV

SEQ ID NO: 177 - protein, 25 aa, synthetic construct
  REGION 1..25: note = Description of sequence: 8434_HS3ST3B1_p.P161S; Type of sequence: Mutation/Peptide
GTRALLEFLRVHSDVRAVGAEPHFF

SEQ ID NO: 178 - protein, 25 aa, synthetic construct
  REGION 1..25: note = Description of sequence: 8434_TMEM132E_p.Y916C; Type of sequence: Mutation/Peptide
VFLINCIVFVLRCRHKRIPPEGQTS

SEQ ID NO: 179 - protein, 25 aa, synthetic construct
  REGION 1..25: note = Description of sequence: 8434_TP53_p.R248Q; Type of sequence: Mutation/Peptide
YMCNSSCMGGMNQRPILTIITLEDS

SEQ ID NO: 180 - protein, 25 aa, synthetic construct
  REGION 1..25: note = Description of sequence: 8434_SALL3_p.A826E; Type of sequence: Mutation/Peptide
AATDPAKPLLSYEGSCPPSPPSVIS

SEQ ID NO: 181 - protein, 25 aa, synthetic construct
  REGION 1..25: note = Description of sequence: 8434_ZNF443_p.N185I; Type of sequence: Mutation/Peptide
CKECGKSFSSLGILQRHMAVQRGDG

SEQ ID NO: 182 - protein, 25 aa, synthetic construct
  REGION 1..25: note = Description of sequence: 8434_CACNA1A_p.R2486H; Type of sequence: Mutation/Peptide
NGYYPAHGLARPHGPGSRKGLHEPY

SEQ ID NO: 183 - protein, 25 aa, synthetic construct
  REGION 1..25: note = Description of sequence: 8434_BABAM1_p.A300S; Type of sequence: Mutation/Peptide
ALELHNCMAKLLSHPLQRPCQSHAS

SEQ ID NO: 184 - protein, 25 aa, synthetic construct
  REGION 1..25: note = Description of sequence: 8434_NPHS1_p.S505Y; Type of sequence: Mutation/Peptide
ESRRVHLGSVEKYGSTFSRELVLVT

SEQ ID NO: 185 - protein, 25 aa, synthetic construct
  REGION 1..25: note = Description of sequence: 8434_PNMAL2_p.R432C; Type of sequence: Mutation/Peptide
QPDLPPQAKKAGCGLFGGWSEHRED

SEQ ID NO: 186 - protein, 25 aa, synthetic construct
  REGION 1..25: note = Description of sequence: 8434_SBK2_p.A115T; Type of sequence: Mutation/Peptide
FLYEFCVGLSLGTHSAIVTAYGIGI

SEQ ID NO: 187 - protein, 25 aa, synthetic construct
  REGION 1..25: note = Description of sequence: 8434_ZNF865_p.V397A; Type of sequence: Mutation/Peptide
KSFNRRESLKRHAKTHSADLLRLPC

SEQ ID NO: 188 - protein, 25 aa, synthetic construct
  REGION 1..25: note = Description of sequence: 8434_REV1_p.E1069G; Type of sequence: Mutation/Peptide
KNPLLHLKAAVKGKKRNKKKKTIGS

SEQ ID NO: 189 - protein, 25 aa, synthetic construct
  REGION 1..25: note = Description of sequence: 8434_OLA1_p.I225N; Type of sequence: Mutation/Peptide
QGRNYIVEDGDINFFKFNTPQQPKK

SEQ ID NO: 190 - protein, 25 aa, synthetic construct
  REGION 1..25: note = Description of sequence: 8434_ABCA12_p.A2248S; Type of sequence: Mutation/Peptide
SQTTLEEVFINFSKDQKSYETADTS

SEQ ID NO: 191 - protein, 25 aa, synthetic construct
  REGION 1..25: note = Description of sequence: 8434_EPAS1_p.D810N; Type of sequence: Mutation/Peptide
RFPPQCYATQYQNYSLSSAHKVSGM

SEQ ID NO: 192 - protein, 25 aa, synthetic construct
  REGION 1..25: note = Description of sequence: 8434_DOPEY2_p.N2059I; Type of sequence: Mutation/Peptide
LYLPLIQERLTDILRVGQTSIVAAQ

SEQ ID NO: 193 - protein, 17 aa, synthetic construct
  REGION 1..17: note = Description of sequence: 8434_TXNRD2_p.N334K; Type of sequence: Mutation/Peptide
ACLPTTVGHAGKKQRRD

SEQ ID NO: 194 - protein, 25 aa, synthetic construct
  REGION 1..25: note = Description of sequence: 8434_CELSR1_p.R1288W; Type of sequence: Mutation/Peptide
PSEDLQEQIYLNWTLLTTISTQRVL

SEQ ID NO: 195 - protein, 25 aa, synthetic construct
  REGION 1..25: note = Description of sequence: 8434_MYLK_p.S1175T; Type of sequence: Mutation/Peptide
CASDIRSSSLTLTWYGSSYDGGSAV

SEQ ID NO: 196 - protein, 25 aa, synthetic construct
  REGION 1..25: note = Description of sequence: 8434_KCNAB1_p.G25S; Type of sequence: Mutation/Peptide
ISEENTKLRRQSSFSVAGKDKSPKK

SEQ ID NO: 197 - protein, 19 aa, synthetic construct
  REGION 1..19: note = Description of sequence: 8434_SI_p.S7C; Type of sequence: Mutation/Peptide
MARKKFCGLEISLIVLFVI

SEQ ID NO: 198 - protein, 25 aa, synthetic construct
  REGION 1..25: note = Description of sequence: 8434_RAB5A_p.G27E; Type of sequence: Mutation/Peptide
GNKICQFKLVLLEESAVGKSSLVLR

SEQ ID NO: 199 - protein, 25 aa, synthetic construct
  REGION 1..25: note = Description of sequence: 8434_CSPG5_p.T440M; Type of sequence: Mutation/Peptide
SAALVLLLLFMMMVFFAKKLYLLKT

SEQ ID NO: 200 - protein, 25 aa, synthetic construct
  REGION 1..25: note = Description of sequence: 8434_BSN_p.M1191L; Type of sequence: Mutation/Peptide
RPLKSAEEAYEELMRKAELLQRQQG

SEQ ID NO: 201 - protein, 25 aa, synthetic construct
  REGION 1..25: note = Description of sequence: 8434_ITIH3_p.T862M; Type of sequence: Mutation/Peptide
SQKDYRKDASIGMKVVCWFVHNNGE

SEQ ID NO: 202 - protein, 25 aa, synthetic construct
  REGION 1..25: note = Description of sequence: 8434_OR5H14_p.R143Q; Type of sequence: Mutation/Peptide
LYPAIMTNGLCIQLLILSYVGGLLH

SEQ ID NO: 203 - protein, 25 aa, synthetic construct
  REGION 1..25: note = Description of sequence: 8434_TRPC3_p.N829I; Type of sequence: Mutation/Peptide
NSKSRLNLFTQSISRVFESHSFNSI

SEQ ID NO: 204 - protein, 25 aa, synthetic construct
  REGION 1..25: note = Description of sequence: 8434_FBXW7_p.H580Q; Type of sequence: Mutation/Peptide
VETGNCIHTLTGQQSLTSGMELKDN

SEQ ID NO: 205 - protein, 25 aa, synthetic construct
  REGION 1..25: note = Description of sequence: 8434_NSD2_p.R1353W; Type of sequence: Mutation/Peptide
PPPEPGKPKGKRWRRRGWRRVTEGK

SEQ ID NO: 206 - protein, 25 aa, synthetic construct
  REGION 1..25: note = Description of sequence: 8434_MYO10_p.R1166H; Type of sequence: Mutation/Peptide
SRFDTDDELSYRHDSVYSCVTLPYF

SEQ ID NO: 207 - protein, 25 aa, synthetic construct
  REGION 1..25: note = Description of sequence: 8434_ROS1_p.R2083S; Type of sequence: Mutation/Peptide
ERMHFIHRDLAASNCLVSVKDYTSP

SEQ ID NO: 208 - protein, 25 aa, synthetic construct
  REGION 1..25: note = Description of sequence: 8434_MCM9_p.V1041A; Type of sequence: Mutation/Peptide
AHLTCEGDKKEEASGSNKSGKVHAC

SEQ ID NO: 209 - protein, 25 aa, synthetic construct
  REGION 1..25: note = Description of sequence: 8434_FNDC1_p.A19V; Type of sequence: Mutation/Peptide
ATLRAPRRLSWAVLLLLAALLPVAS

SEQ ID NO: 210 - protein, 25 aa, synthetic construct
  REGION 1..25: note = Description of sequence: 8434_RGL2_p.G551E; Type of sequence: Mutation/Peptide
GSPLSGGAEEASEGTGYGGEGSGPG

SEQ ID NO: 211 - protein, 25 aa, synthetic construct
  REGION 1..25: note = Description of sequence: 8434_TOP1MT_p.K231E; Type of sequence: Mutation/Peptide
FIDKLALRAGNEEEDGEAADTVGCC

SEQ ID NO: 212 - protein, 25 aa, synthetic construct
  REGION 1..25: note = Description of sequence: 8434_TMEM55A_p.N89T; Type of sequence: Mutation/Peptide
KCTVCNEATPIKTPPTGKKYVRCPC

SEQ ID NO: 213 - protein, 25 aa, synthetic construct
  REGION 1..25: note = Description of sequence: 8434_COL4A5_p.G270E; Type of sequence: Mutation/Peptide
GDQGLPGDRGPPEPPGIRGPPGPPG

SEQ ID NO: 214 - protein, 23 aa, synthetic construct
  REGION 1..23: note = Description of sequence: 8434_ATP11C_p.L1109F; Type of sequence: Mutation/Peptide
SARNPNLELPMLFSYKHTDSGYS

SEQ ID NO: 215 - protein, 25 aa, synthetic construct
  REGION 1..25: note = Description of sequence: 8434_DCHS2_p.D228E-2; Type of sequence: Mutation/Peptide
LIPGNVSSLFTIESTTGLYSPEVEI

SEQ ID NO: 216 - protein, 25 aa, synthetic construct
  REGION 1..25: note = Description of sequence: 8434_PRRC2C_p.P1808S; Type of sequence: Mutation/Peptide
STSAPVPASPLASVSASASVSASVP

SEQ ID NO: 217 - protein, 25 aa, synthetic construct
  REGION 1..25: note = Description of sequence: 6932_KDELC2_p.D213G; Type of sequence: Mutation/Peptide
NHVYRRSLGKYTGFKMFSDEILLSL

SEQ ID NO: 218 - protein, 25 aa, synthetic construct
  REGION 1..25: note = Description of sequence: 6932_SLC5A12_p.G581E; Type of sequence: Mutation/Peptide
QENLENGSARKQEAESVLQNGLRRE

SEQ ID NO: 219 - protein, 25 aa, synthetic construct
  REGION 1..25: note = Description of sequence: 6932_CMKLR1_p.P198S; Type of sequence: Mutation/Peptide
KISCFNNFSLSTSGSSSWPTHSQMD

SEQ ID NO: 220 - protein, 25 aa, synthetic construct
  REGION 1..25: note = Description of sequence: 6932_RIMBP2_p.R173Q; Type of sequence: Mutation/Peptide
ARCRSESDMENEQNSNTSKQRYSGK

SEQ ID NO: 221 - protein, 25 aa, synthetic construct
  REGION 1..25: note = Description of sequence: 6932_AKAP3_p.V551I; Type of sequence: Mutation/Peptide
LAQGGRRDARSFIEAAGTTNFPANE

SEQ ID NO: 222 - protein, 25 aa, synthetic construct
  REGION 1..25: note = Description of sequence: 6932_ATP11A_p.A955T; Type of sequence: Mutation/Peptide
VLKRDPTLYRDVTKNALLRWRVFIY

SEQ ID NO: 223 - protein, 25 aa, synthetic construct
  REGION 1..25: note = Description of sequence: 6932_NDFIP2_p.G72A; Type of sequence: Mutation/Peptide
GGRGPAATTSSTAVAVGAEHGEDSL

SEQ ID NO: 224 - protein, 25 aa, synthetic construct
  REGION 1..25: note = Description of sequence: 6932_CSK_p.D115Y; Type of sequence: Mutation/Peptide
LFLVRESTNYPGYYTLCVSCDGKVE

SEQ ID NO: 225 - protein, 25 aa, synthetic construct
  REGION 1..25: note = Description of sequence: 6932_IGF1R_p.R222Q; Type of sequence: Mutation/Peptide
RCQKMCPSTCGKQACTENNECCHPE

SEQ ID NO: 226 - protein, 25 aa, synthetic construct
  REGION 1..25: note = Description of sequence: 6932_RHBDL1_p.R154W; Type of sequence: Mutation/Peptide
KRAIANGQRALPWDGPLDEPGLGVY

SEQ ID NO: 227 - protein, 25 aa, synthetic construct
  REGION 1..25: note = Description of sequence: 6932_HGS_p.F145I; Type of sequence: Mutation/Peptide
QIMKVEGHVFPEIKESDAMFAAERA

SEQ ID NO: 228 - protein, 25 aa, synthetic construct
  REGION 1..25: note = Description of sequence: 6932_MAPK4_p.V541E; Type of sequence: Mutation/Peptide
VDGGASPQFDLDEFISRALKLCTKP

SEQ ID NO: 229 - protein, 25 aa, synthetic construct
  REGION 1..25: note = Description of sequence: 6932_SERPINB4_p.E353K; Type of sequence: Mutation/Peptide
VEAAAATAVVVVKLSSPSTNEEFCC

SEQ ID NO: 230 - protein, 25 aa, synthetic construct
  REGION 1..25: note = Description of sequence: 6932_BTBD2_p.T157K; Type of sequence: Mutation/Peptide
VFDAMFNGGMATKSTEIELPDVEPA

SEQ ID NO: 231 - protein, 25 aa, synthetic construct
  REGION 1..25: note = Description of sequence: 6932_ZNF114_p.T241A; Type of sequence: Mutation/Peptide
AFREDGSLRAHNAHGREKMYDFTQC

SEQ ID NO: 232 - protein, 25 aa, synthetic construct
  REGION 1..25: note = Description of sequence: 6932_MCOLN1_p.D546H; Type of sequence: Mutation/Peptide
ESELQAYIAQCQHSPTSGKFRRGSG

SEQ ID NO: 233 - protein, 25 aa, synthetic construct
  REGION 1..25: note = Description of sequence: 6932_RYR2_p.R2303S; Type of sequence: Mutation/Peptide
NPVEGERYLDFLSFAVFCNGESVEE

SEQ ID NO: 234 - protein, 25 aa, synthetic construct
  REGION 1..25: note = Description of sequence: 6932_OR2T10_p.A163S; Type of sequence: Mutation/Peptide
VGSVDGFMLTPISMSFPFCRSHEIQ

SEQ ID NO: 235 - protein, 25 aa, synthetic construct
  REGION 1..25: note = Description of sequence: 6932_CC2D1B_p.R550H; Type of sequence: Mutation/Peptide
KLQYQRAALQAKHSQDLEQAKAYLR

SEQ ID NO: 236 - protein, 25 aa, synthetic construct
  REGION 1..25: note = Description of sequence: 6932_ZZZ3_p.R46L; Type of sequence: Mutation/Peptide
AHPEEISSNSQVLSRSPKKRPEPVP

SEQ ID NO: 237 - protein, 25 aa, synthetic construct
  REGION 1..25: note = Description of sequence: 6932_THBD_p.R540C; Type of sequence: Mutation/Peptide
LVVALLALLCHLCKKQGAARAKMEY

SEQ ID NO: 238 - protein, 25 aa, synthetic construct
  REGION 1..25: note = Description of sequence: 6932_HELZ2_p.P775A; Type of sequence: Mutation/Peptide
NPIHARGKVPPHARHYPLMFCHVAG

SEQ ID NO: 239 - protein, 25 aa, synthetic construct
  REGION 1..25: note = Description of sequence: 6932_EVX2_p.Y144F; Type of sequence: Mutation/Peptide
GAAQLKENNGKGFAESGSAAGTTTS

SEQ ID NO: 240 - protein, 25 aa, synthetic construct
  REGION 1..25: note = Description of sequence: 6932_D2HGDH_p.V173I; Type of sequence: Mutation/Peptide
SVSGILVCQAGCILEELSRYVEERD

SEQ ID NO: 241 - protein, 25 aa, synthetic construct
  REGION 1..25: note = Description of sequence: 6932_CD207_p.R43C; Type of sequence: Mutation/Peptide
GPSLVPGKTPTVCAALICLTLVLVA

SEQ ID NO: 242 - protein, 25 aa, synthetic construct
  REGION 1..25: note = Description of sequence: 6932_PIK3CA_p.H1047R; Type of sequence: Mutation/Peptide
ALEYFMKQMNDARHGGWTTKMDWIF

SEQ ID NO: 243 - protein, 24 aa, synthetic construct
  REGION 1..24: note = Description of sequence: 6932_MTMR14_p.G639V; Type of sequence: Mutation/Peptide
GAIGGLLEQFARVVGLRSISSNAL

SEQ ID NO: 244 - protein, 25 aa, synthetic construct
  REGION 1..25: note = Description of sequence: 6932_GABRB1_p.P119Q; Type of sequence: Mutation/Peptide
TLDNRVADQLWVQDTYFLNDKKSFV

SEQ ID NO: 245 - protein, 25 aa, synthetic construct
  REGION 1..25: note = Description of sequence: 6932_PCDHAC2_p.G315W; Type of sequence: Mutation/Peptide
RERQLFSIDASTWEVRVIGGLDYEE

SEQ ID NO: 246 - protein, 25 aa, synthetic construct
  REGION 1..25: note = Description of sequence: 6932_ZFPM2_p.N170K; Type of sequence: Mutation/Peptide
LDVTWQGVEDNKKNCIVYSKEDIFP

SEQ ID NO: 247 - protein, 25 aa, synthetic construct
  REGION 1..25: note = Description of sequence: 6932_ZFPM2_p.N170K*; Type of sequence: Mutation/Peptide
LDVTWQGVEDNKKNCIVYSKGGQLW

SEQ ID NO: 248 - protein, 25 aa, synthetic construct
  REGION 1..25: note = Description of sequence: 6932_BCORL1_p.P823L; Type of sequence: Mutation/Peptide
ANIYPRCSVNGKLTSTQVLPVGWSP

SEQ ID NO: 249 - protein, 17 aa, synthetic construct
  REGION 1..17: note = Description of sequence: 6932_ZNRF3_p.D556fs; Type of sequence: Mutation/Peptide
GEPWPGPASPSGDAAWR

SEQ ID NO: 250 - protein, 25 aa, synthetic construct
  REGION 1..25: note = Description of sequence: 0025_ANO5_p.V634I; Type of sequence: Mutation/Peptide
SGATVTLWMSLVITSMVAVIVYRLS

SEQ ID NO: 251 - protein, 25 aa, synthetic construct
  REGION 1..25: note = Description of sequence: 0025_CHST1_p.R125W; Type of sequence: Mutation/Peptide
RRVMLGASRDLLWSLYDCDLYFLEN

SEQ ID NO: 252 - protein, 25 aa, synthetic construct
  REGION 1..25: note = Description of sequence: 0025_CCDC88B_p.R1300H; Type of sequence: Mutation/Peptide
KQKLVEKIMDQYHVLEPVPLPRTKK

SEQ ID NO: 253 - protein, 25 aa, synthetic construct
  REGION 1..25: note = Description of sequence: 0025_TENM4_p.R2592Q; Type of sequence: Mutation/Peptide
TTDIISVANEDGQRVAAILNHAHYL

SEQ ID NO: 254 - protein, 24 aa, synthetic construct
  REGION 1..24: note = Description of sequence: 0025_KRAS_p.G12C; Type of sequence: Mutation/Peptide
MTEYKLVVVGACGVGKSALTIQLI

SEQ ID NO: 255 - protein, 25 aa, synthetic construct
  REGION 1..25: note = Description of sequence: 0025_SCAF11_p.T945S; Type of sequence: Mutation/Peptide
RRQSQSRSPKRDSTRESRRSESLSP

SEQ ID NO: 256 - protein, 25 aa, synthetic construct
  REGION 1..25: note = Description of sequence: 0025_AMDHD1_p.R127C; Type of sequence: Mutation/Peptide
AGGGIHFTVERTCQATEEELFRSLQ

SEQ ID NO: 257 - protein, 25 aa, synthetic construct
  REGION 1..25: note = Description of sequence: 0025_SLC25A29_p.R94C; Type of sequence: Mutation/Peptide
RGVNRGMVSTLLCETPSFGVYFLTY

SEQ ID NO: 258 - protein, 25 aa, synthetic construct
  REGION 1..25: note = Description of sequence: 0025_ZFYVE19_p.R60Q; Type of sequence: Mutation/Peptide
SLELDYHTSSCFQGTMVKADCPVPI

SEQ ID NO: 259 - protein, 20 aa, synthetic construct
  REGION 1..20: note = Description of sequence: 0025_FGF7_p.H187Y; Type of sequence: Mutation/Peptide
RGKKTKKEQKTAYFLPMAIT

SEQ ID NO: 260 - protein, 25 aa, synthetic construct
  REGION 1..25: note = Description of sequence: 0025_MFGE8_p.A277D; Type of sequence: Mutation/Peptide
SYARLDKQGNFNDWVAGSYGNDQWL

SEQ ID NO: 261 - protein, 25 aa, synthetic construct
  REGION 1..25: note = Description of sequence: 0025_ZNF276_p.A372V; Type of sequence: Mutation/Peptide
SMVHPLTQTQDKVLPLEAEPPPGPP

SEQ ID NO: 262 - protein, 25 aa, synthetic construct
  REGION 1..25: note = Description of sequence: 0025_FBF1_p.S674L; Type of sequence: Mutation/Peptide
RRENEELSARYLLQCQEAEQARAEL

SEQ ID NO: 263 - protein, 25 aa, synthetic construct
  REGION 1..25: note = Description of sequence: 0025_RNF157_p.V240I; Type of sequence: Mutation/Peptide
GTFCVKPLKQKQIVDGVSYLLQEIY

SEQ ID NO: 264 - protein, 25 aa, synthetic construct
  REGION 1..25: note = Description of sequence: 0025_RNF213_p.N2935D; Type of sequence: Mutation/Peptide
APHKKVGFVGISDWALDPAKMNRGI

SEQ ID NO: 265 - protein, 25 aa, synthetic construct
  REGION 1..25: note = Description of sequence: 0025_DDX39A_p.I79F; Type of sequence: Mutation/Peptide
PSEVQHECIPQAFLGMDVLCQAKSG

SEQ ID NO: 266 - protein, 25 aa, synthetic construct
  REGION 1..25: note = Description of sequence: 0025_RHPN2_p.S201C; Type of sequence: Mutation/Peptide
TRQMGLLFTWYDCLTGVPVSQQNLL

SEQ ID NO: 267 - protein, 25 aa, synthetic construct
  REGION 1..25: note = Description of sequence: 0025_NLRP9_p.L446Q; Type of sequence: Mutation/Peptide
LQRRGDCFAFMHQCIQEFCAAMFYL

SEQ ID NO: 268 - protein, 25 aa, synthetic construct
  REGION 1..25: note = Description of sequence: 0025_MTOR_p.L2220F; Type of sequence: Mutation/Peptide
LLANDPTSLRKNFSIQRYAVIPLST

SEQ ID NO: 269 - protein, 25 aa, synthetic construct
  REGION 1..25: note = Description of sequence: 0025_LAX1_p.A158S; Type of sequence: Mutation/Peptide
HATEYAVGIYDNSMVPQMCGNLTPS

SEQ ID NO: 270 - protein, 25 aa, synthetic construct
  REGION 1..25: note = Description of sequence: 0025_SOX13_p.A63T; Type of sequence: Mutation/Peptide
PARASQDSADPQTPAQGNFRGSWDC

SEQ ID NO: 271 - protein, 25 aa, synthetic construct
  REGION 1..25: note = Description of sequence: 0025_MN1_p.A831G; Type of sequence: Mutation/Peptide
LFGQSCLAALSTGCQNMIASLGAPN

SEQ ID NO: 272 - protein, 25 aa, synthetic construct
  REGION 1..25: note = Description of sequence: 0025_RPS19BP1_p.D21Y; Type of sequence: Mutation/Peptide
GLELLAASEAPRYPPGQAKPRGAPV

SEQ ID NO: 273 - protein, 25 aa, synthetic construct
  REGION 1..25: note = Description of sequence: 0025_KIAA0930_p.R208C; Type of sequence: Mutation/Peptide
NTFQGVIFQGSICYEALKKVYDNRV

SEQ ID NO: 274 - protein, 20 aa, synthetic construct
  REGION 1..20: note = Description of sequence: 0025_CMBL_p.I238T; Type of sequence: Mutation/Peptide
DKPYIDEARRNLTEWLNKYM

SEQ ID NO: 275 - protein, 25 aa, synthetic construct
  REGION 1..25: note = Description of sequence: 0025_GCNT2_p.N241K; Type of sequence: Mutation/Peptide
TKYVHQELLNHKKSYVIKTTKLKTP

SEQ ID NO: 276 - protein, 25 aa, synthetic construct
  REGION 1..25: note = Description of sequence: 0025_ECI2_p.A37G; Type of sequence: Mutation/Peptide
KDPGNEVKLKLYGLYKQATEGPCNM

SEQ ID NO: 277 - protein, 25 aa, synthetic construct
  REGION 1..25: note = Description of sequence: 0025_GRM8_p.R672H; Type of sequence: Mutation/Peptide
CFSYAALLTKTNHIHRIFEQGKKSV

SEQ ID NO: 278 - protein, 25 aa, synthetic construct
  REGION 1..25: note = Description of sequence: 0025_KLRG2_p.P70L; Type of sequence: Mutation/Peptide
AGAGLEPSSKKKLPSPRPGSPRVPP

SEQ ID NO: 279 - protein, 25 aa, synthetic construct
  REGION 1..25: note = Description of sequence: 0025_RPL8_p.E80K; Type of sequence: Mutation/Peptide
RFKKRTELFIAAKGIHTGQFVYCGK

SEQ ID NO: 280 - protein, 25 aa, synthetic construct
  REGION 1..25: note = Description of sequence: 0025_GFRA2_p.R246H; Type of sequence: Mutation/Peptide
LFCSCQDQACAEHRRQTILPSCSYE

SEQ ID NO: 281 - protein, 25 aa, synthetic construct
  REGION 1..25: note = Description of sequence: 0025_PTCH1_p.D803Y; Type of sequence: Mutation/Peptide
RPHRPEWVHDKAYYMPETRLRIPAA

SEQ ID NO: 282 - protein, 25 aa, synthetic construct
  REGION 1..25: note = Description of sequence: 0025_NYX_p.R268H; Type of sequence: Mutation/Peptide
LRTLNLGGNALDHVARAWFADLAEL

SEQ ID NO: 283 - protein, 25 aa, synthetic construct
  REGION 1..25: note = Description of sequence: 0025_ARID1A_p.D1825fs-1; Type of sequence: Mutation/Peptide
VKIVQKNDPFVVEISLGVCRSLTVA

SEQ ID NO: 284 - protein, 25 aa, synthetic construct
  REGION 1..25: note = Description of sequence: 0025_ARID1A_p.D1825fs-2; Type of sequence: Mutation/Peptide
KNDPFVVEISLGVCRSLTVACCTGG

SEQ ID NO: 285 - protein, 25 aa, synthetic construct
  REGION 1..25: note = Description of sequence: 0025_ARID1A_p.D1825fs-3; Type of sequence: Mutation/Peptide
VVEISLGVCRSLTVACCTGGLVGGT

SEQ ID NO: 286 - protein, 25 aa, synthetic construct
  REGION 1..25: note = Description of sequence: 0025_ARID1A_p.D1825fs-4; Type of sequence: Mutation/Peptide
LGVCRSLTVACCTGGLVGGTPLSIS

SEQ ID NO: 287 - protein, 25 aa, synthetic construct
  REGION 1..25: note = Description of sequence: 0025_ARID1A_p.D1825fs-5; Type of sequence: Mutation/Peptide
SLTVACCTGGLVGGTPLSISRPTSR

SEQ ID NO: 288 - protein, 25 aa, synthetic construct
  REGION 1..25: note = Description of sequence: 0025_ARID1A_p.D1825fs-6; Type of sequence: Mutation/Peptide
CCTGGLVGGTPLSISRPTSRARQSC

SEQ ID NO: 289 - protein, 25 aa, synthetic construct
  REGION 1..25: note = Description of sequence: 0025_ARID1A_p.D1825fs-7; Type of sequence: Mutation/Peptide
LVGGTPLSISRPTSRARQSCCLPGL

SEQ ID NO: 290 - protein, 25 aa, synthetic construct
  REGION 1..25: note = Description of sequence: 0025_ARID1A_p.D1825fs-8; Type of sequence: Mutation/Peptide
PLSISRPTSRARQSCCLPGLTHPAH

SEQ ID NO: 291 - protein, 25 aa, synthetic construct
  REGION 1..25: note = Description of sequence: 0025_ARID1A_p.D1825fs-9; Type of sequence: Mutation/Peptide
ISRPTSRARQSCCLPGLTHPAHQPL

SEQ ID NO: 292 - protein, 25 aa, synthetic construct
  REGION 1..25: note = Description of sequence: 0025_ARID1A_p.D1825fs-10; Type of sequence: Mutation/Peptide
PTSRARQSCCLPGLTHPAHQPLGSM

SEQ ID NO: 293 - protein, 25 aa, synthetic construct
  REGION 1..25: note = Description of sequence: EGFR L858 WT 25mer
KTPQHVKITDFGLAKLLGAEEKEYH

SEQ ID NO: 294 - protein, 25 aa, synthetic construct
  REGION 1..25: note = Description of sequence: EGFR L858R MUT 25mer
KTPQHVKITDFGRAKLLGAEEKEYH

SEQ ID NO: 295 - protein, 9 aa, synthetic construct
  REGION 1..9: note = Description of sequence: EGFR L858 WT 9mer
KITDFGLAK

SEQ ID NO: 296 - protein, 9 aa, synthetic construct
  REGION 1..9: note = Description of sequence: EGFR L858R MUT 9mer
KITDFGRAK

SEQ ID NO: 297 - protein, 10 aa, synthetic construct
  REGION 1..10: note = Description of sequence: EGFR L858 WT 10mer A
VKITDFGLAK

SEQ ID NO: 298 - protein, 10 aa, synthetic construct
  REGION 1..10: note = Description of sequence: EGFR L858R MUT 10mer A
VKITDFGRAK

SEQ ID NO: 299 - protein, 10 aa, synthetic construct
  REGION 1..10: note = Description of sequence: EGFR L858 WT 10mer B
KITDFGLAKL

SEQ ID NO: 300 - protein, 10 aa, synthetic construct
  REGION 1..10: note = Description of sequence: EGFR L858R MUT 10mer B
KITDFGRAKL